SEQUENCE LISTING (19 sequences)

SEQ ID NO: 1 - protein, 1232 aa, mouse
MQLTYPGSLPVIGEKEVVQADDEPTFSFFSGPYMVMTNLVWNRSRVTVKELNLPTRPHCSRLRLADLLIAEQEHSSNLRHPNLLQLMAVCLSRDLEKIRLVYERIAVGTLFSVLHERRSQFPVLHMEVIVHLLLQVADALIYLHSRGFIHRSLSSYAVHIVSAGEARLTNLEYLTESQDSGAHRNVTRMPLPTQLYNWAAPEVVLQKAATVKSDIYSFSVIIQEILTDSIPWNGLDGSLVKETIALGNYLEADVRLPEPYYDIVKSGIHAKQKNRTMNLQDIRYILKNDLKEFIGAQKTQPTESPRGQSYEPHPDVNICLGLTSEYQKDPPDLDIKELKEMGSQPHSPTDHSFLTVKPTLAPQTLDSSLSAQKPDNANVPSPPAACLAEEVRSPTASQDSLCSFEINEIYSGCLTLGTDKEEECLGTAASPEGDRPNQGDELPSLEEELDKMERELHCFCEEDKSISEVDTDLLFEDDDWQSDSLGSLNLPEPTREAKGKTSSWSKTDEYVSKCVLNLKISQVMMQQSAEWLRKLEQEVEELEWAQKELDSQCSSLRDASLKFANAKFQPAVGPPSLAYLPPVMQLPGLKQPENGGTWLTLARSPGNEREFQEGHFSKKPEKLSACGWKPFTQVSEESRGDCSELNNQLPTLRGPGKQSTGEQLPSTQEARESLEKNTNQNSRSMASVSSEIYATKSRNNEDNGEAHLKWRLAVKEMAEKAVSGQLLLPPWNPQSSAPFESKVENESTPLPRPPIRGPESTEWQHILEYQRENDEPKGNTKFGKMDNSDCDKNKHSRWTGLQRFTGIRYPFFRNHEQPEQNEASQASCDTSVGTEKFYSTSSPIGDDFERFQDSFAQRQGYVEENFQIREIFEKNAEILTKPQFQAIQCAEDKQDETLGETPKELKEKNTSLTDIQDLSSITYDQDGYFKETSYKTPKLKHAPTSASTPLSPESISSAASHYEDCLENTTFHVKRGSTFCWNGQEAMRTLSAKFTTVRERAKSLESLLASSKSLPAKLTDSKRLCMLSETGSSNVSAAFVTSTHATKRKSLPRELAEATSQQHLDELPPPAQELLDEIEQLKQQQVSSLASHENTARDLSVTNKDKKHLEEQETNSSKDSSFLSSREIQDLEDTERAHSSLDEDLERFLQSPEENTALLDPTKGSTREKKNKDQDVVEQKRKKKESIKPERRESDSSLGTLEEDELKPCFWKRLGWSEPSRIIVLDQSDLSD

SEQ ID NO: 2 - protein, 1491 aa, human
MSRAVRLPVPCPVQLGTLRNDSLEAQLHEYVKQGNYVKVKKILKKGIYVDAVNSLGQTALFVAALLGLRKFVDVLVDYGSDPNHRCFDGSTPVHAAAFSGNQWILSKLLDAGGDLRLHDERGQNPKTWALTAGKERSTQIVEFMQRCASHMQAIIQGFSYDLLKKIDSPQRLVYSPSWCGGLVQGNPNGSPNRLLKAGVISAQNIYSFGFGKFYLTGATQMAYLGSLPVIGEKEVIQADDEPTFSFFSGPYMVMTNLVWNGSRVTVKELNLPTHPHCSRLRLADLLIAEQEHSSKLRHPYLLQLMAVCLSQDLEKTRLVYERITIGTLFSVLHERRSQFPVLHMEVIVHLLLQISDALRYLHFQGFIHRSLSSYAVHIISPGEARLTNLEYMLESEDRGVQRDLTRVPLPTQLYNWAAPEVILQKAATVKSDIYSFSMIMQEILTDDIPWKGLDGSVVKKAVVSGNYLEADVRLPKPYYDIVKSGIHVKQKDRTMNLQDIRYILKNDLKDFTGAQRTQPTESPRVQRYGLHPDVNVYLGLTSEHPRETPDMEIIELKEMGSQPHSPRVHSLFTEGTLDPQAPDPCLMARETQNQDAPCPAPFMAEEASSPSTGQPSLCSFEINEIYSGCLILEDDIEEPPGAASSLEADGPNQVDELKSMEEELDKMEREACCFGSEDESSSKAETEYSFDDWDWQNGSLSSLSLPESTREAKSNLNNMSTTEEYLISKCVLDLKIMQTIMHENDDRLRNIEQILDEVEMKQKEQEERMSLWATSREFTNAYKLPLAVGPPSLNYIPPVLQLSGGQKPDTSGNYPTLPRFPRMLPTLCDPGKQNTDEQFQCTQGAKDSLETSRIQNTSSQGRPRESTAQAKATQFNSALFTLSSHRQGPSASPSCHWDSTRMSVEPVSSEIYNAESRNKDDGKVHLKWKMEVKEMAKKAATGQLTVPPWHPQSSLTLESEAENEPDALLQPPIRSPENTDWQRVIEYHRENDEPRGNGKFDKTGNNDCDSDQHGRQPRLGSFTSIRHPSPRQKEQPEHSEAFQASSDTLVAVEKSYSHQSMQSTCSPESSEDITDEFLTPDGEYFYSSTAQENLALETSSPIEEDFEGIQGAFAQPQVSGEEKFQMRKILGKNAEILPRSQFQPVRSTEDEQEETSKESPKELKEKDISLTDIQDLSSISYEPDSSFKEASCKTPKINHAPTSVSTPLSPGSVSSAASQYKDCLESITFQVKTEFASCWNSQEFIQTLSDDFISVRERAKKLDSLLTSSETPPSRLTGLKRLSSFIGAGSPSLVKACDSSPPHATQRRSLPKVEAFSQHHIDELPPPSQELLDDIELLKQQQGSSTVLHENTASDGGGTANDQRHLEEQETDSKKEDSSMLLSKETEDLGEDTERAHSTLDEDLERWLQPPEESVELQDLPKGSERETNIKDQKVGEEKRKREDSITPERRKSEGVLGTSEEDELKSCFWKRLGWSESSRIIVLDQSDLSD

SEQ ID NO: 3 - protein, 1451 aa, human
MSRAVRLPVPCPVQLGTLRNDSLEAQLHEYVKQGNYVKVKKILKKGIYVDAVNSLGQTALFVAALLGLRKFVDVLVDYGSDPNHRCFDGSTPVHAAAFSGNQWILSKLLDAGGDLRLHDERGQNPKTWALTAGKERSTQIVEFMQRCASHMQAIIQGFSYDLLKKIDSPQRLVYSPSWCGGLVQGNPNGSPNRLLKAGVISAQNIYSFGFGKFYLTGATQMAYLGSLPVIGEKEVIQADDEPTFSFFSGPYMVMTNLVWNGSRVTVKELNLPTHPHCSRLRLADLLIAEQEHSSKLRHPYLLQLMAVCLSQDLEKTRLVYERITIGTLFSVLHERRSQFPVLHMEVIVHLLLQISDALRYLHFQGFIHRSLSSYAVHIISPGEARLTNLEYMLESEDRGVQRDLTRVPLPTQLYNWAAPEVILQKAATVKSDIYSFSMIMQEILTDDIPWKGLDGSVVKKAVVSGNYLEADVRLPKPYYDIVKSGIHVKQKDRTMNLQDIRYILKNDLKDFTGAQRTQPTESPRVQRYGLHPDVNVYLGLTSEHPRETPDMEIIELKEMGSQPHSPRVHSLFTEGTLDPQAPDPCLMARETQNQDAPCPAPFMAEEASSPSTGQPSLCSFEINEIYSGCLILEDDIEEPPGAASSLEADGPNQVDELKSMEEELDKMEREACCFGSEDESSSKAETEYSFDDWDWQNGSLSSLSLPESTREAKSNLNNMSTTEEYLISKCVLDLKIMQTIMHENDDRLRNIEQILDEVEMKQKEQEERMSLWATSREFTNAYKLPLAVGPPSLNYIPPVLQLSGGQKPDTSGNYPTLPRFPRMLPTLCDPGKQNTDEQFQCTQGAKDSLETSRIQNTSSQGRPRESTAQAKATQFNSALFTLSSHRQGPSASPSCHWDSTRMSVEPVSSEIYNAESRNKDDGKVHLKWKMEVKEMAKKAATGQLTVPPWHPQSSLTLESEAENEPDALLQPPIRSPENTDWQRVIEYHRENDEPRGNGKFDKTGNNDCDSDQHGRQPRLGSFTSIRHPSPRQKEQPEHSEAFQASSDTLVAVEKSYSTSSPIEEDFEGIQGAFAQPQVSGEEKFQMRKILGKNAEILPRSQFQPVRSTEDEQEETSKESPKELKEKDISLTDIQDLSSISYEPDSSFKEASCKTPKINHAPTSVSTPLSPGSVSSAASQYKDCLESITFQVKTEFASCWNSQEFIQTLSDDFISVRERAKKLDSLLTSSETPPSRLTGLKRLSSFIGAGSPSLVKACDSSPPHATQRRSLPKVEAFSQHHIDELPPPSQELLDDIELLKQQQGSSTVLHENTASDGGGTANDQRHLEEQETDSKKEDSSMLLSKETEDLGEDTERAHSTLDEDLERWLQPPEESVELQDLPKGSERETNIKDQKVGEEKRKREDSITPERRKSEGVLGTSEEDELKSCFWKRLGWSESSRIIVLDQSDLSD

SEQ ID NO: 4 - protein, 1232 aa, mouse; identical to SEQ ID NO: 1

SEQ ID NO: 5 - protein, 1450 aa, mouse
MSRGAPFPVPCPVLLGTFTDDSLEAQLHEYAKQGNCVKLKKILKKGVCVDAVNTQGQSALFVAALLGHVKLVDVLVDYGSDPNHRCFDGSTPVHAAAFSGNQWILSKLLTAGGDLRLHDEKGRNPQAWALTAGKDRSTQMVEFMQRCTSHMKAIIQGFSYDLLKKIDSPQRLIGSPPWFGSLIQGSPNSSPNRQLKPGIISAQNIYSFGFGKFYLTSGMQLTYPGSLPVIGEKEVVQADDEPTFSFFSGPYMVMTNLVWNRSRVTVKELNLPTRPHCSRLRLADLLIAEQEHSSNLRHPNLLQLMAVCLSRDLEKIRLVYERIAVGTLFSVLHERRSQFPVLHMEVIVHLLLQVADALIYLHSRGFIHRSLSSYAVHIVSAGEARLTNLEYLTESQDSGAHRNVTRMPLPTQLYNWAAPEVVLQKAATVKSDIYSFSVIIQEILTDSIPWNGLDGSLVKETIALGNYLEADVRLPEPYYDIVKSGIHAKQKNRTMNLQDIRYILKNDLKEFIGAQKTQPTESPRGQSYEPHPDVNICLGLTSEYQKDPPDLDIKELKEMGSQPHSPTDHSFLTVKPTLAPQTLDSSLSAQKPDNANVPSPPAACLAEEVRSPTASQDSLCSFEINEIYSGCLTLGTDKEEECLGTAASPEGDRPNQGDELPSLEEELDKMERELHCFCEEDKSISEVDTDLLFEDDDWQSDSLGSLNLPEPTREAKGKTSSWSKTDEYVSKCVLNLKISQVMMQQSAEWLRKLEQEVEELEWAQKELDSQCSSLRDASLKFANAKFQPAVGPPSLAYLPPVMQLPGLKQPENGGTWLTLARSPGNEREFQEGHFSKKPEKLSACGWKPFTQVSEESRGDCSELNNQLPTLRGPGKQSTGEQLPSTQEARESLEKNTNQNSRSMASVSSEIYATKSRNNEDNGEAHLKWRLAVKEMAEKAVSGQLLLPPWNPQSSAPFESKVENESTPLPRPPIRGPESTEWQHILEYQRENDEPKGNTKFGKMDNSDCDKNKHSRWTGLQRFTGIRYPFFRNHEQPEQNEASQASCDTSVGTEKFYSTSSPIGDDFERFQDSFAQRQGYVEENFQIREIFEKNAEILTKPQFQAIQCAEDKQDETLGETPKELKEKNTSLTDIQDLSSITYDQDGYFKETSYKTPKLKHAPTSASTPLSPESISSAASHYEDCLENTTFHVKRGSTFCWNGQEAMRTLSAKFTTVRERAKSLESLLASSKSLPAKLTDSKRLCMLSETGSSNVSAAFVTSTHATKRKSLPRELAEATSQQHLDELPPPAQELLDEIEQLKQQQVSSLASHENTARDLSVTNKDKKHLEEQETNSSKDSSFLSSREIQDLEDTERAHSSLDEDLERFLQSPEENTALLDPTKGSTREKKNKDQDVVEQKRKKKESIKPERRESDSSLGTLEEDELKPCFWKRLGWSEPSRIIVLDQSDLSD

SEQ ID NO: 6 - protein, 1450 aa, mouse; identical to SEQ ID NO: 5

SEQ ID NO: 7 - DNA, 4416 bp, mouse
ccactgaagaaagagaaggtgggctcatcatcagcctggaccacttccttctctccaatgactggaagagatcctgggtaagtaagctgcatccctgaggtgagataaaacttcccaaagccaaagctgtagatattttgggcagaaatgattccaggtttaagctgtcggttgggagaactatttgggcttccctgaatgaggctatcatccagggcttctcctatgaccttctgaagaagatagactctcctcagcgactcatcggcagcccaccctggtttggtagcctcattcagggaagcccaaatagttctcccaaccgacagcttaaacctggaatcatttctgcccaaaatatctacagctttggctttgggaagttttatctcacctcagggatgcagcttacttacccaggatctcttccagtcattggagagaaggaagtggtccaggctgatgatgagcccaccttctctttcttcagtggcccctacatggtcatgactaacctcgtgtggaataggagcagagtcacagtaaaggagctgaaccttcccacccgtccccactgtagcaggctgaggttggccgacttgctgattgctgagcaggagcacagcagcaacctgcggcatcctaacctgctgcaactgatggctgtatgtttgtcccgggacctggagaaaattcgcctggtttacgagcgtatcgcagtcggcacactgttcagtgtcctccatgaacgaaggtcccagttcccagtgctgcacatggaggtgattgtgcacctgttgctccaggttgctgatgccttgatatacctgcattcccgggggttcatccaccgctccctcagctcctacgctgtccacatcgtctctgcaggagaagcaaggctgactaacctggaatacctgacggaaagccaggacagtggtgcacacaggaacgtgactcgaatgcccctccccacccagctgtacaactgggctgcaccagaagtggtcttgcagaaggcagccacggtgaagtcagacatatacagcttttccgtgatcatacaagagatcttaacagacagtataccctggaatggcttggatggctcacttgttaaagaaaccatagccttgggaaattatttagaagctgatgtcaggcttccggaaccttactatgatattgttaagtcaggaatccatgccaagcagaagaaccgaacaatgaaccttcaagatattcgttatattctgaagaatgacttaaaggaatttattggagctcagaaaactcagccaaccgagagccccagagggcagagctatgaaccccatcctgatgttaatatctgcctaggtctaacttcagaatatcaaaaggaccctccagacttggacatcaaggaactaaaggaaatgggtagtcagccccattcacctacagatcactcctttctcactgtaaaaccaacactagctcctcagaccctagattcaagtctgtcagcccagaaacctgacaatgcaaatgttccttctcctcctgctgcatgtctggcagaagaggtcaggagccccactgcaagtcaggacagcctctgcagctttgaaatcaatgagatctactcaggctgcttgacactgggaactgacaaggaggaagagtgtctggggactgctgcttcacctgagggggatagaccaaaccagggagatgagctgccatccctggaagaagagctcgataagatggagagagaattgcactgtttttgtgaagaggacaaaagcatttcagaagttgacacagaccttctttttgaggatgatgactggcaaagtgattctcttggttcactcaacctgcccgaaccaaccagagaagccaagggcaaaacgagcagctggtccaagactgatgagtatgtcagtaagtgtgtgctgaatctgaagatttcacaggtgatgatgcagcagagcgctgagtggctgaggaagcttgagcaggaggtagaggagctcgagtgggcacagaaggagctggacagtcagtgcagcagtttgcgggatgcttcattaaagtttgcaaatgccaagttccagccggctgtaggccctccatctttggcctatcttcctcctgttatgcaattaccagggctcaagcagcctgaaaatggtggcacctggttaaccctagcaaggtctccaggaaatgagagagagttccaagagggacattttagcaaaaaacctgagaaactaagtgcctgtggctggaagccttttacacaagtgtctgaagaaagcagaggggactgctcagagctaaacaatcagctgccgactcttcgtggtcctgggaagcagagcacaggtgagcagttaccatccactcaagaagcaagggagagtttggaaaaaaatacaaaccaaaatagtaggagtatggcgtctgtgtcttctgaaatctatgctactaagtcaagaaataatgaggataatggagaggcacacttgaaatggagattggcagtaaaagaaatggcagagaaagcagtttccggacagctcttattacctccttggaatcctcagagtagtgcgccttttgagagtaaggttgaaaatgagagcactcctttgccacggcccccaattagaggtcctgagagcacagaatggcagcacattttagaataccagagggaaaatgatgagcccaaaggaaatacgaagtttggcaaaatggacaacagtgactgtgacaagaacaagcacagcagatggacaggcctccagcgcttcactggtattagatacccattcttcagaaaccacgagcagccagagcagaatgaagcctctcaagcaagctgtgacacgtctgtgggcactgagaagttctacagcacctcaagtcccataggagacgactttgaaagattccaagattcttttgcccaacgtcaaggctatgttgaagaaaatttccaaataagagaaatatttgaaaagaatgctgagattttgaccaagcctcagtttcaagctattcaatgtgctgaagacaaacaagacgaaacattaggggagacgccaaaggaactgaaagagaaaaacacatcactgacagacattcaagacttgtccagcatcacctatgatcaagacggctattttaaggaaacctcatacaaaacacccaaattaaaacacgcaccaactagtgccagtaccccgctaagcccagagtcgatttcttcagctgctagtcactatgaagactgccttgaaaataccacatttcatgttaaaagaggatctacattttgttggaatggccaagaagctatgagaactttgtctgccaaatttacaactgtccgagagagagctaagagcctggaatcacttctcgcttcttctaaaagcctacctgccaagctgactgactccaagagattgtgtatgttgagtgagactggctcttctaacgtttctgcggcatttgtaacatcaactcatgctaccaagaggaagagcctacccagagaactggcagaagccacctctcaacagcatcttgatgagcttccaccaccagctcaggagctacttgatgaaattgagcaactgaagcagcagcaggtctcatccctggcgtcacatgagaacacggcacgtgatctgagtgtcactaacaaggataagaagcatttggaagaacaagaaaccaacagtagtaaagacagcagttttctttccagcagagaaattcaggatctggaagatacagagagagctcattcttctcttgatgaggacctggaaagattcctgcagtcacctgaggagaacacggcactgctggaccctaccaagggctctacaagggagaaaaaaaacaaagatcaagacgttgttgagcagaagagaaaaaagaaagaaagcatcaagccagagagaagggagtcagacagctccctagggaccttggaagaagatgaactaaaaccctgtttttggaagcgactgggttggtccgaaccttccaggataattgtgctggatcagagcgacttgtcagactgattggaactggaccgtgcaagcattgtggctgtggcctcctttcttctcttatctgcttcagttgcctcaaggacagtagtttcagtctgtaactcacactttgttctgctgctactatgggcacaataatgtgtccctatcatgtgtagcatgcttaatcatttgttttaaatcaggtttctgaaaagtgcaaagtaaccatagtggcaaacattttgtgttcagaatggcttttgttttttctgatgtaaatttttgaaaccataactttgttatataataaagtgtttactttcaatgctaaaaaaaaaaaaaa

SEQ ID NO: 8 - DNA, 4945 bp, human
atcagtccgcattccccgcacccacccacgtcttcccgggagtcgtatcccgagcatggaggttactgagaccgttatttcttcatggcctgcctagcttaagcagtagctggaaaagatgtctcgggctgttcgtcttccagtcccctgtcctgttcaacttggtaccttaagaaatgactccctggaagctcagcttcatgagtatgtcaaacaagggaactatgtgaaagtgaagaaaattcttaagaaaggaatttatgttgatgcagttaactccttgggccaaacagcactttttgttgcggcgttattgggccttaggaaattcgttgatgttctggtggattatggatcagatccaaatcaccgctgctttgatgggagcacccctgtccatgcagcagcattttcgggcaatcagtggatccttagcaaactgctggatgcaggaggtgacctgcgactccacgatgagaggggtcaaaacccgaagacttgggctttgacagcaggaaaggagcgtagcacccagatagtggagttcatgcagcgctgtgcctcacacatgcaggccatcatccagggcttctcttacgacctcctgaagaagatagactccccgcagcggcttgtctacagcccgtcctggtgtgggggcctcgtgcagggaaaccctaatggctctcctaaccgactgcttaaagctggagtcatttctgctcaaaatatctacagctttggttttgggaagttttatcttactggggcgacacagatggcctatctaggatctcttccggtcattggagaaaaggaagtgattcaagctgatgatgagcccaccttctctttcttcagcggcccctacatggtcatgaccaacctagtgtggaatgggagcagggtcacagtgaaagagctgaatctccccacccacccacactgcagcaggctgcggctggccgacttgttaattgccgagcaggaacacagcagcaagctgcggcacccctacttgctacagttgatggctgtgtgtctctcccaggacctagagaaaacccgccttgtgtacgagcgcatcactatcggcacattgttcagtgtccttcatgaacgacggtcccagttcccagtgctgcacatggaggtgattgtgcacctgctgctccagatatctgatgccctgagatacctgcatttccaggggtttatccaccgctccctcagctcctatgctgtccatatcatctccccaggtgaagcgaggctgaccaacctggagtacatgttggaaagcgaggacagaggtgtacagagggacctgactcgagtgccccttcctacgcagctatacaactgggccgcaccagaagtgatcttacagaaggcagccacagtgaaatcagacatctacagcttttctatgatcatgcaggagattttaacagatgacataccctggaagggcttagatggctcagttgttaaaaaagccgtagtctcggggaattatttagaagctgatgtcaggcttccgaaaccttactatgatattgttaagtcaggcatccacgtcaagcagaaagaccgaactatgaaccttcaagatatccggtatattctgaagaatgacttaaaggattttactggagcccagagaactcaaccaaccgagagccccagagtgcagagatacggactccatcccgatgtcaatgtctatctaggactgacttcagaacaccccagagagacacctgacatggaaatcatagaactaaaggaaatgggcagtcaacctcattcaccaagggttcactctttattcactgaggggacactagatcctcaggccccagatccatgtctgatggccagggagactcagaatcaagatgctccttgccctgctccatttatggcagaagaggccagcagccccagcacaggtcagccaagcctctgcagtttcgaaatcaacgagatctactcaggctgcttgattttggaagatgacatagaagagcctccaggagctgcttcatctttggaggcagacggacctaaccaggtagatgaactgaaatccatggaagaagagctggataagatggagagagaggcgtgttgttttggcagtgaggatgagagctcttcaaaagctgagacagagtactcttttgatgactgggactggcaaaacggttcactcagttcactcagccttcctgagtcaaccagagaagccaagagcaatttgaacaacatgtccacgactgaggagtatctcatcagtaagtgtgtgctggatctaaagattatgcagacaataatgcacgagaatgatgataggctgaggaatatcgagcagatattagatgaagtcgagatgaaacagaaggaacaggaagagcgcatgtctttatgggccacttcaagagagtttacaaatgcctacaagttacctctggccgtgggccctccatctttaaactatattcctcctgtcctacagctttcagggggtcagaagccagacaccagtggcaactacccaaccctaccaagatttccaagaatgctgccgactctttgtgaccctggaaaacagaacacagatgaacaatttcagtgcactcaaggagccaaggacagtttggaaacaagcaggatccaaaataccagtagccagggaagacctagagagtccactgcccaagccaaagccacacagtttaatagtgcactcttcactctgtcaagccaccggcagggaccttctgcatcacccagctgtcactgggactctaccaggatgagtgtggaacctgtttcttctgaaatctataatgcagagtccagaaataaagatgatggaaaggtacacttaaaatggaaaatggaggtgaaagaaatggcaaagaaagcagctactggacagctcacagtacctccttggcatcctcagagtagtctgactttagagagcgaggctgaaaatgagcccgacgccctgctgcagccccccattaggagcccagaaaacacggattggcagcgagttattgagtatcatagggaaaatgatgagcccagaggaaatggcaagtttgacaagacgggcaacaatgactgtgacagtgaccagcatggcagacagcccaggcttggaagcttcaccagtatcaggcacccatctcccagacaaaaggagcaaccagagcatagtgaagccttccaagcaagttctgacacattggtggctgtagagaaatcttacagtcatcagtccatgcaatcaacttgttcaccagagtcttctgaggatataacagatgaatttttaactccagacggtgaatatttttactcctcgactgctcaagaaaacttagctctagagacctcgagtcccatagaagaggactttgaaggaatacaaggtgcatttgcccaacctcaagtctctggtgaggaaaagttccaaatgagaaaaattcttggaaagaatgctgagattttgcccaggtctcaatttcaacctgtacgaagtactgaagatgaacaagaagagacatcaaaggagtcaccaaaggaactgaaagagaaagacatatcattgacggatattcaagacctgtctagtatctcctatgaaccagacagctcttttaaggaagcttcatgcaaaacacccaaaataaaccatgcacctaccagtgtcagcactccactcagcccagggtccgtttcttcagctgccagtcagtataaagactgccttgaaagtatcacatttcaggttaagacagagtttgcctcttgctggaacagtcaagaatttattcaaactttgtctgatgactttataagtgtccgagagagagcaaagaaactggattctctccttacttcctctgaaactcccccttcaagactgactggtcttaaaagattgtcttcatttattggggctggatcccccagccttgttaaggcatgtgactcatcaccaccccatgccacccagagaaggagcctgcctaaagtagaagccttctcacagcatcacattgatgagctgccaccaccatctcaggagctacttgatgacattgagctcttgaaacagcagcagggctcatccacggtgttgcatgagaacacagcaagtgatggaggaggcactgcaaatgatcaaaggcacttagaagaacaagaaactgacagtaaaaaagaagatagtagtatgcttttgtccaaagaaactgaagatcttggagaggacacagagagagctcactctactctggatgaggacctggaaagatggctgcagccacctgaggagagcgtggagctacaagaccttcccaagggctctgaaagggagacaaatatcaaagatcaaaaagttggtgaagagaaaagaaaaagggaagatagcattacaccagaaagaaggaaatcagagggtgttctagggacttctgaagaagatgaactaaaatcctgtttttggaagcgactaggttggtccgaatcatccaggataatcgtgctggatcagagtgacttgtcagactgattggaattggatcatagacggactcctggcctgagtttgagtgtcctggttgtaagctcctttcttctctttctgcttcagttgctgtcagggcagcagttccagttctgtaagtctcactttgttcagctgccacaatagacatcatcgtttggccctctctgttagcagcacattcaaccatttgttttcagtcagatttctgaaaagtgagaggtagttttgatagtaaaaatttttggttgtgcctagaatggctttggttttgttgatgttaattttcaaaaactttaactcttgttatataataaaatgtttaattttaataacagaaaaaaaaaaaaaaaaaaa

SEQ ID NO: 9 - DNA, 4825 bp, human
atcagtccgcattccccgcacccacccacgtcttcccgggagtcgtatcccgagcatggaggttactgagaccgttatttcttcatggcctgcctagcttaagcagtagctggaaaagatgtctcgggctgttcgtcttccagtcccctgtcctgttcaacttggtaccttaagaaatgactccctggaagctcagcttcatgagtatgtcaaacaagggaactatgtgaaagtgaagaaaattcttaagaaaggaatttatgttgatgcagttaactccttgggccaaacagcactttttgttgcggcgttattgggccttaggaaattcgttgatgttctggtggattatggatcagatccaaatcaccgctgctttgatgggagcacccctgtccatgcagcagcattttcgggcaatcagtggatccttagcaaactgctggatgcaggaggtgacctgcgactccacgatgagaggggtcaaaacccgaagacttgggctttgacagcaggaaaggagcgtagcacccagatagtggagttcatgcagcgctgtgcctcacacatgcaggccatcatccagggcttctcttacgacctcctgaagaagatagactccccgcagcggcttgtctacagcccgtcctggtgtgggggcctcgtgcagggaaaccctaatggctctcctaaccgactgcttaaagctggagtcatttctgctcaaaatatctacagctttggttttgggaagttttatcttactggggcgacacagatggcctatctaggatctcttccggtcattggagaaaaggaagtgattcaagctgatgatgagcccaccttctctttcttcagcggcccctacatggtcatgaccaacctagtgtggaatgggagcagggtcacagtgaaagagctgaatctccccacccacccacactgcagcaggctgcggctggccgacttgttaattgccgagcaggaacacagcagcaagctgcggcacccctacttgctacagttgatggctgtgtgtctctcccaggacctagagaaaacccgccttgtgtacgagcgcatcactatcggcacattgttcagtgtccttcatgaacgacggtcccagttcccagtgctgcacatggaggtgattgtgcacctgctgctccagatatctgatgccctgagatacctgcatttccaggggtttatccaccgctccctcagctcctatgctgtccatatcatctccccaggtgaagcgaggctgaccaacctggagtacatgttggaaagcgaggacagaggtgtacagagggacctgactcgagtgccccttcctacgcagctatacaactgggccgcaccagaagtgatcttacagaaggcagccacagtgaaatcagacatctacagcttttctatgatcatgcaggagattttaacagatgacataccctggaagggcttagatggctcagttgttaaaaaagccgtagtctcggggaattatttagaagctgatgtcaggcttccgaaaccttactatgatattgttaagtcaggcatccacgtcaagcagaaagaccgaactatgaaccttcaagatatccggtatattctgaagaatgacttaaaggattttactggagcccagagaactcaaccaaccgagagccccagagtgcagagatacggactccatcccgatgtcaatgtctatctaggactgacttcagaacaccccagagagacacctgacatggaaatcatagaactaaaggaaatgggcagtcaacctcattcaccaagggttcactctttattcactgaggggacactagatcctcaggccccagatccatgtctgatggccagggagactcagaatcaagatgctccttgccctgctccatttatggcagaagaggccagcagccccagcacaggtcagccaagcctctgcagtttcgaaatcaacgagatctactcaggctgcttgattttggaagatgacatagaagagcctccaggagctgcttcatctttggaggcagacggacctaaccaggtagatgaactgaaatccatggaagaagagctggataagatggagagagaggcgtgttgttttggcagtgaggatgagagctcttcaaaagctgagacagagtactcttttgatgactgggactggcaaaacggttcactcagttcactcagccttcctgagtcaaccagagaagccaagagcaatttgaacaacatgtccacgactgaggagtatctcatcagtaagtgtgtgctggatctaaagattatgcagacaataatgcacgagaatgatgataggctgaggaatatcgagcagatattagatgaagtcgagatgaaacagaaggaacaggaagagcgcatgtctttatgggccacttcaagagagtttacaaatgcctacaagttacctctggccgtgggccctccatctttaaactatattcctcctgtcctacagctttcagggggtcagaagccagacaccagtggcaactacccaaccctaccaagatttccaagaatgctgccgactctttgtgaccctggaaaacagaacacagatgaacaatttcagtgcactcaaggagccaaggacagtttggaaacaagcaggatccaaaataccagtagccagggaagacctagagagtccactgcccaagccaaagccacacagtttaatagtgcactcttcactctgtcaagccaccggcagggaccttctgcatcacccagctgtcactgggactctaccaggatgagtgtggaacctgtttcttctgaaatctataatgcagagtccagaaataaagatgatggaaaggtacacttaaaatggaaaatggaggtgaaagaaatggcaaagaaagcagctactggacagctcacagtacctccttggcatcctcagagtagtctgactttagagagcgaggctgaaaatgagcccgacgccctgctgcagccccccattaggagcccagaaaacacggattggcagcgagttattgagtatcatagggaaaatgatgagcccagaggaaatggcaagtttgacaagacgggcaacaatgactgtgacagtgaccagcatggcagacagcccaggcttggaagcttcaccagtatcaggcacccatctcccagacaaaaggagcaaccagagcatagtgaagccttccaagcaagttctgacacattggtggctgtagagaaatcttacagtacctcgagtcccatagaagaggactttgaaggaatacaaggtgcatttgcccaacctcaagtctctggtgaggaaaagttccaaatgagaaaaattcttggaaagaatgctgagattttgcccaggtctcaatttcaacctgtacgaagtactgaagatgaacaagaagagacatcaaaggagtcaccaaaggaactgaaagagaaagacatatcattgacggatattcaagacctgtctagtatctcctatgaaccagacagctcttttaaggaagcttcatgcaaaacacccaaaataaaccatgcacctaccagtgtcagcactccactcagcccagggtccgtttcttcagctgccagtcagtataaagactgccttgaaagtatcacatttcaggttaagacagagtttgcctcttgctggaacagtcaagaatttattcaaactttgtctgatgactttataagtgtccgagagagagcaaagaaactggattctctccttacttcctctgaaactcccccttcaagactgactggtcttaaaagattgtcttcatttattggggctggatcccccagccttgttaaggcatgtgactcatcaccaccccatgccacccagagaaggagcctgcctaaagtagaagccttctcacagcatcacattgatgagctgccaccaccatctcaggagctacttgatgacattgagctcttgaaacagcagcagggctcatccacggtgttgcatgagaacacagcaagtgatggaggaggcactgcaaatgatcaaaggcacttagaagaacaagaaactgacagtaaaaaagaagatagtagtatgcttttgtccaaagaaactgaagatcttggagaggacacagagagagctcactctactctggatgaggacctggaaagatggctgcagccacctgaggagagcgtggagctacaagaccttcccaagggctctgaaagggagacaaatatcaaagatcaaaaagttggtgaagagaaaagaaaaagggaagatagcattacaccagaaagaaggaaatcagagggtgttctagggacttctgaagaagatgaactaaaatcctgtttttggaagcgactaggttggtccgaatcatccaggataatcgtgctggatcagagtgacttgtcagactgattggaattggatcatagacggactcctggcctgagtttgagtgtcctggttgtaagctcctttcttctctttctgcttcagttgctgtcagggcagcagttccagttctgtaagtctcactttgttcagctgccacaatagacatcatcgtttggccctctctgttagcagcacattcaaccatttgttttcagtcagatttctgaaaagtgagaggtagttttgatagtaaaaatttttggttgtgcctagaatggctttggttttgttgatgttaattttcaaaaactttaactcttgttatataataaaatgtttaattttaataacagaaaaaaaaaaaaaaaaaaa

SEQ ID NO: 10 - DNA, 4416 bp, mouse; identical to SEQ ID NO: 7

SEQ ID NO: 11 - DNA, 4776 bp, mouse
acagcttccggtgggtgaccccgggactccggagctccctcagagtgtggcggtttctgagcattcatgtcggcaggcctgcctagagtaagaagtagctggaaaacatgtctcggggggctccttttccagtcccttgcccggtgctgctggggaccttcacagatgactccctggaggctcagcttcacgaatacgccaagcaagggaactgcgtgaagctcaagaagattcttaagaagggagtctgtgtcgatgcagttaacactcagggccagtcagcgctctttgttgcagcattactgggccatgtgaaattagtggatgttctggtggattatggatcagatccaaatcaccgctgctttgacgggagcactcctgtccatgcagcagccttttcaggcaaccagtggatcctcagtaaactgctgactgcagggggtgacctgcgactccatgatgagaagggtcggaatccacaggcctgggccttgacagcagggaaggatcgtagcactcagatggtggagttcatgcaacgttgtacttcacatatgaaggctatcatccagggcttctcctatgaccttctgaagaagatagactctcctcagcgactcatcggcagcccaccctggtttggtagcctcattcagggaagcccaaatagttctcccaaccgacagcttaaacctggaatcatttctgcccaaaatatctacagctttggctttgggaagttttatctcacctcagggatgcagcttacttacccaggatctcttccagtcattggagagaaggaagtggtccaggctgatgatgagcccaccttctctttcttcagtggcccctacatggtcatgactaacctcgtgtggaataggagcagagtcacagtaaaggagctgaaccttcccacccgtccccactgtagcaggctgaggttggccgacttgctgattgctgagcaggagcacagcagcaacctgcggcatcctaacctgctgcaactgatggctgtatgtttgtcccgggacctggagaaaattcgcctggtttacgagcgtatcgcagtcggcacactgttcagtgtcctccatgaacgaaggtcccagttcccagtgctgcacatggaggtgattgtgcacctgttgctccaggttgctgatgccttgatatacctgcattcccgggggttcatccaccgctccctcagctcctacgctgtccacatcgtctctgcaggagaagcaaggctgactaacctggaatacctgacggaaagccaggacagtggtgcacacaggaacgtgactcgaatgcccctccccacccagctgtacaactgggctgcaccagaagtggtcttgcagaaggcagccacggtgaagtcagacatatacagcttttccgtgatcatacaagagatcttaacagacagtataccctggaatggcttggatggctcacttgttaaagaaaccatagccttgggaaattatttagaagctgatgtcaggcttccggaaccttactatgatattgttaagtcaggaatccatgccaagcagaagaaccgaacaatgaaccttcaagatattcgttatattctgaagaatgacttaaaggaatttattggagctcagaaaactcagccaaccgagagccccagagggcagagctatgaaccccatcctgatgttaatatctgcctaggtctaacttcagaatatcaaaaggaccctccagacttggacatcaaggaactaaaggaaatgggtagtcagccccattcacctacagatcactcctttctcactgtaaaaccaacactagctcctcagaccctagattcaagtctgtcagcccagaaacctgacaatgcaaatgttccttctcctcctgctgcatgtctggcagaagaggtcaggagccccactgcaagtcaggacagcctctgcagctttgaaatcaatgagatctactcaggctgcttgacactgggaactgacaaggaggaagagtgtctggggactgctgcttcacctgagggggatagaccaaaccagggagatgagctgccatccctggaagaagagctcgataagatggagagagaattgcactgtttttgtgaagaggacaaaagcatttcagaagttgacacagaccttctttttgaggatgatgactggcaaagtgattctcttggttcactcaacctgcccgaaccaaccagagaagccaagggcaaaacgagcagctggtccaagactgatgagtatgtcagtaagtgtgtgctgaatctgaagatttcacaggtgatgatgcagcagagcgctgagtggctgaggaagcttgagcaggaggtagaggagctcgagtgggcacagaaggagctggacagtcagtgcagcagtttgcgggatgcttcattaaagtttgcaaatgccaagttccagccggctgtaggccctccatctttggcctatcttcctcctgttatgcaattaccagggctcaagcagcctgaaaatggtggcacctggttaaccctagcaaggtctccaggaaatgagagagagttccaagagggacattttagcaaaaaacctgagaaactaagtgcctgtggctggaagccttttacacaagtgtctgaagaaagcagaggggactgctcagagctaaacaatcagctgccgactcttcgtggtcctgggaagcagagcacaggtgagcagttaccatccactcaagaagcaagggagagtttggaaaaaaatacaaaccaaaatagtaggagtatggcgtctgtgtcttctgaaatctatgctactaagtcaagaaataatgaggataatggagaggcacacttgaaatggagattggcagtaaaagaaatggcagagaaagcagtttccggacagctcttattacctccttggaatcctcagagtagtgcgccttttgagagtaaggttgaaaatgagagcactcctttgccacggcccccaattagaggtcctgagagcacagaatggcagcacattttagaataccagagggaaaatgatgagcccaaaggaaatacgaagtttggcaaaatggacaacagtgactgtgacaagaacaagcacagcagatggacaggcctccagcgcttcactggtattagatacccattcttcagaaaccacgagcagccagagcagaatgaagcctctcaagcaagctgtgacacgtctgtgggcactgagaagttctacagcacctcaagtcccataggagacgactttgaaagattccaagattcttttgcccaacgtcaaggctatgttgaagaaaatttccaaataagagaaatatttgaaaagaatgctgagattttgaccaagcctcagtttcaagctattcaatgtgctgaagacaaacaagacgaaacattaggggagacgccaaaggaactgaaagagaaaaacacatcactgacagacattcaagacttgtccagcatcacctatgatcaagacggctattttaaggaaacctcatacaaaacacccaaattaaaacacgcaccaactagtgccagtaccccgctaagcccagagtcgatttcttcagctgctagtcactatgaagactgccttgaaaataccacatttcatgttaaaagaggatctacattttgttggaatggccaagaagctatgagaactttgtctgccaaatttacaactgtccgagagagagctaagagcctggaatcacttctcgcttcttctaaaagcctacctgccaagctgactgactccaagagattgtgtatgttgagtgagactggctcttctaacgtttctgcggcatttgtaacatcaactcatgctaccaagaggaagagcctacccagagaactggcagaagccacctctcaacagcatcttgatgagcttccaccaccagctcaggagctacttgatgaaattgagcaactgaagcagcagcaggtctcatccctggcgtcacatgagaacacggcacgtgatctgagtgtcactaacaaggataagaagcatttggaagaacaagaaaccaacagtagtaaagacagcagttttctttccagcagagaaattcaggatctggaagatacagagagagctcattcttctcttgatgaggacctggaaagattcctgcagtcacctgaggagaacacggcactgctggaccctaccaagggctctacaagggagaaaaaaaacaaagatcaagacgttgttgagcagaagagaaaaaagaaagaaagcatcaagccagagagaagggagtcagacagctccctagggaccttggaagaagatgaactaaaaccctgtttttggaagcgactgggttggtccgaaccttccaggataattgtgctggatcagagcgacttgtcagactgattggaactggaccgtgcaagcattgtggctgtggcctcctttcttctcttatctgcttcagttgcctcaaggacagtagtttcagtctgtaactcacactttgttctgctgctactatgggcacaataatgtgtccctatcatgtgtagcatgcttaatcatttgttttaaatcaggtttctgaaaagtgcaaagtaaccatagtggcaaacattttgtgttcagaatggcttttgttttttctgatgtaaatttttgaaaccataactttgttatataataaagtgtttactttcaatgctaaaaaaaaaaaaaa

SEQ ID NO: 12 - DNA, 4795 bp, mouse
acacacctttaatcccagcatttgggaggcagagacaggcagatctgtgttccaggccatgataagttccacaacagccagaactacataatgagaccctgtctacaaacaaacaaacaaaacaacatgtctcggggggctccttttccagtcccttgcccggtgctgctggggaccttcacagatgactccctggaggctcagcttcacgaatacgccaagcaagggaactgcgtgaagctcaagaagattcttaagaagggagtctgtgtcgatgcagttaacactcagggccagtcagcgctctttgttgcagcattactgggccatgtgaaattagtggatgttctggtggattatggatcagatccaaatcaccgctgctttgacgggagcactcctgtccatgcagcagccttttcaggcaaccagtggatcctcagtaaactgctgactgcagggggtgacctgcgactccatgatgagaagggtcggaatccacaggcctgggccttgacagcagggaaggatcgtagcactcagatggtggagttcatgcaacgttgtacttcacatatgaaggctatcatccagggcttctcctatgaccttctgaagaagatagactctcctcagcgactcatcggcagcccaccctggtttggtagcctcattcagggaagcccaaatagttctcccaaccgacagcttaaacctggaatcatttctgcccaaaatatctacagctttggctttgggaagttttatctcacctcagggatgcagcttacttacccaggatctcttccagtcattggagagaaggaagtggtccaggctgatgatgagcccaccttctctttcttcagtggcccctacatggtcatgactaacctcgtgtggaataggagcagagtcacagtaaaggagctgaaccttcccacccgtccccactgtagcaggctgaggttggccgacttgctgattgctgagcaggagcacagcagcaacctgcggcatcctaacctgctgcaactgatggctgtatgtttgtcccgggacctggagaaaattcgcctggtttacgagcgtatcgcagtcggcacactgttcagtgtcctccatgaacgaaggtcccagttcccagtgctgcacatggaggtgattgtgcacctgttgctccaggttgctgatgccttgatatacctgcattcccgggggttcatccaccgctccctcagctcctacgctgtccacatcgtctctgcaggagaagcaaggctgactaacctggaatacctgacggaaagccaggacagtggtgcacacaggaacgtgactcgaatgcccctccccacccagctgtacaactgggctgcaccagaagtggtcttgcagaaggcagccacggtgaagtcagacatatacagcttttccgtgatcatacaagagatcttaacagacagtataccctggaatggcttggatggctcacttgttaaagaaaccatagccttgggaaattatttagaagctgatgtcaggcttccggaaccttactatgatattgttaagtcaggaatccatgccaagcagaagaaccgaacaatgaaccttcaagatattcgttatattctgaagaatgacttaaaggaatttattggagctcagaaaactcagccaaccgagagccccagagggcagagctatgaaccccatcctgatgttaatatctgcctaggtctaacttcagaatatcaaaaggaccctccagacttggacatcaaggaactaaaggaaatgggtagtcagccccattcacctacagatcactcctttctcactgtaaaaccaacactagctcctcagaccctagattcaagtctgtcagcccagaaacctgacaatgcaaatgttccttctcctcctgctgcatgtctggcagaagaggtcaggagccccactgcaagtcaggacagcctctgcagctttgaaatcaatgagatctactcaggctgcttgacactgggaactgacaaggaggaagagtgtctggggactgctgcttcacctgagggggatagaccaaaccagggagatgagctgccatccctggaagaagagctcgataagatggagagagaattgcactgtttttgtgaagaggacaaaagcatttcagaagttgacacagaccttctttttgaggatgatgactggcaaagtgattctcttggttcactcaacctgcccgaaccaaccagagaagccaagggcaaaacgagcagctggtccaagactgatgagtatgtcagtaagtgtgtgctgaatctgaagatttcacaggtgatgatgcagcagagcgctgagtggctgaggaagcttgagcaggaggtagaggagctcgagtgggcacagaaggagctggacagtcagtgcagcagtttgcgggatgcttcattaaagtttgcaaatgccaagttccagccggctgtaggccctccatctttggcctatcttcctcctgttatgcaattaccagggctcaagcagcctgaaaatggtggcacctggttaaccctagcaaggtctccaggaaatgagagagagttccaagagggacattttagcaaaaaacctgagaaactaagtgcctgtggctggaagccttttacacaagtgtctgaagaaagcagaggggactgctcagagctaaacaatcagctgccgactcttcgtggtcctgggaagcagagcacaggtgagcagttaccatccactcaagaagcaagggagagtttggaaaaaaatacaaaccaaaatagtaggagtatggcgtctgtgtcttctgaaatctatgctactaagtcaagaaataatgaggataatggagaggcacacttgaaatggagattggcagtaaaagaaatggcagagaaagcagtttccggacagctcttattacctccttggaatcctcagagtagtgcgccttttgagagtaaggttgaaaatgagagcactcctttgccacggcccccaattagaggtcctgagagcacagaatggcagcacattttagaataccagagggaaaatgatgagcccaaaggaaatacgaagtttggcaaaatggacaacagtgactgtgacaagaacaagcacagcagatggacaggcctccagcgcttcactggtattagatacccattcttcagaaaccacgagcagccagagcagaatgaagcctctcaagcaagctgtgacacgtctgtgggcactgagaagttctacagcacctcaagtcccataggagacgactttgaaagattccaagattcttttgcccaacgtcaaggctatgttgaagaaaatttccaaataagagaaatatttgaaaagaatgctgagattttgaccaagcctcagtttcaagctattcaatgtgctgaagacaaacaagacgaaacattaggggagacgccaaaggaactgaaagagaaaaacacatcactgacagacattcaagacttgtccagcatcacctatgatcaagacggctattttaaggaaacctcatacaaaacacccaaattaaaacacgcaccaactagtgccagtaccccgctaagcccagagtcgatttcttcagctgctagtcactatgaagactgccttgaaaataccacatttcatgttaaaagaggatctacattttgttggaatggccaagaagctatgagaactttgtctgccaaatttacaactgtccgagagagagctaagagcctggaatcacttctcgcttcttctaaaagcctacctgccaagctgactgactccaagagattgtgtatgttgagtgagactggctcttctaacgtttctgcggcatttgtaacatcaactcatgctaccaagaggaagagcctacccagagaactggcagaagccacctctcaacagcatcttgatgagcttccaccaccagctcaggagctacttgatgaaattgagcaactgaagcagcagcaggtctcatccctggcgtcacatgagaacacggcacgtgatctgagtgtcactaacaaggataagaagcatttggaagaacaagaaaccaacagtagtaaagacagcagttttctttccagcagagaaattcaggatctggaagatacagagagagctcattcttctcttgatgaggacctggaaagattcctgcagtcacctgaggagaacacggcactgctggaccctaccaagggctctacaagggagaaaaaaaacaaagatcaagacgttgttgagcagaagagaaaaaagaaagaaagcatcaagccagagagaagggagtcagacagctccctagggaccttggaagaagatgaactaaaaccctgtttttggaagcgactgggttggtccgaaccttccaggataattgtgctggatcagagcgacttgtcagactgattggaactggaccgtgcaagcattgtggctgtggcctcctttcttctcttatctgcttcagttgcctcaaggacagtagtttcagtctgtaactcacactttgttctgctgctactatgggcacaataatgtgtccctatcatgtgtagcatgcttaatcatttgttttaaatcaggtttctgaaaagtgcaaagtaaccatagtggcaaacattttgtgttcagaatggcttttgttttttctgatgtaaatttttgaaaccataactttgttatataataaagtgtttactttcaatgctaaaaaaaaaaaaaa

SEQ ID NO: 13 - DNA, 3348 bp, Human
gagcagcctacaacttcacaaccagaaaccactacccctcaggggttgctttcagataaagatgacatgggaaggagaaatgctggcatagatttcggatccagaaaagcatcagcagcacagcccatacctgaaaacatggacaattccatggttagtgatccacaaccataccatgaagatgcagcttctggagctgagaagacagaagccagagcttctctctcactgatggtggaaagcctttctacaacccaagaggaggccattctctcagtagcagcagaggctcaggtgtttatgaatccttctcatatccagttagaagatcaagaagctttcagctttgatttacaaaaggcccaatccaaaatggagtcagcccaggatgttcaaactatctgcaaagaaaagccttctggaaatgttcaccagacctttacagcaagtgttttgggtatgacaagtactacagccaaaggagatgtttatgccaagactctgcctcccagaagcctttttcagtcctcaaggaagcctgatgctgaagaagtctcctcagattcagagaatattcctgaggagggggatggttctgaagaactggctcatggtcactcttcccagtccttggggaagtttgaagatgaacaagaagtcttctcagaatcaaaaagttttgttgaggacttgagcagctctgaggaggagctggacctcagatgcctctcccaggctttagaggagcctgaagatgcagaagtcttcacagaatcaagcagttatgttgaaaagtacaacacttctgatgattgcagcagctcagaggaagacctgcctctcagacaccctgctcaggccttgggaaagcccaaaaaccaacaagaagtctcctctgcttcaaataatactcctgaagagcagaatgattttatgcagcagctgccttccagatgcccttctcagcccattatgaatcctactgttcagcaacaagtccccaccagttcagtgggcacttctataaaacagagcgattccgtggagccaatccctccaagacaccctttccagccatgggtgaaccctaaagtggagcaagaagtttcctcatctccaaagagcatggctgttgaagagagcatttctatgaagcctctgcctcctaaacttctttgccagcccttgatgaatcctaaagttcaacaaaacatgttctcaggttcagaggacattgctgttgagagagtcatttctgtggagccactactccccagatattctcctcagtccttgacagatcctcaaatccggcaaatctcagaaagcacagctgttgaggaaggcacttatgtggaaccgctgcctcccagatgcctttcccagccctcggagaggcctaagttcctggactcaatgagtacttctgcagaatggagcagtcctgtggcaccaacaccttccaaatacacttccccgccatgggtgacccctaaatttgaggaactgtatcaactctctgcacatccagaaagcactactgttgaagaggacatttctaaggagcagctgcttcccagacatctttcccagttgactgtgggaaataaagtccagcaactgtcctcaaatttcgagcgggctgctattgaggcagacatttctgggagtccattgcctccccaatatgctacccagttcttaaagaggtctaaagttcaggaaatgacctcacgactagagaaaatggctgttgaaggcacttctaacaaatcaccgattcccaggcgtccgacccagtcattcgtgaaatttatggcacagcaaatcttttcagagagctctgctcttaagaggggcagtgatgtggcacctctgcctcccaatcttccttccaaatctttatcaaagcctgaagtcaagcaccaagttttctcagattcagggagtgctaatcctaagggaggcatttcttcaaagatgctacctatgaagcaccctttacagtccttggggaggcctgaagacccacagaaagttttctcttattcagagagagctcctgggaagtgcagcagttttaaagagcagctgtctcccaggcagctttcccaggccttgaggaaacctgagtatgagcaaaaagtctcccctgtttctgccagttctcctaaagagtggaggaattctaaaaagcagctgcctcccaaacattcttcccaagcctcagataggtctaaattccagccacagatgtcatcaaagggcccagtgaatgtacctgtaaagcagagcagcggtgagaagcacctgccttcaagtagtcctttccagcaacaggttcattcaagttctgtgaatgctgctgctaggcgatctgtttttgagagcaattctgacaattggttcctaggaagagatgaagcttttgcaatcaaaaccaagaaattcagccaaggttccaaaaaccccataaagagcattccagcccctgctaccaaacctgggaagttcaccattgctcctgtcaggcaaacatccacttctgggggcatttactctaagaaagaagatcttgagagtggtgatggtaataataaccagcatgcaaacctatccaatcaggatgatgttgaaaagctttttggagttcgactgaaaagagcccctccctcgcagaagtataagagtgagaaacaagataacttcacccagcttgcttcagtgccctcgggcccaatttcatcctctgtaggcaggggacataaaatcagaagcacttcccaggggctcctggatgctgcagggaacctcaccaaaatatcttacgttgcagataagcaacagagcaggcccaaatctgaaagcatggccaagaagcaacctgcttgcaagaccccaggaaagcctgctggtcaacagtcagattatgctgtctcagagccggtttggataactatggcaaagcagaagcagaagagtttcaaggcccacatttctgtgaaagagctgaaaactaagagcaatgctggagccgatgctgagactaaggagcctaaatatgagggagctggctctgcaaatgaaaaccaacctaaaaagatgttcacttccagtgtccataaacaggagaagacagcacagatgaagccacctaagcctacaaaatcagttggatttgaagctcagaagatactgcaagttcctgccatggaaaaagaaaccaaacgatcttcaactctcccagccaagttccagaacccagttgagccaattgagcctgtctggttctcactggccaggaagaaagccaaagcatggagccacatggcagaaatcacgcaataa

SEQ ID NO: 14 - protein, 1381 aa, Mouse
QPTTTEAEVTTVQKMPSDKGDVERELADIDVEAQKEPAPQPTSTDVAESMISGPSPHCEDKKKKDKVGVLPWIKKSTSQKEVTVLRKEEVQVHVHPSRVCGEGEEASCPELQRVQPQMKVSLESTTYHKEKHPRSGLPVPSTSISSATAEDDVSPKMNLPLPLRRRPNTGETSSDSKSTSEYESSSEMQLSPAHSFKPTRKPKDDAGAGTADDEEDGDDEKEEKDNDDDDEENVFLKSENVDVEVSKMEKQQALRYSSKLLGKPKAREASAKLESSSEDERSCEEMTSAHSSQSLEEFEECSESRGFIRGSISEERPATRCHSQALQELEEKEVSTESSSYIEKYESSEDLSSSEQEQQVPPVSKSSLKQWSAPAKPVHPIQTAQQQPPAMVNISGGPKKSGEPLSPTQTVKSRLRSQSECQVLAEPEGVVAADWDILMQPPPRPRLEQEVSAGPELTVFEKSIAVQPSGYPPQPSVKPSIKKEISLGAESAPLESPPPQHHFQPLLKPFVKQQVSAFERVISMDPKLPTQFQPRMKPQGKQSFSSTPESTAFEGSTSVDHMTLTLSQPMLQPYQEVPLESETVAAKMISTGQLHSKYSAHPLSSHQSQPVPESTSAEGEELLPSQPSVTPKFQPLMTQGSIPSAWAIPIYSPAPRMDSKPLMGSAIALESKPSRYSLQPWQSTPFEQVSVTPDHDPAAAAASWSPPIDPPTSRIPSQPLMRLAVKQPTCTELASVSVSQSSSLERLSSRFPFQPRADPEHYGAEEGVATLRRSRRHHSQSPVRSEFKEEVSSGSRRAFGERSISVGKMPPKYALQPWLCPEFQQQAKEGDVLRKAWLSGHPSQTISKHKVEKTPLPPKCPSPFLTRSKVQEISSRLESVIAQESSKKPQRGRPPSKSFVNFMAQQVFSESAPSKVVLHPKPVARGRRRPSRSLLKPKLDDYAFLYNWDNEPKEDTTLQNLPMKQPFQLSRKPEEPQEVLPFSEGAPVKWNTSARGISQALGKLSVSVSLPNEWKSSEGQLPSTQPSQAFDVAKLQSPVLPVDSANVPVKWRISEGHQPPQSFFVDYQPQVSVDSASAAAKGASCWPMLKDSASTTNVKYSQGYKDFTKSTPTSVIKPATFTSAPAQKPMVSMGTYFKDEVPKCCDETGTSSLLPTSKADVENVFGVRLRSTSQKIGMKNPDPCKPFVLISAAARKEQANKGDLQGSVGGSKQSRPAFSFEKKQGNWPRYEGTLKKSEVFRPPGETSIWKANSPVEPAWITTLKQRQRGFVSQFFKKHRTRSEISVVTKDSRYEESKHITDIPPKKTFLSSFPGCAVPHLRHFPKSVAFYDQKMLHPRTRERLTRRSLSLPPTLPQREEPEWFSVAKKKAQAWSQIANIMQ

SEQ ID NO: 15 - DNA, 4318 bp, Mouse
gcagcctacaactacagaagcagaagttactactgtccagaagatgccatcagataaaggggatgtggagagggaattggccgacatagatgttgaagctcaaaaagaaccagcaccacaacctacatctacagatgtggcagagtccatgattagtggtccatcaccgcactgtgaagacaagaagaaaaaggataaagttggtgtgttgccatggatcaaaaaatctacaagccaaaaagaggtcactgtcttgaggaaagaagaggtccaagtgcatgtgcatccttctcgagtctgtggagaaggagaggaagcatcatgtcctgaattgcaaagggttcaaccccaaatgaaggtatcattggagagcacaacttaccacaaagagaagcatccaagaagtggtctcccggtcccttctacaagcatttcaagtgctactgcagaagatgatgtttctccaaagatgaatctgcctctccctctcagaaggcgaccaaatacaggggaaacctcctcagattcgaagagtacctcagaatatgagagtagttctgaaatgcaactgagtcctgcacattctttcaaaccgacaagaaagcctaaagatgatgctggtgctggaactgctgatgatgaagaagatggagatgatgagaaagaagagaaagacaatgatgatgatgatgaagaaaatgtcttcctcaaatcagaaaatgtagatgtagaagtgagcaagatggagaagcagcaggctctcagatattcttctaagttactaggaaaacccaaagctagagaggcctctgcaaaactagagagttcttcagaagatgagagaagttgtgaggaaatgacatctgcccattcttcccagtccttggaagagtttgaagaatgctcagaatcaagaggtttcatcagaggttccatctctgaagagcggccagcaaccaggtgccattcccaggccttgcaggagttggaggaaaaggaagtctccacagaatcaagtagttacattgagaagtacgagagttctgaggatctgagcagctcagagcaagagcagcaagtgcctccagtttcaaagagttctctaaaacagtggagtgctcctgccaagccagtgcaccccatacaaactgctcagcaacaacctcccgccatggtgaacatttctggaggaccaaagaaatctggagagccactgtctcccacccagactgtcaagtcacggctgagatctcagtctgagtgtcaagttcttgcagagccagaaggtgttgttgctgctgactgggacattttaatgcagcccccacccaggccaagacttgagcaagaagtttctgcaggtccagaacttactgtttttgaaaagagtattgctgtacagccttctggatatcctcctcagccttcagtgaaaccaagcatcaagaaagaaatatcgttgggtgcagaaagtgctcccttggagtccccacctcctcagcatcattttcagccactattgaagcccttcgtaaagcaacaggtatctgcttttgaaagagttatttctatggatccaaagcttccaacacagttccagccacggatgaagcctcaaggaaagcaatcattttcttccactccagagagcacagcatttgaagggagcacttctgtggaccatatgactctgaccctttcccagcctatgctacagccttatcaagaagtgcctttagaatcagagactgttgctgctaaaatgatttccacgggccaactacattccaaatattctgctcaccccttatcaagtcatcaaagccaaccagtcccagaaagcacctctgcagaaggagaggagctgctgccttctcagccttcagtgacacccaaattccagccactgatgacccaaggcagtattccttctgcatgggccattcctatttattcaccagctcccagaatggattctaagcccttgatgggatcagccatcgctttagagtcaaaaccttctaggtactctttgcagccatggcagagcacgccatttgaacaagtgtctgtaaccccagatcatgatcctgctgctgctgccgcttcatggagccctcctattgatccaccaacttccagaataccttctcagcccttgatgagattagctgtcaagcaaccaacctgtacagagttagcaagtgtttctgtgtcacagagcagctctctggaacgactgtcttccagattccccttccagccaagagcagatccagagcactatggagcagaggaaggagttgctacattgaggagatctagaaggcatcattctcagtccccagtgagatcagaattcaaggaagaagtttcctcaggttctcgaagagcttttggagaacggagcatttctgtgggaaaaatgcctcccaaatatgctcttcagccatggttgtgccccgaatttcagcagcaagccaaagagggggatgttttgaggaaggcctggctttcgggacatccttcccaaaccattagtaaacataaagtcgagaaaacaccactgcctcccaaatgtcccagcccattcttaaccagatctaaagttcaggaaatatcttcacgtctagagagtgttattgctcaagagagctccaagaaaccacagagaggcaggccaccttccaagtcctttgtgaacttcatggcacagcaagtattttcagagagtgctcctagtaaagtggtccttcatccaaagcctgttgctcggggccgaagacgtccttccagatccctgttgaagcctaaacttgacgattatgctttcttatataattgggataatgaacccaaggaagataccacattgcagaatctgcccatgaagcagccttttcagttatccagaaagcctgaagagccccaagaagttctgccattttcggagggtgcccctgtgaagtggaacacttctgccaggggtatctctcaggccctggggaagctctcagtttctgttagtcttccgaatgagtggaagagttctgaaggacagctgccttccacacagccgtctcaagcctttgatgtagctaagttgcagtcaccggttttaccagtggactcagcaaatgttcctgtgaagtggagaatttctgagggccatcagcctcctcagtctttctttgttgactatcagccacaagtttctgtagactctgccagtgctgctgctaaaggagccagctgctggcccatgctgaaagactcagcttcaacaacaaatgtcaaatacagtcaaggctacaaggatttcactaaaagcaccccaacttctgttatcaaacctgccacgttcacttctgctcctgcccagaaaccaatggtttccatgggcacttattttaaggatgaggttcccaagtgttgtgatgaaactggaacttcttcactcctacccaccagcaaggctgatgtcgaaaatgtttttggagtgcggttgcgaagtacctcacagaagattggaatgaagaatccggatccttgcaagccatttgtcctcatttcagcagcagcaaggaaagaacaagcaaacaaaggagatctccagggctcagttggaggctcaaagcagtccagaccagcatttagctttgagaagaagcaagggaattggcccagatatgaaggcactttgaagaagtcagaagttttcaggcctccaggagagacctctatttggaaggcaaattcccctgtggagccagcttggatcaccacgttaaaacagcggcaaaggggctttgtatcccaattttttaaaaaacacaggaccagatctgaaattagtgtggtgaccaaggattctagatatgaggaatctaaacacataactgatatcccaccaaagaagacttttctttccagtttccccggatgtgcggtgccacatttaaggcactttccaaaatcagttgcattttatgatcagaagatgcttcacccacgtaccagggaaagactaaccagacgatctttgagtcttccacccacacttccacaacgtgaggagcctgagtggttttcagtggccaagaagaaagcccaagcatggagccaaatagcaaatatcatgcaataaatagctcctagatggagcatcagcattttcagtaataattgcctgtaactatggtaattccatataatattttttgccagggctagtttttattagggaaaataactttagaaataggaattaacatttttaattattcaaatgaaataaatgctattaacatctaaccct

SEQ ID NO: 16 - DNA, 3853 bp, Mouse
gggaaagtagaggaggagagagcaacctggaggagaggttgacagcagggacacagacgacacttacgagcatacatctttgaactatgcaagccactgcagcgttagaaacagattctgataaaaattaccccaaaaatggaggacacttccaaaatgataaactttataatcctaagaaagaaaatatgttcttttccaatggttgcaacggagttattttggccttccctgatggtaaagaggactccttggcaacagaagagcgagctagtgacaaagagaacagcattgttgaccagagagacttatcagaactatctttttcagaaaatcaagattctaacagagggaatattttctcccagtcaagtgagtttgaagacagtaatgactatgcttttttgaatgagacctattctatccattattcagaatcaaaactcaaggatgaaaatcttttgcatttatattcaggattacatcctgaagtgcacaaaagagtagagatgatttttgacactttagataataatagcattggattgggaaggagcgccgaggcttcaggtgctgactgtggagacgtgcagaagagtgacgtggacgaagactcgcagcaggagtaccacagcgcagagctggagtgcataagtgcacacttagcgaagacagtaagcaggtccagtctagatgttagtgagttgaaaacatcaagctatgactttaaatgtggtggcaatttcgaagataatcatggtaaattagagagtggtcctagcccctctttggagtcccttaatggttttgcacaagaatgttcactccaagtctctacatctcaaagttctgatatgttacaagaatatcatgagccaaaatatgaaaaatgtaaggaacaagaggttgatttaacatatcataaagcatttgatggcattttacagagaagcagctctcccttaaaccatcagaaagtacctgaaactcaagtgtatactaaagaagtgaaatctcaaacaactgaaagtaaagacttttatggaaatagaattttccagaacaaagcattacagcgccctgagaatgctaccatgtttccccaagaccgagcccttgagacacacctgaaggctaatgatgctcaccagccgtctgggccttgtgctctggatgactcggtcatttctctctgtggctcttcacaatacaaaagcctccctgagccgggctttttctctcctgtgataccaagggtggcagtcacagattaccaggcagaagtagagggaagctgcctacaccacgtacaaggcagtgccacaaataaggcatgctcgctgatgaaagaagtgtgtcttacgtcagtgccagatgcagcagcatgtatagctgcagttcagcagacactgcatgtgagcagcagagtgaatgcaagctcttccatagtgtctgcatcaagcatcacagagacaaagatggtgaggcagagccaagctgaggagtggcaaagtgacaaacgaagcgtggcctgtaacaccgcctggtcgtgtgggcagcagtgcagagacgcacagagagctgccccaggaagtgactcaggaaggcctctctcaactggctgcttaaagcctagtgggaactccctgaatgagaattctttggagttaagaaaagtatttgataccacagacagacagaaacactgtaacagggcatttcagctctgtgaagagaaggctgtgccatccaggtgctgtcagaagaccactgagagggctataaaggcagagatgcaccttctggatgtttgctaccagatgtgccatcgacactgtcaccacatttacaaacttgtaatggaaagtagagcgggactcaataggaatttacaaactgattctgctaagaaggagttaggcgcagcactgctgtctgttttggaagacttgaaacttagatacatgaacttgaaaggaaaagtccataagggcatacctctggaagagctgcctccactgtcggtggagtccaaactcttatctgccttctccgattttgtttccaggctaatgaaagacgaagcatgcagcctttcaggagcaaattctgagctggataatcaaagcctacctgatgttgatgtttctccaggcctactaaaaacattgtcccagatgtcttttatacctgacagtagtcagcctgaacaaggtaaatcacccatgagcgatgtttgtaaaaatggtgatacagacataggctttaattgcctgaagctcaatgacaaagagtgtaaaaccgtccaggaagcaagtgaagactggtttgatgccacagagaggctgattggagctgacttctcagaaactcaggacagcacagcggaatgtgaggaatggcagccaaggaatccattagaattgaagaatagtgaactgcatggaaaaggtcagggctttttgatacacgtcggtggtctttgcccttctgtgtctgaggctgatttaaggtctcatttccagaaatatcaagtttctgaaatttcaatttatgattctactaattacagatatgcgtctcttgcttttgcaaaaaacagtaatgcgaagatggctgtgaaggaaatgaatggagtcaaaatcaatggaaaatctgtgaccgtgcggcttgtgaaaatccctggagagtacacaccaccacctctttccacaactgggaatagcactagtatgaatcatttggagaaaaataccaacaaagatgccacctcagcctcttctatttgtagactgcccagagctaagtcaaggcagctggaatctgagcaagacagtgagtttcctcccttggaccagggtgtcaagaaaaattgtaaccagatgaaatctggtcagttattacctgagacaccttttcagttcatacccccaaatacattgaaccttcgtagtttcaccaagatcatgaagagactggctgaactgcacccagacatcagcagagatcacattatagaggctcttcaggaagtgaggataaatcataaaggttttctgaacggtttgtccatcaataccattgtaaaaatggcttcatcttttctgagaaactctgctttgaaataaatgaagcaatacagagaaagtctttggaaagtatgaggacccttcatcagaggaagttctacagcaagtttggaaaaagctgagggtaatacaatcagacagctttgtaattggacctaacccgtttaagtattctgatagtcctttaaagggtggtgacaacagatagatagtaagatctgtaggatggttttaagtttataaacttcatttcaaaattattactcaagactccaagttgtattttgatttaaaaaaatgtggtcatgtccatttataaatcatcattaataagtccatttgaaaaatattgtccagaaaaagtttatggcccctatttcaaagttcattacacatgtaaaaagctatgtgaaatctttacacaattcttatgtgaaaaatgggatttctatgtagctagtttcagcttttcagacatagattggagtacggggttcagataagctgagtgtagtgagcagagaggtgctgcagtgtgagaggtcaccatgttccggcctggggtttgctatgttctgttgtactagagtacaagctgaatccctttctaatagtaactaacacaccgcttaaagctgcatttgtacttttcagatgtttacattgtttctacccagtgcttcattgttgtttcttgattaagattacccattaagcatttctatttgatgcctttgtgtttaataaaagtggttagctcctct

SEQ ID NO: 17 - protein, 1013 aa, Mouse
MQATAALETDSDKNYPKNGGHFQNDKLYNPKKENMFFSNGCNGVILAFPDGKEDSLATEERASDKENSIVDQRDLSELSFSENQDSNRGNIFSQSSEFEDSNDYAFLNETYSIHYSESKLKDENLLHLYSGLHPEVHKRVEMIFDTLDNNSIGLGRSAEASGADCGDVQKSDVDEDSQQEYHSAELECISAHLAKTVSRSSLDVSELKTSSYDFKCGGNFEDNHGKLESGPSPSLESLNGFAQECSLQVSTSQSSDMLQEYHEPKYEKCKEQEVDLTYHKAFDGILQRSSSPLNHQKVPETQVYTKEVKSQTTESKDFYGNRIFQNKALQRPENATMFPQDRALETHLKANDAHQPSGPCALDDSVISLCGSSQYKSLPEPGFFSPVIPRVAVTDYQAEVEGSCLHHVQGSATNKACSLMKEVCLTSVPDAAACIAAVQQTLHVSSRVNASSSIVSASSITETKMVRQSQAEEWQSDKRSVACNTAWSCGQQCRDAQRAAPGSDSGRPLSTGCLKPSGNSLNENSLELRKVFDTTDRQKHCNRAFQLCEEKAVPSRCCQKTTERAIKAEMHLLDVCYQMCHRHCHHIYKLVMESRAGLNRNLQTDSAKKELGAALLSVLEDLKLRYMNLKGKVHKGIPLEELPPLSVESKLLSAFSDFVSRLMKDEACSLSGANSELDNQSLPDVDVSPGLLKTLSQMSFIPDSSQPEQGKSPMSDVCKNGDTDIGFNCLKLNDKECKTVQEASEDWFDATERLIGADFSETQDSTAECEEWQPRNPLELKNSELHGKGQGFLIHVGGLCPSVSEADLRSHFQKYQVSEISIYDSTNYRYASLAFAKNSNAKMAVKEMNGVKINGKSVTVRLVKIPGEYTPPPLSTTGNSTSMNHLEKNTNKDATSASSICRLPRAKSRQLESEQDSEFPPLDQGVKKNCNQMKSGQLLPETPFQFIPPNTLNLRSFTKIMKRLAELHPDISRDHIIEALQEVRINHKGFLNGLSINTIVKMASSFLRNSALK

SEQ ID NO: 18 - protein, 1013 aa, Human; identical to SEQ ID NO: 17

SEQ ID NO: 19 - protein, 1047 aa, Human
MMQATAVVETASGKGYHSNGGNLQKDKPSNPKKENLLLSSNGCDEVKLTFPDDDWNSSTLEQRANNKEISNIDKMDLLEPFFSVSQDTNTESTQFQSSELEDSTDYAFLNKTYSIPYSESKLKKESLTPLSSELDPEVQKKEEVFFNILEHQDKTVGLERIYNISDANYRESAEDTQKHDTDEDSQQEYHSAEEQEYISNHLSFDQTKALDISNPEVVELGNSGYEVKCASNVEDNRVNSGSGSIISFDSLDVYGQEESLHVSKFQNSVMLREYHDLKHEKYKEQETNSMYHTVFDGSVLRSNSPGNQESQSKSGSLSPQKVLKMKIYTENMKSQINEGKDFCGNKIVENKILLHLENPSTLPQDKALETLLQPCKDCQTSWTSVFDDSIISACGYYESLQNTADSALDFSAMLPKIAVRDNQAIEDNTSLKVAHSSTTKKTCFHNIGEMCTKSLTDAASCTVTINQTVDVSTDFRACFTTSRATSARPSVVSTSSNTEITMMNKKRPDEWQNEKQKSVACSTDWSYSEDCIDTQMAITKGSGKSLSVDSLKPNGNFLNKDFLELRKACGITDLKKHPEREFQLFKDTEKDLPSMCCQKIMQRAIKAELHLLNVHYQMCRRHCCDIYKLVMENRNLSSNSAKKELGSALLSLLGDLKVRYVTLKEKIHKGIPLEELPPLSLESKLLSTFSTFASRLMKKETHVFSEADAEQDNQRAHDVDVSSNLKKTLSQMSLSSDNSHATQNISPKKDDFKNGDINADFSQLKLGDKDCRHYQETSEDWSDAKESLTGVDVSGTQGNQVEQDTWNLDLTGEMKNVEPSQRDKGYLIHVGGLCPSVSEADLRSHFQKYQVSEISIYDSTNYRYASLAFTKNSDAKIAVKEMNGIEINGKSVNVWPVKILGEYTSPLSSKNGNRISSNNLEKSTNKQIHSEFSISRLPRTRPRQLGSEQDSEVFPSDQGVKKNCKQIESAKLLPDTPVQFIPPNTLNLRSFTKIIKRLAELHPEVSRDHIINALQEVRIRHKGFLNGLSITTIVEMTSSLLKNSASS

(19) United States
(12) Patent Application Publication
(10) Pub. No.: US 2006/0223099 A1
(43) Pub. Date: Oct. 5, 2006

(54) TEX14 compositions and modulators thereof to alter intercellular bridge development and function

(75) Inventors: Martin M. Matzuk, Houston, TX (US); Michael P. Greenbaum, Houston, TX (US); Aleksandar Rajkovic, Houston, TX (US)

Correspondence Address: FULBRIGHT & JAWORSKI, LLP, 1301 MCKINNEY, SUITE 5100, HOUSTON, TX, 77010-3095, US

(73) Assignee: BAYLOR COLLEGE OF MEDICINE, Houston, TX (US)

(21) Appl. No.: 11/393,642

(22) Filed: Mar. 30, 2006

Related U.S. Application Data
(60) Provisional application No. 60/666,394, filed on Mar. 30, 2005.

(51) Int. Cl. C12Q 1/68

(52) U.S. Cl. 435/6

(57) ABSTRACT

The present invention is related to TEX14 and germ cell intercellular bridges and compositions thereof that can be used to inhibit and/or decrease the activity and/or expression of TEX14 and/or disrupt the formation, function, and/or integrity of the intercellular bridges, including localization of key components to the intercellular bridge, for example. In particular aspects, the methods and compositions are employed for fertility and infertility applications.

### STATEMENT REGARDING FEDERALLY SPONSORED RESEARCH OR DEVELOPMENT

This invention was made with government support under NICHD Grant Nos. HD07495, HD12629, HD42454, HD44858, and HD47514 awarded by the National Institutes of Health. The United States Government may have certain rights in the invention.

This invention claims priority to U.S. Provisional Patent Application Ser. No. 60,666,394, filed Mar. 30, 2005, which is incorporated by reference herein in its entirety.

### TECHNICAL FIELD

The present invention relates to at least the fields of fertility and medicine. In particular embodiments, it relates to pharmaceutical compositions and methods for modulating conception in animals.

### BACKGROUND OF THE INVENTION

Infertility is of great clinical significance, and between 2 and 7% of couples are infertile. Both physical and genetic factors are associated with male infertility. Some genetic factors are chromosomal aberrations, including at least the following: chromosomal translocations, Down's syndrome, Klinefilter's syndrome and Y chromosome microdeletions, for example. Many cases of azoospermia are idiopathic (have no obvious cause) in that the subject is infertile but otherwise healthy. Previous research has suggested that genetic factors are important contributors to these cases, but these factors have not been identified.

Intercellular bridges connect germ cells through large, cytoplasmic channels and are evolutionarily conserved from invertebrates to humans. While several genes important for ring canal function have been identified in Drosophila melanogaster (Hime et al., 1996; Hudson et al., 2002; Lu et al., 2004; Sokol et al., 1999; Brill et al., 2000), essential components of the mammalian intercellular bridge have until now not been described. Proposed roles for the intercellular bridge include germ cell communication, chromosome dosage compensation, and synchronization (Dym et al., 1971), and proteins and organelles have been observed to traverse the structure (Ventela et al., 2003).

The present invention provides novel compositions and methods for addressing fertility and infertility, particularly concerning a novel composition that in specific embodiments is associated with germ cell intercellular bridges.

### BRIEF SUMMARY OF THE INVENTION

The present invention is drawn to TEX14. TEX14 is a novel protein that localizes to germ cell intercellular bridges during spermatogenesis. Tex14 null mice are infertile due to a block in the early meiotic phase of spermatogenesis. Intercellular bridge densities, readily observed in spermatocyte intercellular bridges, were not seen by electron microscopy in Tex14 null mice, and heat shock factor 2 (HSF2) failed to localize to the intercellular bridge. Thus, TEX14 is the first mammalian protein that is essential for intercellular bridge development indicating that mammalian intercellular bridges are required for early male meiosis.

According to the present invention, the composition comprises a compound that disrupts intercellular bridges that interconnect germ cells admixed with a pharmaceutical carrier. The compound decreases the expression and/or activity of Tex14. Yet further, the compound also can alter the maintenance and function of the intercellular bridges.

In certain embodiments, the composition disrupts association of Tex14 with a binding partner. The binding partner comprises TEX14, TIP1, TIP2, TIP3, TIP4, HSF2, actin, delta tubulin, plectin, spermatogenic cell/sperm-associated keratin of molecular mass 57 kDa (SAK 57), or protocadherin alpha 3, in specific embodiments.

Yet further, it is envisioned that the composition of the present invention can be used as a male contraceptive.

In further embodiments, the present invention comprises a method of modulating fertility in a male subject comprising the step of administering the compound such that the compound disrupts intercellular bridges. The disruption of intercellular bridges results in a decrease in spermatogenesis and/or altered spermatogenesis. The disruption of intercellular bridges results in apoptosis of spermatocytes and/or germ cell.

Further embodiments comprise a method for identifying a substance that disrupts germ cell and/or spermatocyte intercellular bridge formation comprising the steps of: administering the test substance to a mammalian subject and determining the effects of the substance on germ cell and/or spermatocyte intercellular bridge formation, maintenance, or function, such that the substance reduces the production and/or function of sperm in said subject when compared to a control substance. Once the substance is identified, then it can be used for disrupting intercellular bridge formation in germ cells and/or spermatocytes in a subject by administering the substance to the subject.

Another embodiment of the present invention is a method of making or manufacturing an TEX14 inhibitor comprising: providing a candidate substance suspected of decreasing TEX14 expression or activity; selecting the TEX14 inhibitor by assessing the ability of the candidate substance to decrease TEX14 expression or activity; and making the selected TEX14 inhibitor. It is envisioned that the candidate substance may be a protein, a nucleic acid molecule, an organo-pharmaceutical, or a combination thereof. One of skill in the art is aware that once the inhibitor is identified, then it can be administered to a subject to alter intercellular bridge formation, function and/or maintence in a germ cell and/or spermatocyte resulting in contraception.

Another embodiment of the present invention is a method of making or manufacturing a compound that inhibits intercellular bridge formation, function and/or maintenance comprising: providing a candidate substance suspected of inhibiting intercellular bridge formation, function and/or maintenance; selecting the compound by assessing the ability of the candidate substance to inhibit intercellular bridge formation, function and/or maintenance; and making the selected compound. It is envisioned that the candidate substance may be an inhibitor of TEX14.

In certain embodiments, a method of enriching intercellular bridges may be employed, such as isolating or collecting a mitochondrial fraction from a tissue and/or other biological sample (e.g., testis), incubating the mitochondrial fraction with a detergent to disrupt the mitochondrial membranes and any other organelle membranes and collecting the intercellular bridge fraction.

In another embodiment of the invention, there is a method of identifying an inhibitor of TEX14, comprising the steps of: providing part or all of a TEX14 polypeptide, wherein said part or all of the TEX14 polypeptide comprises at least one TEX14 binding partner domain; providing a TEX14 binding partner; providing a test compound; and assaying for absence of binding of TEX14 to its binding partner in the presence of the test compound. In a specific aspect of the invention, the method further comprises the step of manufacturing the TEX14 inhibitor. In particular embodiments of the invention, the TEX 14 binding partner is TEX14, MKLP1, or MgcRacGAP. In specific embodiments, the assaying step comprises yeast two hybrid. In certain embodiments of a method of the invention, the inhibitor is further defined as a compound that inhibits germ cell intercellular bridge function and/or maintenance. The methods of the invention may further comprise delivering a TEX14 inhibitor to an individual, such as one in need of treatment for infertility or one in need of contraception.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIGS. 1A-1D Targeting of the Tex14 allele and creation of Tex14 mutant mice. FIG. 1A shows a targeting vector was constructed to replace exon 11 of the mouse Tex14 gene with a PgkHPRT expression cassette. The MC1tk expression cassette was used for negative selection. Twenty-two out of 70 (31.4%) of the ES cell clones analyzed were correctly targeted at the Tex14 locus. Four targeted ES cell clones were injected into blastocysts to produce 23 chimeric male mice (Matzuk et al., 1992), which were bred to produce F1 Tex14^{±} offspring. FIG. 1B shows a Southern blot analysis of genomic DNA derived from a litter from Tex^{±} (±) intercrosses. Male and female Tex14 homozygous null (−/−) mice were generated. WT, wild-type. FIG. 1C shows a Northern blot analysis of RNA derived from wild-type and Tex14 mutant mice. RNA was probed with 5′ Tex14 or Gapd cDNAs. FIG. 1D shows a Western blot analysis of testis samples from 11 day-old Tex14 WT, ±, and −/− mice. TEX14 was detected using a guinea pig polyclonal antibody raised to amino acids 885-1301 of mouse TEX14. The blot was reprobed with an antibody to β-actin as a control for sample loading.

FIGS. 2A-2I show gross and histological analysis of postnatal testes. (FIG. 1A) Gross analysis of adult testes from 7 week-old littermate mice. (FIGS. 2B-2I), Histological analysis of testes of 49 day-old Tex14± (FIGS. 2B, 2D) and Tex14^{-/-} (FIGS. 2C, 2E) mice, 10 day-old Tex14± (FIGS. 2F) and Tex14^{-/-} (FIG. 2G) mice, 14 day-old Tex14^{±} (FIG. 2H) and Tex14^{-/-} (FIG. 2I) mice, and 21 day-old Tex14^{±} (FIG. 2J) and Tex14^{-/-} (FIG. 2K) mice. Abbreviations are as follows: D, dying spermatocytes; L, leptotene spermatocyte; P, pachytene spermatocyte; PL, preleptotene spermatocyte; Sc, Sertoli cells; Sd6, stage 6 spermatids; Sd15, stage 15 spermatids; Sg-a, type A spermatogonium; Sg-b, type B spermatogonium, V, vacuoles. VI in panel (FIG. 2D) designates a stage VI seminiferous tubule. Scale bars are 200 um (FIG. 2B), 100 um (FIG. 2C), 15 um (FIGS. 2D, 2E), 20 um (FIGS. 2F-2I), and 40 um (FIGS. 2J, 2K).

FIGS. 3A-3F show quantitative and qualitative analysis of control and Tex14± testes. FIG. 3A shows a Northern blot analysis of 3 week-old and 8 week-old Tex14^{±} and Tex14^{-/-} littermates. FIG. 3B shows an immunohistochemical analysis of 21 day-old Tex]4^{±} and Tex14^{-/-} testes using antibodies to cyclin A1 (CCNA1) (arrows point to rare pachytene spermatocytes) or synaptonemal complex protein 3 (SCP3). FIGS. 3C and 3D show BrdU labeling (FIG. 3C) and quantitative analysis (FIG. 3D) of Brdu-labeled spermatogonia (Sg) and spermatocytes (Sp) in Tex14^{±} and Tex14⁻ testes. FIG. 3E shows DNA laddering analysis of testes from 10 day-old, 14 day-old, and 56 day-old mice. FIG. 3F shows TUNEL analysis was performed on the testes of 10 day-old and 14 day-old Tex14^{±}and Tex14⁻ littermates as shown.

FIGS. 4A-4G show immunohistological localization of TEX14 to intercellular bridges. FIG. 4A shows immunofluorescence of a 6 week-old wild-type seminiferous tubule in which staining for TEX14 (red), actin (green), and DNA (DAPI, blue) were merged. Note the presence of the TEX14-positive (red) rings throughout the tubule. FIGS. 4B-4D show immunofluorescence of 9 day-old wild-type intercellular bridges showing TEX14 (FIG. 4B), HSF2 (FIG. 4C), and merged (FIG. 4D). FIG. 4E shows immunohistochemistry labeling of TEX14 at intercellular bridges between spermatocytes (black arrows) and spermatogonia (black arrowhead). FIGS. 4F-4G show HSF2 immunofluorescence of 9 day-old Tex14 null testis lacking intercellular bridge localization (FIG. 4F) and the corresponding HSF2 protein levels in 9 day-old testis by Western blot analysis (FIG. 4G).

FIGS. 5A-5B show histologic analysis of 10 day-old wild-type (FIG. 5A) and Tex14^{-/-} (FIG. 5B) testes.

FIGS. 6A-6F show immunohistochemical analysis of testes from control and Tex14^{-/-} testes. FIGS. 6A and 6B show Tex14^{±} (FIG. 6A) and Tex14^{-/-} (FIG. 6B) 5 day testis incubated with anti-TEX14 immune serum. FIGS. 6C and 6D show Tex14^{±} (FIG. 6C) and Tex14^{-/-} (FIG. 6D) 7 day testis incubated with anti-TEX14 immune serum. FIG. 6D shows Tex14^{-/- 7} day testes incubated with an anti-TEX14 antibody. FIGS. 6E and 6F show wild-type 21 day testes incubated with guinea pig anti-TEX14 immune serum (FIG. 6E) and the corresponding pre-immune serum (FIG. 6F).

FIG. 7A and FIG. 7B show phosphorylation of a poly-tyrosine containing substrate (FIG. 7A) by the addition of Src kinase, TEX14, or a negative control, or phosphorylation of myelin basic protein (FIG. 7B) by the addition of MAPK2, TEX14, or a negative control. Phosphorylated tyrosine or serine/threonine amino acids were detected with HRP conjugated antibodies catalyzing a Tetramethylbenzidine (TMB) reaction. Activity was measured by OD₄₅₀.

FIGS. 8A-8D demonstrate immunolocalization of TEX14 and PLZF in wild-type and Tex14^{-/-} mice. TEX14 (red) localizes to the intercellular bridges between PLZF positive spermatogonia (green) in 8 day-old, neonatal (FIG. 8A) and 8 week-old, mature (FIG. 8B) mice. PLZF labeling of whole mount seminiferous tubules shows Aₛ, Aₚₜ) and Aₐₗ spermatogonia in 35 day-old Tex14^{±} tubules (FIG. 8C), whereas predominantly Aₛ spermatogonia appear to be present in 35 day-old Tex14^{-/-} tubules (FIG. 8D).

FIGS. 9A-9F show histological analysis of 10 day-old Tex14^{±} (FIG. 9A) and Tex14^{-/-} (FIG. 9B) testes, 3 month-old Tex14^{±} (FIG. 9C) and Tex14^{-/-} (FIG. 9D) testes, and 1 year-old Tex14−/− testes at low (FIG. 9E) and high (FIG. 9F) magnification. Arrows point to dying spermatocytes Sg-a, type A spermatogonium Sg-b, type B spermatogonium. Scale bars are 100 um (FIGS. 9A-9E) and 25 um (FIG. 9F).

FIGS. 10A-10D show low magnification view (5×) of CCNA1 immunohistochemistry demonstrating many positive cells in 21 day-old Tex14^{±} (FIG. 10A). CCNA1 positive cells (arrows) are rarely found in Tex14^{-/-} (FIG. 10B) testes. Similar numbers of PLZF-positive cells (arrows) are seen in 21 day-old Tex14^{±} (FIG. 10C) and Tex14^{-/-} (FIG. 10D) testes. Scale bars are 200 um (FIGS. 10A, 10B) and 50 um (FIGS. 10C, 10D).

FIGS. 11A-11D show at least electron microscopy of 11 day-old control testis. Intercellular bridges connecting germ cells are marked with arrows in FIGS. 11A and 11B. In FIG. 11C, a higher magnification view of the inset (white box) in FIG. 11B clearly shows the electron dense intercellular bridge densities of one intercellular bridge. Thirty bridges were found in about 60 minutes of search time on control section (FIG. 11D). No bridges were found in six hours of search timeon Tex14^{-/-} sections. Abbreviations are as follows: ICBD, intercellular bridge density; M, mitochondrion.

FIG. 12 illustrates that MKLP1 and RacGAP1, which form the centralspindlin complex, colocalize with TEX14 in the intercellular bridge and are considered components of the mammalian intercellular bridge. MKLP1 (FIG. 12C) and TEX14 (FIG. 12B) colocalize in the intercellular bridges of 14 day-old mouse testis (FIG. 12A). RacGAP1 (FIG. 12F) and TEX14 (FIG. 12E) also colocalize in 14 day-old mouse intercellular bridges (FIG. 12D). Arrows in FIGS. 12D-12F indicate intercellular bridges.

FIG. 13 demonstrates co-localization of TEX14, MKLP1, and MgcRacGAP in human intercellular bridges.

FIG. 14 illustrates exemplary targeted yeast-two-hybrid interactions between TEX14 and exemplary centralspindlin proteins. In this particular type of assay, TEX14 interacts strongly with itself and MKLP1 but very weakly with MgcRacGAP by targeted yeast-two-hybrid analysis. In other embodiments, interaction between TEX14 and MgcRacGAP is not weak, as detected by other assays. Murine p53 interacts with and SV40 large T-antigen in the positive control, and human Lamin C fails SV40 large T-antigen in the negative control.

### DETAILED DESCRIPTION OF THE INVENTION

It is readily apparent to one skilled in the art that various embodiments and modifications can be made to the invention disclosed in this Application without departing from the scope and spirit of the invention.

### I. DEFINITIONS

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms. Some embodiments of the invention may consist of or consist essentially of one or more elements, method steps, and/or methods of the invention. It is contemplated that any method or composition described herein can be implemented with respect to any other method or composition described herein.

As used herein, the term “animal” refers to any animal, although in specific embodiments the animal is a mammal, such as human, non-human primates, horse, cow, cat, dog, rat or mouse. In specific embodiments, the animal is a human.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting. Thus, one of skill in the art understands that the term “antibody” refers to any antibody-like molecule that has an antigen binding region, and includes antibody fragments such as Fab′, Fab, F(ab′)2, single domain antibodies (DABs), Fv, scFv (single chain Fv), and the like. The techniques for preparing and using various antibody-based constructs and fragments are well known in the art. (See, e.g., Antibodies: A Laboratory Manual, Cold Spring Harbor Laboratory, 1988).

As used herein, the term “binding protein” refers to proteins that demonstrate binding affinity for a specific ligand. Binding proteins may be produced from separate and distinct genes. For a given ligand, the binding proteins that are produced from specific genes are distinct from the ligand binding domain of the receptor or its soluble receptor.

As used herein, the term “binding partner” or “interacting proteins” refer to at least one molecule capable of binding another molecule with specificity, as for example, an antigen and an antigen-specific antibody or an enzyme and its inhibitor. Exemplary binding partners may include, for example, biotin and avidin or streptavidin, IgG and protein A, receptor-ligand couples, protein-protein interaction, and complementary polynucleotide strands. In certain embodiments, the binding partner comprises TEX14, TIP1, TIP2, TIP3, TIP4, HSF2, actin, delta tubulin, plectin, spermatogenic cell/sperm-associated keratin of molecular mass 57 kDa (SAK 57) or protocadherin alpha 3, for example. The term “binding partner” may also refer to polypeptides, lipids, small molecules, or nucleic acids that bind to TEX14 in cells. A change in the interaction between a protein and a binding partner can manifest itself as an increased or decreased probability that the interaction forms, or an increased or decreased concentration of TEX14 in cells-binding partner complex.

As used herein, the term “TEX14 binding fragment” refers to the nucleic acid fragment and/or amino acid fragment of TEX14 respectively that is capable of binding to the binding partner or interacting protein, for example polypeptides, lipids, small molecules, or nucleic acids.

As used herein, the term “conception” refers to the union of the male sperm and the ovum of the female, which may also be referred to as fertilization.

As used herein, the term “contraception” refers to the prevention of conception. A contraceptive device, thus, refers to any process, device, or method that prevents conception, development of the pre-implantation embryo, and/or implantation. Well-known categories of contraceptives include, steroids, chemical barrier, physical barrier; combinations of chemical and physical barriers; abstinence and permanent surgical procedures. Contraceptives can be administered to either males or females.

As used herein, the term “DNA” is defined as deoxyribonucleic acid.

As used herein, the term “DNA segment” refers to a DNA molecule that has been isolated free of total genomic DNA of a particular species. Included within the term “DNA segment” are DNA segments and smaller fragments of such segments, and also recombinant vectors, including, for example, plasmids, cosmids, phage, viruses, and the like.

As used herein, the term “expression construct” or “transgene” is defined as any type of genetic construct containing a nucleic acid coding for gene products in which part or all of the nucleic acid encoding sequence is capable of being transcribed can be inserted into the vector. The transcript is translated into a protein, but it need not be. In certain embodiments, expression includes both transcription of a gene and translation of mRNA into a gene product. In other embodiments, expression only includes transcription of the nucleic acid encoding genes of interest. In the present invention, the term “therapeutic construct” may also be used to refer to the expression construct or transgene. One skilled in the art realizes that the present invention utilizes the expression construct or transgene as a therapy to treat infertility. Yet further, the present invention utilizes the expression construct or transgene as a “prophylactic construct” for contraception. Thus, the “prophylactic construct” is a contraceptive.

As used herein, the term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well and are described infra.

As used herein, the term “gene” is used for simplicity to refer to nucleic acid sequences, such as polynucleotides, that encode a functional protein, polypeptide or peptide. This functional term includes both genomic sequences, cDNA sequences and engineered segments that express, or may be adapted to express, proteins, polypeptides, domains, peptides, fusion proteins and mutant.

As used herein, the term “fertility” refers to the quality of being productive or able to conceive. Fertility relates to both male and female animals.

As used herein, the term “infertility” refers to the inability or diminished ability to conceive or produce offspring. Infertility can be present in either male or female. In the present invention, administration of a composition to enhance infertility or decrease fertility is reversible. In the present invention, administration of a composition to enhance infertility or decrease fertility is reversible. Examples of direct or indirect causes of infertility include, without limitation, azoospermia; genetic disorders associated with defective spermatogenesis (e.g., Klinefelter's syndrome and gonadal dysgenesis); oligospermia, varicocele, and other sperm disorders relating to low sperm counts, sperm motility, and sperm morphology; and ovulatory dysfunction (e.g., polycystic ovary syndrome (PCOS) or chronic anovulation).

As used herein, the term “inhibitor” refers to a compound or composition that decreases TEX14 activity and/or expression. For example, an inhibitor can decrease TEX14 gene or protein activity. An inhibitor can be a polynucleotide, a polypeptide, an antibody, or a small molecule.

As used herein, the term “modulate” refers to the suppression, enhancement, or induction of a function. For example, “modulation” or “regulation” of gene expression refers to a change in the activity of a gene. Modulation of expression can include, but is not limited to, gene activation and gene repression. “Modulate” or “regulate” also refers to methods, conditions, or agents which increase or decrease the biological activity of a protein, enzyme, inhibitor, signal transducer, receptor, transcription activator, co-factor, and the like. This change in activity can be an increase or decrease of mRNA translation, DNA transcription, and/or mRNA or protein degradation, which may in turn correspond to an increase or decrease in biological activity. Such enhancement or inhibition may be contingent upon occurrence of a specific event, such as activation of a signal transduction pathway and/or may be manifest only in particular cell types.

As used herein, the term “modulated activity” refers to any activity, condition, disease or phenotype that is modulated by a biologically active form of a protein. Modulation may be affected by affecting the concentration of biologically active protein, e.g., by regulating expression or degradation, or by direct agonistic or antagonistic effect as, for example, through inhibition, activation, binding, or release of substrate, modification either chemically or structurally, or by direct or indirect interaction which may involve additional factors.

As used herein, the term “modulator” refers to any composition and/or compound that alters the expression of a specific activity, such as TEX14 activity or expression. The modulator is intended to comprise any composition or compound, e.g., antibody, small molecule, peptide, oligopeptide, polypeptide, or protein.

The term “small molecule” refers to a synthetic or naturally occurring chemical compound, for instance a peptide or oligonucleotide that may optionally be derivatized, natural product or any other low molecular weight (typically less than about 5 kDalton) organic, bioinorganic or inorganic compound, of either natural or synthetic origin. Such small molecules may be a therapeutically deliverable substance or may be further derivatized to facilitate delivery.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic and/or prophylactic compositions is contemplated. Supplementary active ingredients also can be incorporated into the compositions.

As used herein, the term “polynucleotide” is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means. Furthermore, one skilled in the art is cognizant that polynucleotides include mutations of the polynucleotides, include but are not limited to, mutation of the nucleotides, or nucleosides by methods well known in the art.

As used herein, the term “polypeptide” is defined as a chain of amino acid residues, usually having a defined sequence. As used herein the term polypeptide is interchangeable with the terms “peptides” and “proteins”.

As used herein, the term “promoter” is defined as a DNA sequence recognized by the synthetic machinery of the cell, or introduced synthetic machinery, required to initiate the specific transcription of a gene.

As used herein, the term “purified protein or peptide”, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

As used herein, the term “RNA” is defined as ribonucleic acid.

As used herein, the term “RNA interference” or “RNAi” is an RNA molecule that is used to inhibit a particular gene of interest.

As used herein, the term “stimulator” is defined as a compound or composition that enhances the activity of TEX14. The enhanced activity can be TEX14 gene activity and/or TEX14 protein activity. A stimulator can be a polynucleotide, a polypeptide, an antibody, or a small molecule.

As used herein, the term “under transcriptional control” or “operatively linked” is defined as the promoter is in the correct location and orientation in relation to the nucleic acid to control RNA polymerase initiation and expression of the gene.

As used herein, one of skill in the field understands that “Tex14” denotes a mouse gene and “TEX14” denotes a human gene. However, the scope of the present invention covers any vertebrate TEX14 gene or protein and should not be limited to a mouse or human gene or protein. Thus, as used herein Tex14 and TEX14 or any other annotation of TEX14 is within the scope of the present invention and are interchangeable.

### II. TEX14 INHIBITORS

In certain embodiments, inhibitors of TEX14 are administered to a subject to reduce or inhibit the activity and/or expression of TEX14. It is envisioned that TEX14 plays a role in intercellular bridge development during spermatogenesis. It is also envisioned that TEX14 can play a role in intercellular maintenance and/or function. Thus, an inhibitor of TEX14 can decrease the formation of intercellular bridge, alter function, and/or alter maintenance of the intercellular bridges. Inhibition of TEX14 also leads to an increase in apoptosis in germ cells and a disruption in meiotic DNA sysnthesis, in specific embodiments.

The inhibitors of the present invention include, but are not limited to polynucleotides (RNA or DNA), polypeptides, antibodies, small molecules or other compositions that are capable of inhibiting either the activity and/or the expression of TEX14. Still further, other inhibitors of TEX14, include, but are not limited to compositions discussed in U.S. Application No. US 2002/0081592, which is incorporated herein by reference in its entirety.

In this patent, the term “TEX14 gene product” refers to proteins and polypeptides having amino acid sequences that are substantially identical to the native TEX14 amino acid sequences (or RNA, if applicable) or that are biologically active, in that they are capable of performing functional activities similar to an endogenous TEX14 and/or cross-reacting with anti-TEX14 antibody raised against TEX14.

The term “TEX14 gene product” also includes related compounds of the respective molecule that exhibit at least some biological activity in common with its native counterpart. Such related compounds include, but are not limited to, truncated polypeptides and polypeptides having fewer amino acids than the native polypeptide. The TEX14 polypeptide sequences include, but are not limited to SEQ.ID.NO.1 (GenBank Accession No. NP_{—}113563), SEQ.ID.NO.2 (GenBank Accession No. NP_{—}938207), SEQ.ID.NO.3 (GenBank Accession No. NP_{—}112562), SEQ.ID.NO.4 (GenBank Accession No. AAK31963), SEQ.ID.NO.5 (GenBank Accession No. DAA01357), and SEQ.ID.NO.6 (GenBank Accession No. DAA01358). Other TEX14 sequences that may be used in the present invention are more fully described in U.S. Application No. US 2002/0081592, which is incorporated herein by reference in its entirety.

The term “TEX14 gene” “TEX14 polynucleotide” or “TEX14 nucleic acid” refers to at least one molecule or strand of DNA (e.g., genomic DNA, cDNA) or RNA sequence (antisense RNA, siRNA) a derivative or mimic thereof, comprising at least one nucleotide base, such as, for example, a naturally occurring purine or pyrimidine base found in DNA (e.g., adenine “A,” guanine “G,” thymine “T,” and cytosine “C”) or RNA (e.g., A, G, uracil “U,” and C). The term “nucleic acid” encompasses the terms “oligonucleotide” and “polynucleotide.” These definitions generally refer to at least one single-stranded molecule, but in specific embodiments will also encompass at least one additional strand that is partially, substantially or fully complementary to the at least one single-stranded molecule. Thus, a nucleic acid may encompass at least one double-stranded molecule or at least one triple-stranded molecule that comprises one or more complementary strand(s) or “complement(s)” of a particular sequence comprising a strand of the molecule. An “isolated nucleic acid” as contemplated in the present invention may comprise transcribed nucleic acid(s), regulatory sequences, coding sequences, or the like, isolated substantially away from other such sequences, such as other naturally occurring nucleic acid molecules, regulatory sequences, polypeptide or peptide encoding sequences, etc.

More particularly, a “TEX14 gene or TEX14 polynucleotide” may also comprise any combination of associated control sequences. The TEX14 polynucleotide sequences include, but are not limited to SEQ.ID.NO:7 (GenBank Accession No. NM_{—}031386), SEQ.ID.NO:8 (GenBank Accession No. NM_{—}198393), SEQ.ID.NO:9 (GenBank Accession No. NM_{—}031272), SEQ.ID.NO:10 (GenBank Accession No. AF285584), SEQ.ID.NO:11 (GenBank Accession No. BK000966), SEQ.ID.NO:12 (GenBank Accession No. BK000967). Other TEX14 sequences that may be used in the present invention are more fully described in U.S. Application No. US 2002/0081592, which is incorporated herein by reference in its entirety. Thus, nucleic acid compositions encoding TEX14 are herein provided and are also available to a skilled artisan at accessible databases, including the National Center for Biotechnology Information's GenBank database and/or commercially available databases, such as from Celera Genomics, Inc. (Rockville, Md.). Also included are splice variants that encode different forms of the protein, if applicable. The nucleic acid sequences may be naturally occurring or synthetic.

Still further, the “TEX14 nucleic acid sequence,” “TEX14 polynucleotide,” and “TEX14 gene product” refer to nucleic acids provided herein, homologs therof, and sequences having substantial similarity and function, respectively. The term “substantially identical”, when used to define either a TEX14 amino acid sequence or TEX14 polynucleotide sequence, means that a particular subject sequence, for example, a mutant sequence, varies from the sequence of natural TEX14, respectively, by one or more substitutions, deletions, or additions, the net effect of which is to retain at least some of the biological activity found in the native TEX14 protein, respectively. Alternatively, DNA analog sequences are “substantially identical” to specific DNA sequences disclosed herein if: (a) the DNA analog sequence is derived from coding regions of the natural TEX14 gene, respectively; or (b) the DNA analog sequence is capable of hybridization to DNA sequences of TEX14 under moderately stringent conditions and TEX14, respectively having biological activity similar to the native proteins; or (c) DNA sequences that are degenerative as a result of the genetic code to the DNA analog sequences defined in (a) or (b). Substantially identical analog proteins will be greater than about 80% similar to the corresponding sequence of the native protein. Sequences having lesser degrees of similarity but comparable biological activity are considered to be equivalents. In determining polynucleotide sequences, all subject polynucleotide sequences capable of encoding substantially similar amino acid sequences are considered to be substantially similar to a reference polynucleotide sequence, regardless of differences in codon sequence.

As used herein, “hybridization”, “hybridizes” or “capable of hybridizing” is understood to mean the forming of a double or triple stranded molecule or a molecule with partial double or triple stranded nature. The term “hybridization”, “hybridize(s)” or “capable of hybridizing” encompasses the terms “stringent condition(s)” or “high stringency” and the terms “low stringency” or “low stringency condition(s)” or “moderately stringent conditions”.

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. For example, a medium or moderate stringency condition could be provided by about 0.1 to 0.25 M NaCl at temperatures of about 37° C. to about 55° C. Under these conditions, hybridization may occur even though the sequences of probe and target strand are not perfectly complementary, but are mismatched at one or more positions. In another example, a low stringency condition could be provided by about 0.15 M to about 0.9 M salt, at temperatures ranging from about 20° C. to about 55° C. Of course, it is within the skill of one in the art to further modify the low or high stringency conditions to suite a particular application. For example, in other embodiments, hybridization may be achieved under conditions of, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

Naturally, the present invention also encompasses nucleic acid sequences that are complementary, or essentially complementary, to the sequences set forth herein, for example, in SEQ ID NO:7, SEQ ID NO:8, SEQ ID NO:9, SEQ ID NO:10, SEQ ID NO:11, or SEQ ID NO:12. Nucleic acid sequences that are “complementary” are those that are capable of base-pairing according to the standard Watson-Crick complementarity rules. As used herein, the terms “complementary sequences” and “essentially complementary sequences” means nucleic acid sequences that are substantially complementary to, as may be assessed by the same nucleotide comparison set forth above, or are able to hybridize to a nucleic acid segment of one or more sequences set forth herein. Such sequences may encode an entire TEX14 molecule or functional or non-functional fragments thereof.

A. Expression Vectors

The present invention may involve using expression constructs as the pharmaceutical composition and/or diagnostic compositions. In certain embodiments, it is contemplated that the expression construct comprises polynucleotide sequences encoding polypeptides which can act as inhibitors of TEX14 and/or TEX14-related compounds or related compounds, for example the expression construct may comprise a nucleic acid sequence encoding an antisense molecule or an siRNA molecule. One of skill of the would be able to determine depending upon the desired usage of the expression construct whether the polynucleotide sequences should encode a polypeptide that functions as an inhibitor of TEX14 (therapeutic/prophylactic protocols) or a TEX 14-related compound (diagnostic protocols).

In certain embodiments, the present invention involves the manipulation of genetic material to produce expression constructs that encode inhibitors TEX14 and/or TEX14-related compounds. Thus, the TEX14 inhibitor and/or related compound is contained in an expression vector. Such methods involve the generation of expression constructs containing, for example, a heterologous nucleic acid sequence encoding an inhibitor of interest and a means for its expression, replicating the vector in an appropriate cell, obtaining viral particles produced therefrom, and infecting cells with the recombinant virus particles.

As used in the present invention, the term “expression vector” refers to any type of genetic construct comprising a nucleic acid coding for TEX14 inhibitor and/or related compounds. In some cases, DNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra. It is contemplated in the present invention, that virtually any type of vector may be employed in any known or later discovered method to deliver nucleic acids encoding an inhibitor of TEX14 or related molecules. Where incorporation into an expression vector is desired, the nucleic acid encoding an TEX14 inhibitor or related molecule may also comprise a natural intron or an intron derived from another gene. Such vectors may be viral or non-viral vectors as described herein, and as known to those skilled in the art. An expression vector comprising a nucleic acid encoding an TEX14 inhibitor or related molecule may comprise a virus or engineered construct derived from a viral genome.

In particular embodiments of the invention, a plasmid vector is contemplated for use to transform a host cell. In general, plasmid vectors containing replicon and control sequences that are derived from species compatible with the host cell are used in connection with these hosts. The vector ordinarily carries a replication site, as well as marking sequences that are capable of providing phenotypic selection in transformed cells. Plasmid vectors are well known and are commercially available. Such vectors include, but are not limited to, the commercially available pSupervector (OligoEngine, Seattle, Wash.), pSuppressorNeo vector (IMGENEX Corporation) and pSilencer™ siRNA expression vectors (Ambion, Austin Tex.). Other vectors that may be employed in the present invention include, but are not limited to, the following eukaryotic vectors: pWLNEO, pSV2CAT, pOG44, PXT1, pSG (Stratagene) pSVK3, pBSK, pBR322, pUC vectors, vectors that contain markers that can be selected in mammalian cells, such as pCDNA3.1, episomally replicating vectors, such as the pREP series of vectors, pBPV, pMSG, pSVL (Pharmacia), adenovirus vector (AAV; pCWRSV, Chatterjee et al. (1992)); retroviral vectors, such as the pBABE vector series, a retroviral vector derived from MoMuLV (pGlNa, Zhou et al., (1994)); and pTZ18U (BioRad, Hercules, Calif.).

In one embodiment, a gene encoding a TEX14 inhibitor or structural/functional domain thereof or a TEX14-related compound is introduced in vivo in a viral vector. The ability of certain viruses to enter cells via receptor-mediated endocytosis and to integrate into the host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). Such vectors include an attenuated or defective DNA virus, such as but not limited to herpes simplex virus (HSV), papilloma virus, Epstein Barr virus (EBV), adenovirus, adeno-associated virus (AAV), lentivirus and the like. Defective viruses, which entirely or almost entirely lack viral genes, are preferred. Defective virus is not infective after introduction into a cell. Use of defective viral vectors allows for administration to cells in a specific, localized area, without concern that the vector can infect other cells. Thus, any tissue can be specifically targeted. Examples of particular vectors include, but are not limited to, a defective herpes virus 1 (HSV1) vector (Kaplitt et al., 1991) an attenuated adenovirus vector, (Stratford-Perricaudet et al., 1992), and a defective adeno-associated virus vector (Samulski et al., 1987 and Samulski et al., 1989). Such vectors may be used to (i) transform cell lines in vitro for the purpose of expressing the TEX14 molecules or inhibitors thereof, such as antisense or siRNA molecules of the present invention or (ii) to transform cells in vitro or in vivo to provide therapeutic/prophylactic molecules for gene therapy. Thus, the present invention contemplates viral vectors such as, but not limited to, an adenoviral vector, an adeno-associated viral vector, a retroviral vector, a lentiviral vector, a herpes viral vector, polyoma viral vector or hepatitis B viral vector.

Preferably, for in vivo administration, an appropriate immunosuppressive treatment is employed in conjunction with the viral vector, e.g., adenovirus vector, to avoid immunodeactivation of the viral vector and transfected cells. For example, immunosuppressive cytokines, such as interleukin-12 (IL-12), interferon-y (IFN-γ), or anti-CD4 antibody, can be administered to block humoral or cellular immune responses to the viral vectors (Wilson, Nature Medicine (1995). In addition, it is advantageous to employ a viral vector that is engineered to express a minimal number of antigens.

In another embodiment the gene can be introduced in a retroviral vector, e.g., as described in U.S. Pat. No. 5,399,346; Mann et al., 1983; U.S. Pat. No. 4,650,764; U.S. Pat. No. 4,980,289; Markowitz et al., 1988; U.S. Pat. No. 5,124,263; International Patent Publication No. WO 95/07358; and Kuo et al., 1993, each of which is incorporated herein by reference in its entirety. Targeted gene delivery is described in International Patent Publication WO 95/28494.

Alternatively, the vector can be introduced in vivo by lipofection. For the past decade, there has been increasing use of liposomes for encapsulation and transfection of nucleic acids in vitro. Synthetic cationic lipids designed to limit the difficulties and dangers encountered with liposome mediated transfection can be used to prepare liposomes for in vivo transfection of a gene encoding a marker. The use of cationic lipids may promote encapsulation of negatively charged nucleic acids, and also promote fusion with negatively charged cell membranes. The use of lipofection to introduce exogenous genes into the specific organs in vivo has certain practical advantages. Molecular targeting of liposomes to specific cells represents one area of benefit. Lipids may be chemically coupled to other molecules for the purpose of targeting. Targeted peptides, e.g., hormones or neurotransmitters, and proteins such as antibodies, or non-peptide molecules could be coupled to liposomes chemically.

It is also possible to introduce the vector in vivo as a naked DNA plasmid. Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun, or use of a DNA vector transporter (Wu and Wu, 1988).

As used herein, the terms “cell,” “cell line,” and “cell culture” may be used interchangeably. All of these terms also include their progeny, which is any and all subsequent generations formed by cell division. It is understood that all progeny may not be identical due to deliberate or inadvertent mutations. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, TEX14 molecule or antisense or siRNA or a construct thereof. Therefore, recombinant cells are distinguishable from naturally occurring cells that do not contain a recombinantly introduced nucleic acid.

In certain embodiments, it is contemplated that nucleic acid or proteinaceous sequences may be co-expressed with other selected nucleic acid or proteinaceous sequences in the same host cell. Co-expression may be achieved by co-transfecting the host cell with two or more distinct recombinant vectors. Alternatively, a single recombinant vector may be constructed to include multiple distinct coding regions for nucleic acids, which could then be expressed in host cells transfected with the single vector.

A gene therapy vector as described above can employ a transcriptional control sequence operably associated with the sequence for the TEX14 inhibitor or related compound inserted in the vector. Such an expression vector is particularly useful to regulate expression of a therapeutic TEX14 inhibitor.

B. Transcription Factors and Nuclear Binding Sites

Transcription factors are regulatory proteins that binds to a specific DNA sequence (e.g., promoters and enhancers) and regulate transcription of an encoding DNA region. Typically, a transcription factor comprises a binding domain that binds to DNA (a DNA binding domain) and a regulatory domain that controls transcription. Where a regulatory domain activates transcription, that regulatory domain is designated an activation domain. Where that regulatory domain inhibits transcription, that regulatory domain is designated a repression domain.

Activation domains, and more recently repression domains, have been demonstrated to function as independent, modular components of transcription factors. Activation domains are not typified by a single consensus sequence but instead fall into several discrete classes: for example, acidic domains in GAL4 (Ma et al., 1987), GCN4 (Hope et al., 1987), VP16 (Sadowski et al., 1988), and GATA-1 (Martin, et al. 1990); glutamine-rich stretches in Sp1 (Courey et al., 1988) and Oct-2/OTF2 (Muller-Immergluck et al., 1990; Gerster et al., 1990); proline-rich sequences in CTF/NF-1 (Mermod et al., 1989); and serine/threonine-rich regions in Pit-1/GH-F-1 (Theill et al., 1989) all function to activate transcription. The activation domains of fos and jun are rich in both acidic and proline residues (Abate et al., 1991; Bohmann et al., 1989); for other activators, like the CCAAT/enhancer-binding protein C/EBP (Friedman et al., 1990), no evident sequence motif has emerged.

In certain embodiments, the transcription factor is a heat shock transcription factor, such as HSF2 which may also have a non-transcription factor fuction at the intercellular bridge.

C. Antisense and Ribozymes

An antisense molecule that binds to a translational or transcriptional start site, or splice junctions, are ideal inhibitors. Antisense, ribozyme, and double-stranded RNA molecules target a particular sequence to achieve a reduction or elimination of a particular polypeptide, such as TEX14. Thus, it is contemplated that antisense, ribozyme, and double-stranded RNA, and RNA interference molecules are constructed and used to inhibit TEX14.

1. Antisense Molecules

Antisense methodology takes advantage of the fact that nucleic acids tend to pair with complementary sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNAs, are employed to inhibit gene transcription or translation or both within a host cell, either in vitro or in vivo, such as within a host animal, including a human subject.

Antisense constructs are designed to bind to the promoter and other control regions, exons, introns or even exon-intron boundaries of a gene. It is contemplated that the most effective antisense constructs may include regions complementary to intron/exon splice junctions. Thus, antisense constructs with complementarity to regions within 50-200 bases of an intron-exon splice junction are used. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

It is advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

2. RNA Interference

It is also contemplated in the present invention that double-stranded RNA is used as an interference molecule, e.g., RNA interference (RNAi). RNA interference is used to “knock down” or inhibit a particular gene of interest by simply injecting, bathing, or feeding to the organism of interest the double-stranded RNA molecule. This technique selectively “knock downs” gene function without requiring transfection or recombinant techniques (Giet, 2001; Hammond, 2001; Stein P, et al., 2002; Svoboda P, et al., 2001; Svoboda P, et al., 2000).

Another type of RNAi is often referred to as small interfering RNA (siRNA), which may also be utilized to inhibit TEX14. A siRNA may comprises a double stranded structure or a single stranded structure, the sequence of which is “substantially identical” to at least a portion of the target gene (See WO 04/046320, which is incorporated herein by reference in its entirety). “Identity,” as known in the art, is the relationship between two or more polynucleotide (or polypeptide) sequences, as determined by comparing the sequences. In the art, identity also means the degree of sequence relatedness between polynucleotide sequences, as determined by the match of the order of nucleotides between such sequences. Identity can be readily calculated. See, for example: Computational Molecular Biology, Lesk, A. M., ed. Oxford University Press, New York, 1988; Biocomputing: Informatics and Genome Projects, Smith, D. W., ea., Academic Press, New York, 1993, and the methods disclosed in WO 99/32619, WO 01/68836, WO 00/44914, and WO 01/36646, specifically incorporated herein by reference. While a number of methods exist for measuring identity between two nucleotide sequences, the term is well known in the art. Methods for determining identity are typically designed to produce the greatest degree of matching of nucleotide sequence and are also typically embodied in computer programs. Such programs are readily available to those in the relevant art. For example, the GCG program package (Devereux et al.), BLASTP, BLASTN, and FASTA (Atschul et al.) and CLUSTAL (Higgins et al., 1992; Thompson, et al., 1994).

Thus, siRNA contains a nucleotide sequence that is essentially identical to at least a portion of the target gene, TEX14. More particularly, the siRNA molecule contains a nucleotide sequence that is essentially identical to at least a portion of SEQ ID NO:7, SEQ ID NO:8, SEQ ID NO:9, SEQ ID NO:10, SEQ ID NO:11, or SEQ ID NO:12. Preferably, the siRNA contains a nucleotide sequence that is completely identical to at least a portion of the target gene. Of course, when comparing an RNA sequence to a DNA sequence, an “identical” RNA sequence will contain ribonucleotides where the DNA sequence contains deoxyribonucleotides, and further that the RNA sequence will typically contain a uracil at positions where the DNA sequence contains thymidine.

One of skill in the art will appreciate that two polynucleotides of different lengths may be compared over the entire length of the longer fragment. Alternatively, small regions may be compared. Normally sequences of the same length are compared for a final estimation of their utility in the practice of the present invention. It is preferred that there be 100% sequence identity between the dsRNA for use as siRNA and at least 15 contiguous nucleotides of the target gene (e.g., TEX14), although a dsRNA having 70%, 75%, 80%, 85%, 90%, or 95% or greater may also be used in the present invention. A siRNA that is essentially identical to a least a portion of the target gene may also be a dsRNA wherein one of the two complementary strands (or, in the case of a self-complementary RNA, one of the two self-complementary portions) is either identical to the sequence of that portion or the target gene or contains one or more insertions, deletions or single point mutations relative to the nucleotide sequence of that portion of the target gene. siRNA technology thus has the property of being able to tolerate sequence variations that might be expected to result from genetic mutation, strain polymorphism, or evolutionary divergence.

There are several methods for preparing siRNA, such as chemical synthesis, in vitro transcription, siRNA expression vectors, and PCR expression cassettes. Irrespective of which method one uses, the first step in designing an siRNA molecule is to choose the siRNA target site, which can be any site in the target gene. In certain embodiments, one of skill in the art may manually select the target selecting region of the gene, which may be an ORF (open reading frame) as the target selecting region and may preferably be 50-100 nucleotides downstream of the “ATG” start codon. However, there are several readily available programs available to assist with the design of siRNA molecules, for example siRNA Target Designer by Promega, siRNA Target Finder by GenScript Corp., siRNA Retriever Program by Imgenex Corp., EMBOSS siRNA algorithm, siRNA program by Qiagen, Ambion siRNA predictor, Ambion siRNA predictor, Whitehead siRNA prediction, and Sfold. Thus, it is envisioned that any of the above programs may be utilized to produce siRNA molecules that can be used in the present invention.

3. Ribozymes

Ribozymes are RNA-protein complexes that cleave nucleic acids in a site-specific fashion. Ribozymes have specific catalytic domains that possess endonuclease activity (Kim and Cech, 1987; Forster and Symons, 1987). For example, a large number, of ribozymes accelerate phosphoester transfer reactions with a high degree of specificity, often cleaving only one of several phosphoesters in an oligonucleotide substrate (Cech et al., 1981; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence specific cleavage/ligation reactions involving nucleic acids (Joyce, 1989; Cech et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression is particularly suited to therapeutic applications (Scanlon et al., 1991; Sarver et al., 1990; Sioud et al., 1992). Most of this work involved the modification of a target mRNA, based on a specific mutant codon that is cleaved by a specific ribozyme. In light of the information included herein and the knowledge of one of ordinary skill in the art, the preparation and use of additional ribozymes that are specifically targeted to a given gene will now be straightforward.

Other suitable ribozymes include sequences from RNase P with RNA cleavage activity (Yuan et al., 1992; Yuan and Altman, 1994), hairpin ribozyme structures (Berzal-Herranz et al., 1992; Chowrira et al., 1993) and hepatitis □ virus based ribozymes (Perrotta and Been, 1992). The general design and optimization of ribozyme directed RNA cleavage activity has been discussed in detail (Haseloff and Gerlach, 1988; Symons, 1992; Chowrira, et al., 1994; and Thompson, et al., 1995).

The other variable on ribozyme design is the selection of a cleavage site on a given target RNA. Ribozymes are targeted to a given sequence by virtue of annealing to a site by complimentary base pair interactions. Two stretches of homology are required for this targeting. These stretches of homologous sequences flank the catalytic ribozyme structure defined above. Each stretch of homologous sequence can vary in length from 7 to 15 nucleotides. The only requirement for defining the homologous sequences is that, on the target RNA, they are separated by a specific sequence which is the cleavage site. For hammerhead ribozymes, the cleavage site is a dinucleotide sequence on the target RNA, uracil (U) followed by either an adenine, cytosine or uracil (A,C or U; Perriman, et al., 1992; Thompson, et al., 1995). The frequency of this dinucleotide occurring in any given RNA is statistically 3 out of 16.

Designing and testing ribozymes for efficient cleavage of a target RNA is a process well known to those skilled in the art. Examples of scientific methods for designing and testing ribozymes are described by Chowrira et al. (1994) and Lieber and Strauss (1995), each incorporated by reference. The identification of operative and preferred sequences for use in Chk2 targeted ribozymes is simply a matter of preparing and testing a given sequence, and is a routinely practiced screening method known to those of skill in the art.

D. Protein Variants

Amino acid sequence variants of TEX14 can be used as inhibitors of TEX14 activity. These variants can be substitutional, insertional and/or deletion variants, for example. These variants may be purified according to known methods, such as precipitation (e.g., ammonium sulfate), HPLC, ion exchange chromatography, affinity chromatography (including immunoaffinity chromatography) or various size separations (sedimentation, gel electrophoresis, gel filtration), for example.

Substitutional variants or replacement variants typically contain the exchange of at least one amino acid for another at one or more sites within the protein. Substitutions can be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

It is thus contemplated by the inventors that various changes may be made in the DNA sequences of genes without appreciable loss of their biological utility or activity, as discussed below. The activity being activation of AR transcription and/or cyclin D1 expression and/or c-Jun transcription, etc.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte and Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine −0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still obtains a biologically equivalent and immunologically equivalent protein. In such changes, the substitution of amino acids whose hydrophilicity values are within ±2 is preferred, those that are within +1 are particularly preferred, and those within ±0.5 are even more particularly preferred.

1. Fusion Proteins

A specialized kind of insertional variant is the fusion protein. This molecule generally has all or a substantial portion of the native molecule, linked at the N- or C-terminus, to all or a portion of a second polypeptide. For example, a fusion protein of the present invention can includes the addition of a protein transduction domains, for example, but not limited to Antennepedia transduction domain (ANTP), HSV1 (VP22) and HIV-1 (Tat). Fusion proteins containing protein transduction domains (PTDs) can traverse biological membranes efficiently, thus delivering the protein of interest into the cell.

Yet further, inclusion of a cleavage site at or near the fusion junction will facilitate removal of the extraneous polypeptide after purification. Other useful fusions include linking of functional domains, such as active sites from enzymes, glycosylation domains, other cellular targeting signals or transmembrane regions.

2. Domain Switching

An interesting series of variants can be created by substituting homologous regions of various proteins. This is known, in certain contexts, as “domain switching.”

Domain switching involves the generation of chimeric molecules using different but, in this case, related polypeptides. By comparing various TEX14 proteins, one can make predictions as to the functionally significant regions of these molecules. It is possible, then, to switch related domains of these molecules in an effort to determine the criticality of these regions to function of the protein. These molecules may have additional value in that these “chimeras” can be distinguished from natural molecules, while possibly providing the same function.

3. Synthetic Peptides

The present invention also describes smaller TEX14-related peptides for use in various embodiments of the present invention. Because of their relatively small size, the peptides of the invention can also be synthesized in solution or on a solid support in accordance with conventional techniques. Various automatic synthesizers are commercially available and can be used in accordance with known protocols. See, for example, Stewart and Young (1984); Tam et al. (1983); Merrifield (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

E. Antigen Compositions

The present invention also provides for the use of TEX14 proteins or polypeptides as antigens for the immunization of animals relating to the production of antibodies. It is envisioned that TEX14 proteins, polypeptides or portions thereof, will be coupled, bonded, bound, conjugated or chemically-linked to one or more agents via linkers, polylinkers or derivatized amino acids. This may be performed such that a bispecific or multivalent composition or vaccine is produced. It is further envisioned that the methods used in the preparation of these compositions will be familiar to those of skill in the art and should be suitable for administration to animals, i.e., pharmaceutically acceptable. Preferred agents are the carriers are keyhole limpet hemocyanin (KLH) or bovine serum albumin (BSA).

1. Antibody Production

In certain embodiments, the present invention provides antibodies that bind with high specificity to the TEX14 polypeptides provided herein. Thus, antibodies that bind to the polypeptide of SEQ.ID.NO:1, SEQ.ID.NO:2, SEQ.ID.NO:3, SEQ.ID.NO.4, SEQ.ID.NO.5 and/or SEQ.ID.NO.6 are provided. In addition to antibodies generated against the full length proteins, antibodies may also be generated in response to smaller constructs comprising epitopic core regions, including wild-type and mutant epitopes.

Monoclonal antibodies (MAbs) are recognized to have certain advantages, e.g., reproducibility and large-scale production, and their use is generally preferred. The invention thus provides monoclonal antibodies of the human, murine, monkey, rat, hamster, rabbit and even chicken origin. Due to the ease of preparation and ready availability of reagents, murine monoclonal antibodies will often be preferred.

However, humanized antibodies are also contemplated, as are chimeric antibodies from mouse, rat, or other species, bearing human constant and/or variable region domains, bispecific antibodies, recombinant and engineered antibodies and fragments thereof. Methods for the development of antibodies that are “custom-tailored” to the patient's dental disease are likewise known and such custom-tailored antibodies are also contemplated.

A polyclonal antibody is prepared by immunizing an animal with an immunogenic TEX14 composition in accordance with the present invention and collecting antisera from that immunized animal.

A wide range of animal species can be used for the production of antisera. Typically the animal used for production of antisera is a rabbit, a mouse, a rat, a hamster, a guinea pig or a goat. Because of the relatively large blood volume of rabbits, a rabbit is a preferred choice for production of polyclonal antibodies.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobenzoyl-N-hydroxysuccinimide ester, carbodiimide and bis-biazotized benzidine.

As is also well known in the art, the immunogenicity of a particular immunogen composition can be enhanced by the use of non-specific stimulators of the immune response, known as adjuvants. Suitable adjuvants include all acceptable immunostimulatory compounds, such as cytokines, toxins or synthetic compositions.

Adjuvants that may be used include IL-1, IL-2, IL-4, IL-7, IL-12, γ-interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion is also contemplated. MHC antigens may even be used. Exemplary, often preferred adjuvants include complete Freund's adjuvant (a non-specific stimulator of the immune response containing killed Mycobacterium tuberculosis), incomplete Freund's adjuvants and aluminum hydroxide adjuvant.

In addition to adjuvants, it may be desirable to coadminister biologic response modifiers (BRM), which have been shown to upregulate T cell immunity or downregulate suppressor cell activity. Such BRMs include, but are not limited to, Cimetidine (CIM; 1200 mg/d) (Smith/Kline, PA); low-dose Cyclophosphamide (CYP; 300 mg/m2) (Johnson/Mead, NJ), cytokines such as γ-interferon, IL-2, or IL-12 or genes encoding proteins involved in immune helper functions, such as B-7.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization.

A second, booster injection, may also be given. The process of boosting and titering is repeated until a suitable titer is achieved. When a desired level of immunogenicity is obtained, the immunized animal can be bled and the serum isolated and stored, and/or the animal can be used to generate MAbs.

For production of rabbit polyclonal antibodies, the animal can be bled through an ear vein or alternatively by cardiac puncture. The removed blood is allowed to coagulate and then centrifuged to separate serum components from whole cells and blood clots. The serum may be used as is for various applications or else the desired antibody fraction may be purified by well-known methods, such as affinity chromatography using another antibody, a peptide bound to a solid matrix, or by using, e.g., protein A or protein G chromatography.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified TEX14 protein, polypeptide, peptide or domain, be it a wild-type or mutant composition. The immunizing composition is administered in a manner effective to stimulate antibody producing cells.

The methods for generating monoclonal antibodies (MAbs) generally begin along the same lines as those for preparing polyclonal antibodies. Rodents such as mice and rats are preferred animals, however, the use of rabbit, sheep or frog cells is also possible. The use of rats may provide certain advantages (Goding, 1986, pp. 60-61), but mice are preferred, with the BALB/c mouse being most preferred as this is most routinely used and generally gives a higher percentage of stable fusions.

The animals are injected with antigen, generally as described above. The antigen may be coupled to carrier molecules such as keyhole limpet hemocyanin if necessary. The antigen would typically be mixed with adjuvant, such as Freund's complete or incomplete adjuvant. Booster injections with the same antigen would occur at approximately two-week intervals.

Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible.

Often, a panel of animals will have been immunized and the spleen of an animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, pp. 65-66, 1986; Campbell, 1984). For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions.

One preferred murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine-resistant mouse murine myeloma SP2/0 non-producer cell line.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods is also appropriate (Goding pp. 71-74, 1986). 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide MAbs. The cell lines may be exploited for MAb production in two basic ways. First, a sample of the hybridoma can be injected (often into the peritoneal cavity) into a histocompatible animal of the type that was used to provide the somatic and myeloma cells for the original fusion (e.g., a syngeneic mouse). Optionally, the animals are primed with a hydrocarbon, especially oils such as pristane (tetramethylpentadecane) prior to injection. The injected animal develops tumors secreting the specific monoclonal antibody produced by the fused cell hybrid. The body fluids of the animal, such as serum or ascites fluid, can then be tapped to provide MAbs in high concentration. Second, the individual cell lines could be cultured in vitro, where the MAbs are naturally secreted into the culture medium from which they can be readily obtained in high concentrations.

MAbs produced by either means may be further purified, if desired, using filtration, centrifugation and various chromatographic methods such as HPLC or affinity chromatography. Fragments of the monoclonal antibodies of the invention can be obtained from the monoclonal antibodies so produced by methods, which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It is also contemplated that a molecular cloning approach may be used to generate monoclonals. For this, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 10⁴ times as many antibodies can be produced and screened in a single round, and that new specificities are generated by H and L chain combination which further increases the chance of finding appropriate antibodies.

Alternatively, monoclonal antibody fragments encompassed by the present invention can be synthesized using an automated peptide synthesizer, or by expression of full-length gene or of gene fragments in E. coli.

2. Antibody Conjugates

The present invention further provides antibodies against TEX14, generally of the monoclonal type, that are linked to one or more other agents to form an antibody conjugate. Any antibody of sufficient selectivity, specificity and affinity may be employed as the basis for an antibody conjugate. Such properties may be evaluated using conventional immunological screening methodology known to those of skill in the art.

Certain examples of antibody conjugates are those conjugates in which the antibody is linked to a detectable label. “Detectable labels” are compounds or elements that can be detected due to their specific functional properties, or chemical characteristics, the use of which allows the antibody to which they are attached to be detected, and further quantified if desired. Another such example is the formation of a conjugate comprising an antibody linked to a cytotoxic or anti-cellular agent, as may be termed “immunotoxins” (described in U.S. Pat. Nos. 5,686,072, 5,578,706, 4,792,447, 5,045,451, 4,664,911 and 5,767,072, each incorporated herein by reference).

Antibody conjugates are thus preferred for use as diagnostic agents. Antibody diagnostics generally fall within two classes, those for use in in vitro diagnostics, such as in a variety of immunoassays, and those for use in vivo diagnostic protocols, generally known as “antibody-directed imaging.” Again, antibody-directed imaging is less preferred for use with this invention.

Many appropriate imaging agents are known in the art, as are methods for their attachment to antibodies (see, e.g., U.S. Pat. Nos. 5,021,236 and 4,472,509, both incorporated herein by reference). Certain attachment methods involve the use of a metal chelate complex employing, for example, an organic chelating agent such a DTPA attached to the antibody (U.S. Pat. No. 4,472,509). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention ²¹¹astatine, ¹⁴carbon, ⁵¹chromium, ³⁶chlorine, ⁵⁷cobalt, ⁵⁸cobalt, ⁶⁷copper, ¹⁵²Eu, ⁶⁷gallium, ³hydrogen, ¹²³iodine, ¹²⁵iodine, ¹³¹iodine, ¹¹¹indium, ⁵⁹iron, ³²phosphorus, ¹⁸⁶rhenium, ¹⁸⁸rhenium, ⁷⁵selenium, ³⁵sulphur, and ⁹⁹mtechnicium. 125I is often being preferred for use in certain embodiments, and ⁹⁹mtechniciumand ¹¹¹indium are also often preferred due to their low energy and suitability for long range detection.

Radioactively labeled monoclonal antibodies of the present invention may be produced according to well-known methods in the art. For instance, monoclonal antibodies can be iodinated by contact with sodium or potassium iodide and a chemical oxidizing agent such as sodium hypochlorite, or an enzymatic oxidizing agent, such as lactoperoxidase. Monoclonal antibodies according to the invention may be labeled with ⁹⁹mtechnetium by ligand exchange process, for example, by reducing pertechnate with stannous solution, chelating the reduced technetium onto a Sephadex column and applying the antibody to this column or by direct labeling techniques, e.g., by incubating pertechnate, a reducing agent such as SNC12, a buffer solution such as sodium-potassium phthalate solution, and the antibody. Intermediary functional groups which are often used to bind radioisotopes which exist as metallic ions to antibody are diethylenetriaminepentaacetic acid (DTPA) and ethylene diaminetetracetic acid (EDTA). Also contemplated for use are fluorescent labels, including rhodamine, fluorescein isothiocyanate and renographin.

The much preferred antibody conjugates of the present invention are those intended primarily for use in vitro, where the antibody is linked to a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. Preferred secondary binding ligands are biotin and avidin or streptavidin compounds. The use of such labels is well known to those of skill in the art in light and is described, for example, in U.S. Pat. Nos. 3,817,837; 3,850,752; 3,939,350; 3,996,345; 4,277,437; 4,275,149 and 4,366,241; each incorporated herein by reference.

### III. SCREENING FOR INHIBITORS

The present invention comprises methods for identifying inhibitors that directly or indirectly affect the activity and/or expression of TEX14 or that deleteriously affect the intercellular bridges that connect germ cells. These assays may comprise random screening of large libraries of candidate substances; alternatively, the assays may be used to focus on particular classes of compounds selected with an eye towards structural attributes that are believed to make them more likely to modulate the function or activity or expression of TEX14, for example. Yet further, the present invention encompasses inhibitors identified in U.S. Application No. 2002/0081592, which is incorporated herein by reference in its entirety.

By function, it is meant that one may assay for mRNA expression, protein expression, protein activity, binding activity, or ability to associate and/or dissociate from other members of the complex and otherwise determine functions contingent on the TEX14 proteins or nucleic acid molecules.

Without being bound to the description of inhibitors, the present invention also encompasses methods for identifying stimulators that affect the activity and/or expression of TEX14. These assays may comprise random screening of large libraries of candidate substances; alternatively, the assays may be used to focus on particular classes of compounds selected with an eye towards structural attributes that are believed to make them more likely to modulate the function or activity or expression of TEX14. Yet further, the present invention encompasses stimulators identified in U.S. Application No. 2002/0081592, which is incorporated herein by reference in its entirety. Stimulators can be used to enhance fertility. One of skill in the art is aware that the below procedures can be altered to identify compounds that result in the opposite effect of an inhibitor, such as increasing the activity and/or expression of TEX14.

A. Inhibitors

The present invention further comprises methods for identifying, making, generating, providing, manufacturing or obtaining inhibitors of TEX14 activity or expression. TEX14 nucleic acid or polypeptide may be used as a target in identifying compounds that inhibit, decrease or down-regulate its expression or activity in germ cells, such as spermatozoa, spermatids, spermatocytes and spermatogonia. In other embodiments, compounds screened for would decrease intercellular bridge development and/or increase apoptosis in germ cells and/or disruption of DNA synthesis. These assays may comprise random screening of large libraries of candidate substances; alternatively, the assays may be used to focus on particular classes of compounds selected with an eye towards structural attributes that are believed to make them more likely to inhibit the function of TEX14 molecules. By function, it is meant that one may assay for inhibition of activity of TEX14 in germ cells, maintenance/function of the intercellular bridge, or inhibition of expression of TEX14. Such assays may include for example luciferase reportor system in which luciferase activity is measured or in vitro assays that measure protease activity.

To identify, make, generate, provide, manufacture or obtain a TEX14 inhibitor, one generally will determine the activity of the TEX14 molecule in the presence, absence, or both of the candidate substance, wherein an inhibitor is defined as any substance that down-regulates, reduces, inhibits or decreases TEX14 expression or activity. For example, a method may generally comprise:

(a) providing a candidate substance suspected of decreasing TEX14 expression or activity; or maintenance or function of the intercellular bridge

(b) assessing the ability of the candidate substance to decrease TEX14 expression or activity; or maintenance or function of the intercellular bridge

(c) selecting a TEX14 inhibitor; and

(d) manufacturing the inhibitor.

In further embodiments, a TEX14 polypeptide or nucleic acid may be provided in a cell or a cell free system and the TEX14 polypeptide or nucleic acid may be contacted with the candidate substance. Next, an inhibitor is selected by assessing the effect of the candidate substance on TEX14 activity or TEX14 expression. Upon identification of the inhibitor, the method may further provide manufacturing of the inhibitor.

Still further, the screening system can be used to screen for compounds that inhibit intercellular bridge formation, function and/or maintenance. This screening system can comprise: providing a candidate substance suspected of inhibiting intercellular bridge formation, function and/or maintenance; selecting the compound by assessing the ability of the candidate substance to inhibit intercellular bridge formation, function and/or maintenance; and making the selected compound.

In the process of screening for compounds that inhibit bridge formation, function and/or maintenance, a method of enriching intercellular bridges may be employed. Methods of enriching intercellular bridges may comprise steps of isolating or collecting a mitochondrial fraction from a tissue and/or other biological sample (e.g., testis), incubating the mitochondrial fraction with a detergent to disrupt the mitochondrial membranes and any other organelle membranes and collecting the intercellular bridge fraction.

As used herein, the term “candidate substance” refers to any molecule that may potentially inhibit TEX14 activity, expression or function. Candidate compounds may include fragments or parts of naturally-occurring compounds or may be found as active combinations of known compounds which are otherwise inactive. The candidate substance can be a nucleic acid (e.g., antisense molecule, siRNA molecule), a polypeptide (e.g., antibodies), a small molecule, etc. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds.

One basic approach to search for a candidate substance is screening of compound libraries. One may simply acquire, from various commercial sources, small molecule libraries that are believed to meet the basic criteria for useful drugs in an effort to “brute force” the identification of useful compounds. Screening of such libraries, including combinatorially generated libraries, is a rapid and efficient way to screen a large number of related (and unrelated) compounds for activity. Combinatorial approaches also lend themselves to rapid evolution of potential drugs by the creation of second, third and fourth generation compounds modeled of active, but otherwise undesirable compounds. It will be understood that an undesirable compound includes compounds that are typically toxic, but have been modified to reduce the toxicity or compounds that typically have little effect with minimal toxicity and are used in combination with another compound to produce the desired effect.

In specific embodiments, a small molecule library that is created by chemical genetics may be screened to identify a candidate substance that may be a modulator of the present invention (Clemons et al., 2001; Blackwell et al., 2001). Chemical genetics is the technology that uses small molecules to modulate the functions of proteins rapidly and conditionally. The basic approach requires identification of compounds that regulate pathways and bind to proteins with high specificity. Small molecules are prepared using diversity-oriented synthesis, and the split-pool strategy to allow spatial segregation on individual polymer beads. Each bead contains compounds to generate a stock solution that can be used for many biological assays.

The most useful pharmacological compounds may be compounds that are structurally related to compounds that interact naturally with compounds that modulate TEX14 transcription or activity. Creating and examining the action of such molecules is known as “rational drug design,” and include making predictions relating to the structure of target molecules. Thus, it is understood that the candidate substance identified by the present invention may be a small molecule activator or any other compound (e.g., polypeptide or polynucleotide) that may be designed through rational drug design starting from known inhibitors of TEX14.

The goal of rational drug design is to produce structural analogs of biologically active target compounds. By creating such analogs, it is possible to fashion drugs that are more active or stable than the natural molecules, that have different susceptibility to alteration or that may affect the function of various other molecules. In one approach, one would generate a three-dimensional structure for a molecule similar to TEX14, and then design a molecule for its ability to interact with an TEX14-related molecule. This could be accomplished by X-ray crystallography, computer modeling or by a combination of both approaches. The same approach may be applied to identifying interacting molecules of TEX14.

It also is possible to use antibodies to ascertain the structure of a target compound or activator. In principle, this approach yields a pharmacore/lead compound upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

It will, of course, be understood that all the screening methods of the present invention are useful in themselves notwithstanding the fact that effective candidates may not be found. The invention provides methods for screening for such candidates, not solely methods of finding them.

B. In vitro Assays

In one embodiment, the invention is to be applied for the screening of compounds that bind to the TEX14 polypeptide or fragment thereof. The polypeptide or fragment may be either free in solution, fixed to a support, expressed in or on the surface of a cell. Either the polypeptide or the compound may be labeled, thereby permitting determining of binding.

In another embodiment, the assay may measure the inhibition of binding of TEX14 to a natural or artificial substrate or binding partner. Competitive binding assays can be performed in which one of the agents (TEX14, binding partner or compound) is labeled. Usually, the polypeptide will be the labeled species. One may measure the amount of free label versus bound label to determine binding or inhibition of binding.

Another technique for high throughput screening of compounds is described in WO 84/03564. Large numbers of small peptide test compounds are synthesized on a solid substrate, such as plastic pins or some other surface. The peptide test compounds are reacted with TEX14 and washed. Bound polypeptide is detected by various methods.

Purified TEX14 can be coated directly onto plates for use in the aforementioned drug screening techniques. However, non-neutralizing antibodies to the polypeptide can be used to immobilize the polypeptide to a solid phase. Also, fusion proteins containing a reactive region (preferably a terminal region) may be used to link the TEX14 active region to a solid phase.

C. In cyto Assays

Various cell lines containing wild-type or natural or engineered mutations in TEX14 gene can be used to study various functional attributes of TEX14 and how a candidate compound affects these attributes. Methods for engineering mutations are described elsewhere in this document, as are naturally-occurring mutations in TEX14 that lead to, contribute to and/or otherwise cause infertility. In such assays, the compound would be formulated appropriately, given its biochemical nature, and contacted with a target cell. Depending on the assay, culture may be required. The cell may then be examined by virtue of a number of different physiologic assays. Alternatively, molecular analysis may be performed in which the function of TEX14, or related pathways, may be explored.

In a specific embodiment, yeast two-hybrid analysis is performed by standard means in the art with the polypeptides of the present invention, i.e., TEX14. Two hybrid screen is used to elucidate or characterize the function of a protein by identifying other proteins with which it interacts. The protein of unknown function, herein referred to as the “bait” is produced as a chimeric protein additionally containing the DNA binding domain of GAL4. Plasmids containing nucleotide sequences which express this chimeric protein are transformed into yeast cells, which also contain a representative plasmid from a library containing the GAL4 activation domain fused to different nucleotide sequences encoding different potential target proteins. If the bait protein physically interacts with a target protein, the GAL4 activation domain and GAL4 DNA binding domain are tethered and are thereby able to act conjunctively to promote transcription of a reporter gene. If no interaction occurs between the bait protein and the potential target protein in a particular cell, the GAL4 components remain separate and unable to promote reporter gene transcription on their own. One skilled in the art is aware that different reporter genes can be utilized, including β-galactosidase, HIS3, ADE2, or URA3. Furthermore, multiple reporter sequences, each under the control of a different inducible promoter, can be utilized within the same cell to indicate interaction of the GAL4 components (and thus a specific bait and target protein). A skilled artisan is aware that use of multiple reporter sequences decreases the chances of obtaining false positive candidates. Also, alternative DNA-binding domain/activation domain components may be used, such as LexA. One skilled in the art is aware that any activation domain may be paired with any DNA binding domain so long as they are able to generate transactivation of a reporter gene. Furthermore, a skilled artisan is aware that either of the two components may be of prokaryotic origin, as long as the other component is present and they jointly allow transactivation of the reporter gene, as with the LexA system.

Two hybrid experimental reagents and design are well known to those skilled in the art (see The Yeast Two-Hybrid System by P. L. Bartel and S. Fields (eds.) (Oxford University Press, 1997), including the most updated improvements of the system (Fashena et al., 2000). A skilled artisan is aware of commercially available vectors, such as the Matchmaker™ Systems from Clontech (Palo Alto, Calif.) or the HybriZAP® 2.1 Two Hybrid System (Stratagene; La Jolla, Calif.), or vectors available through the research community (Yang et al., 1995; James et al., 1996). In alternative embodiments, organisms other than yeast are used for two hybrid analysis, such as mammals (Mammalian Two Hybrid Assay Kit from Stratagene (La Jolla, Calif.)) or E. coli (Hu et al., 2000).

In an alternative embodiment, a two hybrid system is utilized wherein protein-protein interactions are detected in a cytoplasmic-based assay. In this embodiment, proteins are expressed in the cytoplasm, which allows posttranslational modifications to occur and permits transcriptional activators and inhibitors to be used as bait in the screen. An example of such a system is the CytoTrap® Two-Hybrid System from Stratagene (La Jolla, Calif.), in which a target protein becomes anchored to a cell membrane of a yeast which contains a temperature sensitive mutation in the cdc25 gene, the yeast homologue for hSos (a guanyl nucleotide exchange factor). Upon binding of a bait protein to the target, hSos is localized to the membrane, which allows activation of RAS by promoting GDP/GTP exchange. RAS then activates a signaling cascade which allows growth at 37° C. of a mutant yeast cdc25H. Vectors (such as pMyr and pSos) and other experimental details are available for this system to a skilled artisan through Stratagene (La Jolla, Calif.). (See also, for example, U.S. Pat. No. 5,776,689, herein incorporated by reference).

Thus, in accordance with an embodiment of the present invention, there is a method of screening for a peptide which interacts with TEX14 comprising introducing into a cell a first nucleic acid comprising a DNA segment encoding a test peptide, wherein the test peptide is fused to a DNA binding domain, and a second nucleic acid comprising a DNA segment encoding at least part of TEX14, respectively, wherein at least part of TEX14 respectively, is fused to a DNA activation domain. Subsequently, there is an assay for interaction between the test peptide and the TEX14 polypeptide or fragment thereof by assaying for interaction between the DNA binding domain and the DNA activation domain. For example, the assay for interaction between the DNA binding and activation domains may be activation of expression of β-galactosidase.

An alternative method is screening of lambda gt11, lambda LZAP (Stratagene) or equivalent cDNA expression libraries with recombinant TEX14. Recombinant TEX14 or fragments thereof are fused to small peptide tags such as FLAG, HSV or GST. The peptide tags can possess convenient phosphorylation sites for a kinase such as heart muscle creatine kinase or they can be biotinylated. Recombinant TEX14 can be phosphorylated with ³²[P] or used unlabeled and detected with streptavidin or antibodies against the tags. Lambda gt11cDNA expression libraries are made from cells of interest and are incubated with the recombinant TEX14, washed and cDNA clones which interact with TEX14 isolated. Such methods are routinely used by skilled artisans. See, e.g., Sambrook (supra).

Another method is the screening of a mammalian expression library in which the cDNAs are cloned into a vector between a mammalian promoter and polyadenylation site and transiently transfected in cells. Forty-eight hours later the binding protein is detected by incubation of fixed and washed cells with a labeled TEX14. In this manner, pools of cDNAs containing the cDNA encoding the binding protein of interest can be selected and the cDNA of interest can be isolated by further subdivision of each pool followed by cycles of transient transfection, binding and autoradiography. Alternatively, the cDNA of interest can be isolated by transfecting the entire cDNA library into mammalian cells and panning the cells on a dish containing the TEX14 bound to the plate. Cells which attach after washing are lysed and the plasmid DNA isolated, amplified in bacteria, and the cycle of transfection and panning repeated until a single cDNA clone is obtained. See Seed et al., 1987 and Aruffo et al., 1987 which are herein incorporated by reference. If the binding protein is secreted, its cDNA can be obtained by a similar pooling strategy once a binding or neutralizing assay has been established for assaying supernatants from transiently transfected cells. General methods for screening supernatants are disclosed in Wong et al., (1985).

Another alternative method is isolation of proteins interacting with the TEX14 directly from cells. Fusion proteins of TEX14 with GST or small peptide tags are made and immobilized on beads. Biosynthetically labeled or unlabeled protein extracts from the cells of interest are prepared, incubated with the beads and washed with buffer. Proteins interacting with the TEX14 are eluted specifically from the beads and analyzed by SDS-PAGE. Binding partner primary amino acid sequence data are obtained by microsequencing. Optionally, the cells can be treated with agents that induce a functional response such as tyrosine phosphorylation of cellular proteins. An example of such an agent would be a growth factor or cytokine such as interleukin-2.

Another alternative method is immunoaffinity purification. Recombinant TEX14 is incubated with labeled or unlabeled cell extracts and immunoprecipitated with anti-TEX14 antibodies. The immunoprecipitate is recovered with protein A-Sepharose and analyzed by SDS-PAGE. Unlabelled proteins are labeled by biotinylation and detected on SDS gels with streptavidin. Binding partner proteins are analyzed by microsequencing. Further, standard biochemical purification steps known to those skilled in the art may be used prior to microsequencing.

Yet another alternative method is screening of peptide libraries for binding partners. Recombinant tagged or labeled TEX14 is used to select peptides from a peptide or phosphopeptide library which interact with the TEX14. Sequencing of the peptides leads to identification of consensus peptide sequences which might be found in interacting proteins.

D. In vivo Assays

The present invention also encompasses the use of various animal models. Thus, any identity seen between human and other animal TEX14 provides an excellent opportunity to examine the function of TEX14 in a whole animal system where it is normally expressed. By developing or isolating mutant cells lines that fail to express normal TEX14, one can generate models in mice that enable one to study the mechanism of TEX14 and its role in intercellular bridge formation in spermatogenesis.

Treatment of animals with test compounds will involve the administration of the compound, in an appropriate form, to the animal. Administration will be by any route the could be utilized for clinical or non-clinical purposes, including but not limited to oral, nasal, buccal, rectal, vaginal or topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood or lymph supply and intratumoral injection.

Determining the effectiveness of a compound in vivo may involve a variety of different criteria. Such criteria include, but are not limited to, increased fertility, decreased fertility or contraception.

In one embodiment of the invention, transgenic animals are produced which contain a functional transgene encoding a functional TEX14 polypeptide or variants thereof. Transgenic animals expressing TEX14 transgenes, recombinant cell lines derived from such animals and transgenic embryos may be useful in methods for screening for and identifying agents that induce or repress function of TEX14. Transgenic animals of the present invention also can be used as models for studying disease states.

In one embodiment of the invention, an TEX14 transgene is introduced into a non-human host to produce a transgenic animal expressing a human or murine TEX14 gene. The transgenic animal is produced by the integration of the transgene into the genome in a manner that permits the expression of the transgene. Methods for producing transgenic animals are generally described by Wagner and Hoppe (U.S. Pat. No. 4,873,191; which is incorporated herein by reference), Brinster et al., 1985; which is incorporated herein by reference in its entirety) and in “Manipulating the Mouse Embryo; A Laboratory Manual” 2nd edition (eds., Hogan, Beddington, Costantimi and Long, Cold Spring Harbor Laboratory Press, 1994; which is incorporated herein by reference in its entirety).

It may be desirable to replace the endogenous TEX14 by homologous recombination between the transgene and the endogenous gene; or the endogenous gene may be eliminated by deletion as in the preparation of “knock-out” animals. Typically, a TEX14 gene exon flanked by genomic sequences is electroporated into embryonic stem cells (ES cells). The ES cells are injected into a blastocyst and implanted into a host femaleThe chimeric progeny are mated and their progeny are screened for the genomic alteration in TEX14. Transgenic animals may also be made by microinjecting oocytes with a desired transgene. These microinjected eggs are implanted into a host female and the progeny are screened for presence of the transgene. Transgenic animals may be produced from the fertilized eggs from a number of animals including, but not limited to reptiles, amphibians, birds, mammals, and fish. Within a particularly preferred embodiment, transgenic mice are generated which overexpress TEX14 or express a mutant form of the polypeptide. Alternatively, the absence of an TEX14 in “knock-out” mice permits the study of the effects that loss of TEX14 protein has on a cell in vivo.

As noted above, transgenic animals and cell lines derived from such animals may find use in certain testing experiments. In this regard, transgenic animals and cell lines capable of expressing wild-type or mutant TEX14 may be exposed to test substances. These test substances can be screened for the ability to enhance wild-type TEX14 expression and or function or impair the expression or function of mutant TEX14.

E. Production of Inhibitors

In an extension of any of the previously described screening assays, the present invention also provide for methods of producing inhibitors of TEX14. The methods comprising any of the preceding screening steps followed by an additional step of “producing or manufacturing the candidate substance identified as an inhibitor of” the screened activity.

### IV. DIAGNOSING INFERTILITY

As discussed above, the present inventors have determined that alterations in the TEX14 gene are associated with infertility. Therefore, TEX14 genes may be employed as a diagnostic or prognostic indicator of infertility in general. More specifically, point mutations, deletions, insertions or regulatory perturbations will be identified. The present invention contemplates further the diagnosis of infertility detecting changes in the levels of TEX14 expression.

A. Genetic Diagnosis

One embodiment of the instant invention comprises a method for detecting variation in the expression of TEX14. This may comprise determining the level of TEX14 expressed, or determining specific alterations in the expressed product.

The biological sample can be tissue or fluid. Various embodiments include cells from the testes. Other embodiments include fluid samples such as seminal fluid.

Nucleic acids used are isolated from cells contained in the biological sample, according to standard methodologies (Sambrook et al., 1989). The nucleic acid may be genomic DNA or fractionated or whole cell RNA. Where RNA is used, it may be desired to convert the RNA to a complementary DNA (cDNA). In one embodiment, the RNA is whole cell RNA; in another, it is poly-A RNA. Normally, the nucleic acid is amplified.

Depending on the format, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994).

Following detection, one may compare the results seen in a given patient with a statistically significant reference group of normal patients and patients that have been diagnosed with infertility.

It is contemplated that other mutations in the TEX14 gene may be identified in accordance with the present invention by detecting a nucleotide change in particular nucleic acids (U.S. Pat. No. 4,988,617, incorporated herein by reference). A variety of different assays are contemplated in this regard, including but not limited to, fluorescent in situ hybridization (FISH; U.S. Pat. No. 5,633,365 and U.S. Pat. No. 5,665,549, each incorporated herein by reference), direct DNA sequencing, PFGE analysis, Southern or Northern blotting, single-stranded conformation analysis (SSCA), RNAse protection assay, allele-specific oligonucleotide (ASO, e.g., U.S. Pat. No. 5,639,611), dot blot analysis, denaturing gradient gel electrophoresis (e.g., U.S. Pat. No. 5,190,856 incorporated herein by reference), RFLP (e.g., U.S. Pat. No. 5,324,631 incorporated herein by reference) and PCR™-SSCP. Methods for detecting and quantitating gene sequences, such as mutated genes and oncogenes, in for example biological fluids are described in U.S. Pat. No. 5,496,699, incorporated herein by reference.

Yet further, it is contemplated that chip-based DNA technologies such as those described by Hacia et al. (1996) and Shoemaker et al. (1996) can be used for diagnosis of infertility. Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization. See also Pease et al., (1994); Fodor et al., (1991).

B. Immunodiagnosis

Antibodies can be used in characterizing the TEX14 content through techniques such as ELISAs and Western blot analysis. This may provide a prenatal screen or in counseling for those individuals seeking to have children.

The steps of various other useful immunodetection methods have been described in the scientific literature, such as, e.g., Nakamura et al., (1987). Immunoassays, in their most simple and direct sense, are binding assays. Certain preferred immunoassays are the various types of radioimmunoassays (RIA) and immunobead capture assay. Immunohistochemical detection using tissue sections also is particularly useful. However, it will be readily appreciated that detection is not limited to such techniques, and Western blotting, dot blotting, FACS analyses, and the like also may be used in connection with the present invention.

The antibody compositions of the present invention will find great use in immunoblot or Western blot analysis. The antibodies may be used as high-affinity primary reagents for the identification of proteins immobilized onto a solid support matrix, such as nitrocellulose, nylon or combinations thereof. In conjunction with immunoprecipitation, followed by gel electrophoresis, these may be used as a single step reagent for use in detecting antigens against which secondary reagents used in the detection of the antigen cause an adverse background. Immunologically-based detection methods for use in conjunction with Western blotting include enzymatically-, radiolabel-, or fluorescently-tagged secondary antibodies against the toxin moiety are considered to be of particular use in this regard. U.S. Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody or a biotin/avidin ligand binding arrangement, as is known in the art.

### V. METHODS FOR TREATING

The present invention contemplates the use of a modulator of TEX14 to enhance contraception of an animal. Animals that are treated include, but are not limited to mammals or avian, for example, mice, rats, or monkeys are used as experimental animal models. In specific embodiments, the present invention is used to treat humans. It is also envisioned that companion animals can be treated for infertility or the prophylactic compositions can be used as a contraceptive. Companion animals include, but are not limited to dogs, cats, horses, or birds.

The present invention involves the method of administering a composition to animal in an amount to result in contraception. Thus, contraception involves the administration of a compound in an effective amount such that the amount decreases conception. In the present invention, any modulation or decrease in conception is considered contraception.

In certain embodiments of the present invention, an effective amount of a inhibitor of TEX14 is administered to an animal to enhance or increase contraception by inhibiting intercellular bridge formation, maintenence, or function, or by increasing apoptosis and/or by disrupting meiotic DNA synthesis. Thus, an inhibitor of TEX14 can play a role in degradation of a specific factor that allows progression thru meiosis, and therefore a decrease in TEX14 activity can result in infertility by disrupting meiosis, for example.

An effective amount of an TEX14 inhibitor that may be administered to a cell includes a dose of about 0.1 μM to about 100 μM. More specifically, doses of an TEX14 inhibitor to be administered are from about 0.1 μM to about 10 μM; about 1 μM to about 5 μM; about 5 μM to about 10 μM; about 10 μM to about 15 μM; about 15 μM to about 20 μM; about 20 μM to about 30 μM; about 30 μM to about 40 μM; about 40 μM to about 50 μM; about 50 μM to about 60 μM; about 60 μM to about 70 μM; about 70 μM to about 80 μM; about 80 μM to about 90 μM; and about 90 μM to about 100 μM. Of course, all of these amounts are exemplary, and any amount in-between these points is also expected to be of use in the invention.

The effective amount or “therapeutically effective amount” of the inhibitor of TEX14 or related compounds thereof to be used is that amount effective to produce beneficial results, particularly with respect to contraception, in the recipient animal or patient. Such an amount may be initially determined by reviewing the published literature, by conducting in vitro tests or by conducting metabolic studies in healthy experimental animals, for example. Before use in a clinical setting, it may be beneficial to conduct confirmatory studies in an animal model, preferably a widely accepted animal model of the particular disease to be treated. Preferred animal models for use in certain embodiments are rodent models, which are preferred because they are economical to use and, particularly, because the results gained are widely accepted as predictive of clinical value.

As is well known in the art, a specific dose level of active compounds such as a TEX14 inhibitor or related compounds thereof for any particular patient depends upon a variety of factors including the activity of the specific compound employed, the age, body weight, general health, sex, diet, time of administration, route of administration, rate of excretion, drug combination, and the presence of any disease undergoing therapy. The person responsible for administration will determine the appropriate dose for the individual subject. Moreover, for human administration, preparations should meet sterility, pyrogenicity, general safety and purity standards as required by FDA Office of Biologics standards.

A therapeutically effective amount of an TEX14 inhibitor or related compounds thereof as a treatment varies depending upon the host treated and the particular mode of administration. In one embodiment of the invention the dose range of the TEX14 inhibitor or related compounds thereof will be about 0.5 mg/kg body weight to about 500 mg/kg body weight. The term “body weight” is applicable when an animal is being treated. When isolated cells are being treated, “body weight” as used herein should read to mean “total cell body weight”. The term “total body weight” may be used to apply to both isolated cell and animal treatment. All concentrations and treatment levels are expressed as “body weight” or simply “kg” in this application are also considered to cover the analogous “total cell body weight” and “total body weight” concentrations. However, those of skill will recognize the utility of a variety of dosage range, for example, 1 mg/kg body weight to 450 mg/kg body weight, 2 mg/kg body weight to 400 mg/kg body weight, 3 mg/kg body weight to 350 mg/kg body weight, 4 mg/kg body weight to 300 mg/kg body weight, 5 mg/kg body weight to 250 mg/kg body weight, 6 mg/kg body weight to 200 mg/kg body weight, 7 mg/kg body weight to 150 mg/kg body weight, 8 mg/kg body weight to 100 mg/kg body weight, or 9 mg/kg body weight to 50 mg/kg body weight. Further, those of skill will recognize that a variety of different dosage levels will be of use, for example, 1 mg/kg, 2 mg/kg, 3 mg/kg, 4 mg/kg, 5 mg/kg, 7.5 mg/kg, 10 mg/kg, 12.5 mg/kg, 15 mg/kg, 17.5 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 120 mg/kg, 140 mg/kg, 150 mg/kg, 160 mg/kg, 180 mg/kg, 200 mg/kg, 225 mg/kg, 250mg/kg, 275mg/kg, 300 mg/kg, 325 mg/kg, 350 mg/kg, 375 mg/kg, 400 mg/kg, 450 mg/kg, 500 mg/kg, 550 mg/kg, 600 mg/kg, 700 mg/kg, 750 mg/kg, 800 mg/kg, 900 mg/kg, 1000 mg/kg, 1250 mg/kg, 1500 mg/kg, 1750 mg/kg, 2000 mg/kg, 2500 mg/kg, and/or 3000 mg/kg. Of course, all of these dosages are exemplary, and any dosage in-between these points is also expected to be of use in the invention. Any of the above dosage ranges or dosage levels may be employed for an TEX14 inhibitor or related compounds thereof.

Administration of the therapeutic TEX14 inhibitor composition of the present invention to a patient or subject will follow general protocols for the administration of contraceptives, taking into account the toxicity, if any, of the TEX14 inhibitor.

The treatments may include various “unit doses.” Unit dose is defined as containing a predetermined quantity of the therapeutic composition (an TEX14 inhibitor or its related compounds thereof) calculated to produce the desired responses in association with its administration, i.e., the appropriate route and treatment regimen. The quantity to be administered, and the particular route and formulation, are within the skill of those in the clinical arts. Also of import is the subject to be treated, in particular, the state of the subject and the protection desired.

### VI. FORMULATIONS AND ROUTES FOR ADMINISTRATION TO PATIENTS

Where clinical applications are contemplated, it will be necessary to prepare pharmaceutical compositions—expression vectors, virus stocks, proteins, antibodies and drugs, for example—in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral, nasal, buccal, rectal, vaginal, testicular, or topical. Alternatively, administration may be by orthotopic, intradermal, subcutaneous, intramuscular, intraperitoneal or intravenous injection. Such compositions would normally be administered as pharmaceutically acceptable compositions, described supra.

The active compounds also may be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the polypeptides of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient also may be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### VII. KITS OF THE INVENTION

Any of the compositions described herein may be comprised in a kit. In a non-limiting example, any composition of the invention may be comprised in a kit. The kits will thus comprise, in suitable container means, a composition that disrupts intercellular bridges that interconnect germ cells, a TEX14 binding partner, and/or a TEX14 inhibitor and, optionally a lipid, and/or an additional agent of the present invention.

The kits may comprise a suitably aliquoted composition of the present invention, whether labeled or unlabeled, as may be used to prepare a standard curve for a detection assay. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing a composition of the invention and any other reagent containers in close confinement for commercial sale. Such containers may include injection or blow molded plastic containers into which the desired vials are retained.

However, the components of the kit may be provided as dried powder(s). When reagents and/or components are provided as a dry powder, the powder can be reconstituted by the addition of a suitable solvent. It is envisioned that the solvent may also be provided in another container means.

The container means will generally include at least one vial, test tube, flask, bottle, syringe and/or other container means, into which the composition is placed, preferably, suitably allocated. The kits may also comprise a second container means for containing a sterile, pharmaceutically acceptable buffer and/or other diluent.

The kits of the present invention may also typically include a means for containing the vials in close confinement for commercial sale, such as, e.g., injection and/or blow-molded plastic containers into which the desired vials are retained.

Irrespective of the number and/or type of containers, the kits of the invention may also comprise, and/or be packaged with, an instrument for assisting with the injection/administration and/or placement of the ultimate composition within the body of an animal. Such an instrument may be a syringe, pipette, forceps, and/or any such medically approved delivery vehicle, for example.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### mRNA Expression Analysis

RNA isolation and Northern blot analysis were carried out as described previously (Dong et al. 1996). The cDNA probes used for analysis are as follows: cyclin A1 (261-751 bp of NM_{—}007628); Kit (1050-1512 bp of Y00864); Gapd (442-728 bp of NM_{—}001001303), and Tex14 (144-445 bp of BK000966).

### Example 2

### Generation of Tex14 Mutant Mice

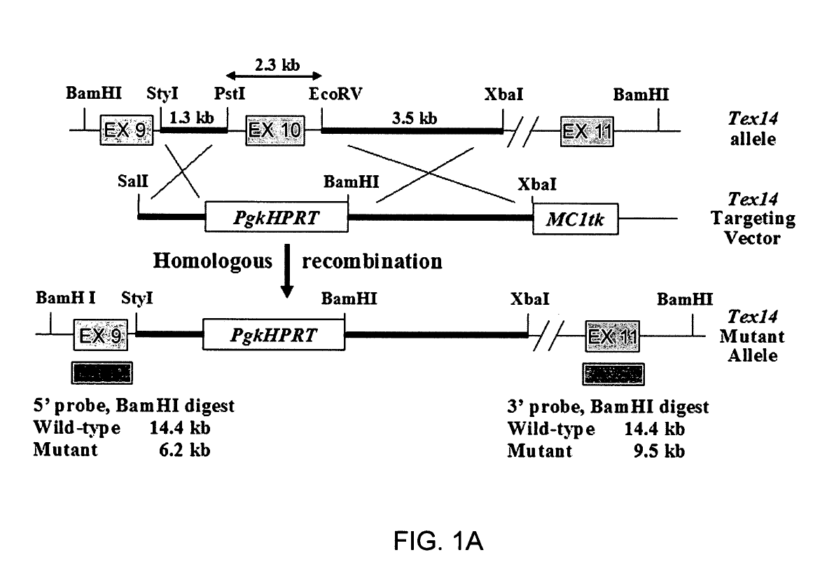

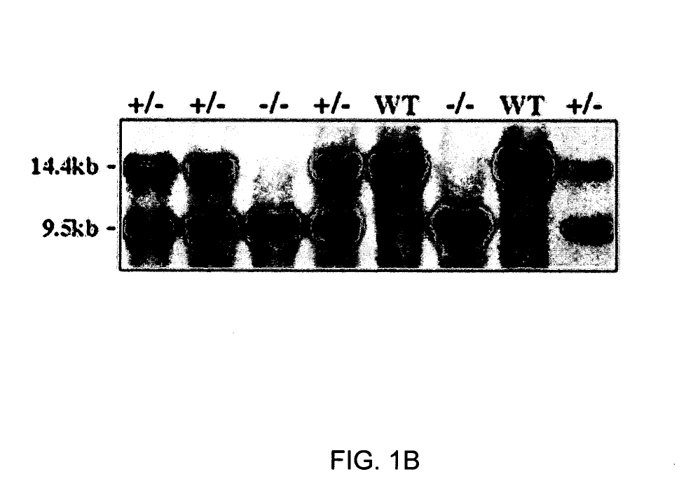

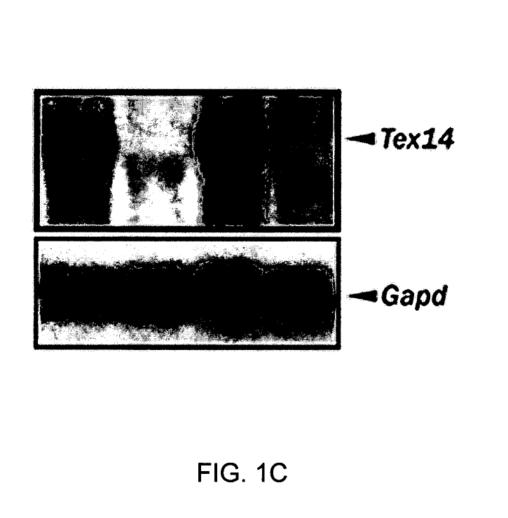

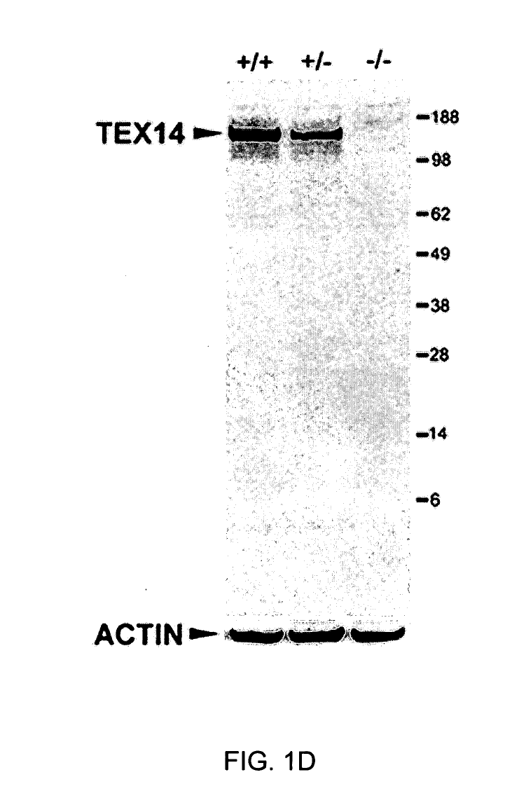

An in silico analysis was used to search for germ cell-specific genes. In this approach, several genes were discovered on mouse chromosome 11 that were preferentially expressed in the male testis including testis expressed gene 14 (Tex14) (Wu et al., 2003). To define the roles of TEX14 in mammalian development, a targeted deletion of Tex14 exon 11 (Tex14^{tm1Zuk}; herein called Tex14⁻) was produced in embryonic stem (ES) cells (FIG. 1A

More specifically, linearized Tex14 targeting vector (FIG. 1A) was electroporated into the HPRT-negative AB2.2 ES cell line, selected clones in hypoxanthine, aminopteridine, thymidine, and 1-(2′-deoxy-2′fluoro-β-D-arabinofuranosyl)-5-iodouracil, and screened the ES cell DNA by Southern blot as described by Matzuk et al. (1992). Correctly targeted clones were identified using 5′ and 3′ probes as shown (FIG. 1A). Mice were genotyped by Southern blot (Matzuk et al., 1992).

Heterozygous (Tex14^{±}) mice from 4 independent ES cell lines were viable and fertile over 6 months of breeding [7.14±0.56 pups/litter; 1.13±0.04 litters/month; n=20 mating pairs]. Chi-squared analysis of 349 F₂ offspring from these intercrosses (FIG. 1B and Table 1) demonstrated a statistically significant loss of homozygotes (Tex14^{-/-}) in the perinatal and postnatal periods [107 wild-type (30.66%); 184 heterozygotes (52.72%) and 58 homozygotes (16.62%); p<0.001]. Whereas Tex14^{-/-} females that survived to the adult stage were essentially normal and were fertile, grossly normal Tex14^{-/-} 129S6/SvEv inbred males (n=5) or C57B16/J/129S6SvEv hybrid males (n=12) from all 4 ES cell lines were infertile when bred to control females over a 6-12 month period. Absence of the Tex14 mRNA and protein in Tex14^{-/-} mice by Northern blot (FIG. 1C) and Western blot (FIG. 1D) analyses, respectively, confirmed that the Tex14 mutation was null. Thus, the generated Tex14 null mice showed that TEX14 is required for male fertility.

Table 1 shows that results of genotyping 169 female and 461 male progeny show homozygotes are lower than expected for female (p=0.004), male (p=0.006), and combined progeny (p=0.0006) by Chi-square analysis.

TABLE 1

| | Female Progeny | | | Male Progeny | | |
|---|---|---|---|---|---|---|
| ES cell line | +/+ | +/− | −/− | +/+ | +/− | −/− |
| 182-B11 | 19 | 28 | 8 | 41 | 85 | 23 |
| 182-C5 | 12 | 31 | 8 | 39 | 73 | 27 |
| 182-G5 | 9 | 15 | 4 | 29 | 37 | 21 |
| 182-C10 | 12 | 19 | 4 | 28 | 40 | 18 |
| Totals | 52 | 93 | 24 | 137 | 235 | 89 |

| | |
|---|---|
| 169 total females counted | 461 Total males counted |

| | Combined Progeny | | |
|---|---|---|---|
| ES cell line | +/+ | +/− | −/− |
| 182-B11 | 60 | 113 | 31 |
| 182-C5 | 51 | 104 | 35 |
| 182-G5 | 38 | 52 | 25 |
| 182-C10 | 40 | 59 | 22 |
| Totals | 189 | 328 | 113 |

630 total progeny counted

Progeny were born from Tex14^{-/-} intercrosses of lines derived four independent ES cell clones.

### Example 3

### Production of Anti-TEX14 Antibody

A cDNA fragment containing sequences encoding amino acids 885-1301 of the mouse TEX14 protein was subcloned into pET23b (Novagen), His-tagged TEX14 was produced in BL21 [DE3] pLysS cells, and polyclonal antibodies were raised in guinea pigs (Cocalico Biologicals, Reamstown, Pa.).

### Example 4

### Western Blot Analysis

Each lane was loaded with 50 ug of total testis lysate. The membrane was probed with the guinea pig anti-TEX14 polyclonal antibody at 1:2500. After developing, the membrane was stripped and reprobed with anti B3-actin clone AC-15 (Sigma, St. Louis, Mo.) at 1:3000. Secondary anti-guinea pig and anti-mouse horseradish peroxidase conjugated antibodies (Jackson Immuno Research Laboratories, West Grove, Pa.) were used at 1:10,000.

### Example 5

### Electron Microscopy

Testis from 11 day-old control and mutant mice were removed and small tears were made in the tunica albuginea. Testis were immediately submerged in 5 ml of 3% glutaraldehyde buffered to pH7.2 with 0.01M PIPES for 2 hours at room temperature. The tissue was post-fixed in PIPES buffered osmium tetroxide, pH 7.2, for one hour at room temperature, dehydrated and embedded. 60 nm sections were cut and mounted on copper grids (300 mesh). The grids were stained with uranyl acetate and lead citrate and examined on a Hitachi H7500, Gatan 2K×2K CCD. Bridges were counted for one hour from 10 sections from two Tex 14^{±} control testes and for six hours from 30 sections from three Tex14^{-/-} testes.

### Example 6

### Testosterone Measurements

Blood was drawn by cardiac puncture from six mutant and three control one year-old mice. Serum testosterone measurements were performed at the University of Virginia Center for Research in Reproduction Ligand Assay and Analysis Core.

### Example 7

### Histological Analyses

Testes were fixed in Bouin's or glutaraldehyde/paraformaldehyde, processed, and stained (Dong 1996; Kumar et al., 1997). Samples for immunohistochemistry were fixed in 4% paraformadehyde and embedded in paraffin. Five μm sections were boiled in 0.1M Na Citrate solution pH6 for antigen retrieval. Slides were incubated with guinea pig anti-TEX14 (1:2000), rabbit anti-SCP3 (1:1000), or rabbit anti-cyclin A1 (1:1000). Anti-guinea pig or anti-rabbit biotin conjugated secondary antibodies (Vector Laboratories, Burlingame, Calif.) were used at 1:200. Staining was visualized with DAB using Vectastain ABC according to the manufacturer (Vector Laboratories, Burlingame, Calif.). Samples for immunofluorescence were fixed in 4% paraformaldehyde and cryoprotected in 30% sucrose in phosphate buffered saline. Ten μm frozen sections were incubated with guinea pig anti-TEX14 (1:2000). Cy3 conjugated anti-guinea pig antibody (Jackson Immuno Research Laboratories, West Grove, Pa.) was used at 1:500. Actin was stained with 33 nM Alexa 488 phalloidin (Molecular Probes, Eugene, Oreg.). Sections were mounted with Vectashield mounting media with DAPI (Vector Laboratories, Burlingame, Calif.).

Whole mounts of seminiferous tubules were prepared as described (Rosen and Beddington, 1993; Ohmura et al., 2003) with minor modifications. Tubules for 25 day-old control and mutant testis were fixed in 4% PFA overnight at 4 degrees, washed twice with TBS, and serially dehydrated with 25%, 50%, 75%, and 100% TBST. Tubules were rehydrated and incubated in 5% goat serum in TBST for 1 hr at RT. The tubules were then incubated with a mouse anti-PLZF antibody (1:500, OP-128, Oncogene Research, Madison, Wis.) in TBS with 1% goat serum at 4 degrees overnight, washed 3 times in TBST for 5 minutes each, and incubated with goat anti-mouse Alexa 488 secondary antibody (1:1000) for 1 hour at RT. Tubules were washed as before and mounted in Vectashield with DAPI (Vector Laboratories). Images were taken using a Nikon Eclipse E600 microscope.

Terminal deoxynucleotidyltransferase-mediated dUTP-biotin nick end labeling (TUNEL) analysis of 4% paraformaldehyde-fixed testes was performed using the ApoTag Plus peroxidase kit according to the manufacturer (Intergen, Purchase, N.Y.). DNA laddering was performed (Yan et al., 2000). For 5-bromo-2-deoxyuridine (BrdU) analysis (Yan et al, 2000), 0.2 ml or 0.25 ml of 5 mg/ml BrdU was injected into 10 day and 14 day-old males, respectively. Mice were sacrificed at 1 hr post injection, and testes were fixed in 4% paraformaldehyde. Cells with labeled DNA were identified by immunohistochemisty using mouse clone Bu20a anti-BrdU antibody (DakoCytomation, Carpinteria, Calif.).

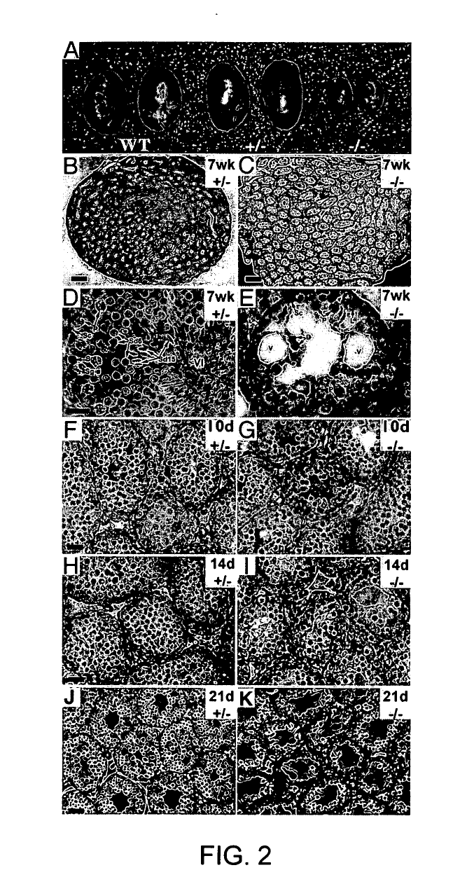

Testes from adult and juvenile Tex14^{-/-} males were significantly smaller (p<0.01) than Tex14^{±} or wild-type (WT) littermates (FIG. 2A). Six week-old Tex14^{-/-} testes (23.2±1.1 mg; n=14) were 26% the weight of wild-type (89.3±2.7 mg; n=11) and Tex14^{±} (89.9±2.7 mg; n=10) testes. Whereas wild-type and Tex14^{±} testes from adult males demonstrated robust spermatogenesis (FIGS. 2B, 2D), the seminiferous tubules of Tex14^{-/-} males lacked late meiotic (i.e., late pachytene and diplotene spermatocytes) and post-meiotic germ cells (i.e., spermatids and spermatozoa; FIGS. 2C, 2E) and showed a degenerative pattern similar to human “Sertoli cell only” syndrome. On, closer inspection, spermatogonia and early meiotic (pre-leptotene, leptotene, and zygotene) spermatocytes were evident, but pachytene spermatocytes were rarely observed in contrast to their abundance in all tubules of wild-type testes. There was no difference (P>0.05) between serum testosterone levels of Tex14^{-/-} (60.8±7.2) and Tex14^{±} (44.7±11.2) males.

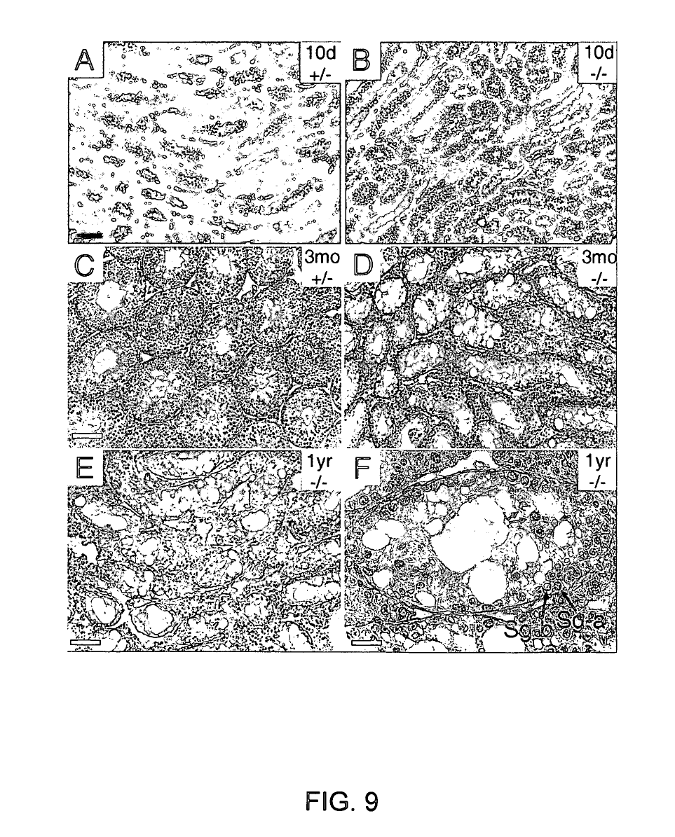

Developmentally, spermatogenesis can be divided into an initiation phase (before 6 weeks of age) and a maintenance phase (after 6 weeks of age). The defects in the absence of TEX14 could occur at both phases. To determine the onset of the disruption in the absence of TEX14, testes of younger mice at 10, 14, and 21 days of age were examined. Tex14^{±} and Tex14^{-/-} testes at 10 days were similar at the gross and histologic levels (FIGS. 2F, 2G, and FIG. 9). At 14 days, Tex14^{-/-} testes (FIG. 2I) contained fewer spermatocytes, as compared with wild-type testes (FIG. 2H), and early signs of Sertoli cell vacuolization (caused by severe germ cell depletion through endocytosis) can be observed. By 21 days of age, Tex14^{-/-} testes displayed severe degeneration and depletion of spermatocytes (FIG. 2K), and Sertoli cell vacuolization was more evident. Numerous round spermatids are present in the epithelium in Tex14^{±} testis (FIGS. 2D, 2J), whereas no spermatids and drastically reduced numbers of spermatocytes (mainly preleptotene, leptotene, and few zygotene spermatocytes) were visible in the Tex14^{-/-} testis. Thus, absence of TEX14 led to an early meiotic block during the first cycle of spermatogenesis in post-natal testes. Examination of older animals reveals an increasingly severe phenotype (FIG. 9). At one year, haploid cells remain absent, while spermatogonia and rare, dying spermaticoyes are still present in Tex14^{-/-} testes (FIGS. 9E, 9F) despite the overt decrease in germ cells.

### Example 8

### Protein Kinase Assays

FLAG-TEX14 form CMV 2A (Stratagene, La Jolla, Calif.) was expressed in HEK293 cells. Cells were lysed with 1% Triton X-100 in TBS and FLAG-TEX14 was immunoprecipitated with Anti-FLAG M2 beads (Sigma, St. Louis, Mo.) for 2 hours at 4 degrees. Beads were washed and FLAG-TEX14 was eluted into either TBS or the appropriate kinase buffer with 3×FLAG peptide (Sigma, St. Louis, Mo.). An equal volume of eluate from an IP of cells tranfected with empty CMV 2A vector was used as a negative control. Protein kinase activity was assayed with a Tyrosine Kinase Assay Kit (Upstate, Lake Placid, N.Y.) and Phospho-Serine/Throenine Assay Kit (Upstate, Lake Placid, N.Y.). Src and MAP Kinase 2 (Upstate, Lake Placid, N.Y.) were used as positive controls.

### Example 9

### mRNA Analysis

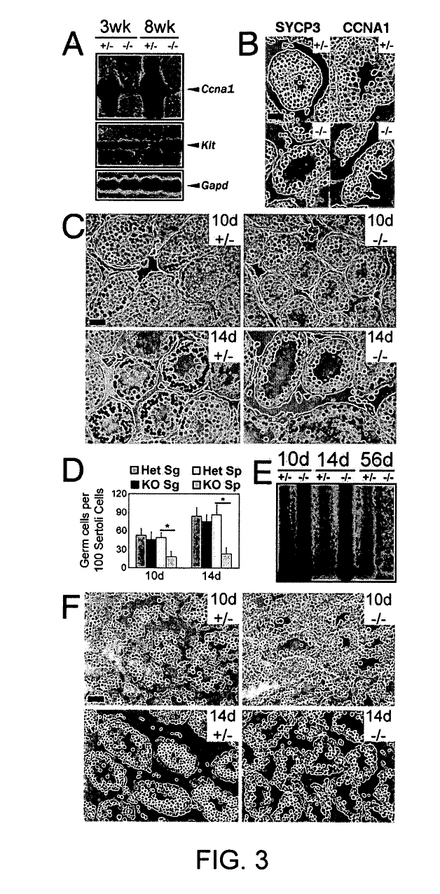

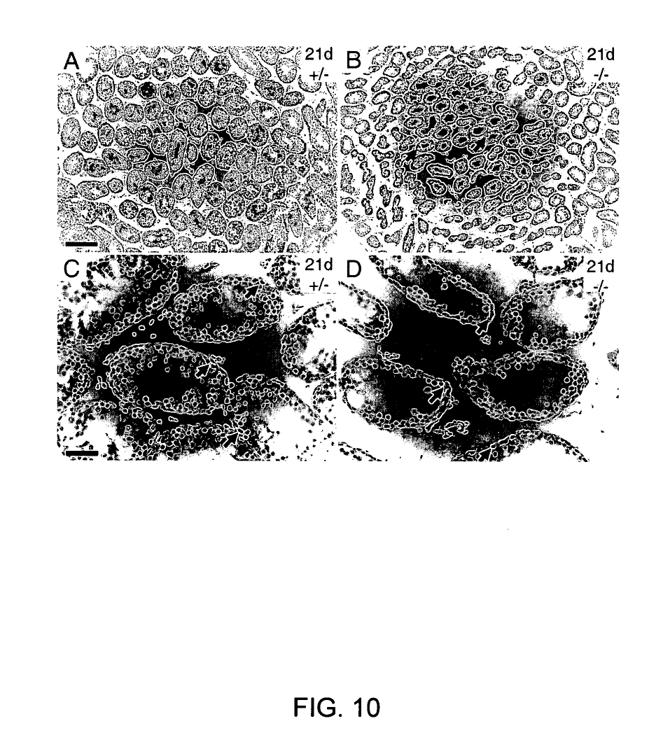

To further confirm the histological findings, several genes were analyzed that are expressed in spermatogonia or spermatocytes. Ccnal mRNA (encoding Cyclin A1, a marker for pachytene spermatocytes (Sweeney et al., 1996, Ravnik et al., 1999)) was abundant in Tex14^{±} testes at 3 weeks and 8 weeks of age but was barely detectable in Tex14^{-/-} testes of the same ages (FIG. 3A), showing that Tex14^{-/-} testes contained very few pachytene spermatocytes as early as 3 weeks. In contrast, mRNA levels of Kit (FIG. 2A), a marker for spermatogonia (Rossi et al., 2000), remained unchanged in 3 week-old Tex14^{-/-} testes and were higher in 8 week-old Tex14^{-/-} testes (caused by relative enrichment of spermatogonia due to severe depletion of spermatocytes and absence of haploid populations), supporting the observation that spermatogonia were maintained in Tex14^{-/-} testes. No decrease was seen in the number of PLZF-positive cells (FIGS. 10C, 10D), a marker of A single (Aₛ), A paired (Aₚᵣ), and A aligned (Aₐₗ) spermatagonia. Thus, at both the molecular and histological levels, at least some spermatogonial stem cells in Tex14^{-/-} males are able to undergo transit-amplification and enter meoisis I.

### Example 10

### Immunohistochemistry

Using immunohistochemistry, numerous Cyclin A1-positive (pachytene) spermatocytes were detected in Tex14^{±} testes (FIG. 3B). In contrast, rare Cyclin A1-positive cells were observed in Tex14^{-/-} testes (FIG. 3B and FIGS. 10A, 10B), which was consistent with the Northern blot and histologic analyses. An antibody to synaptonemal complex protein 3 (SCP3), a component of the synaptonemal complex and a marker for all primary spermatocytes (Moens et al., Dobson et al., 1994), labelled fewer SCP3-positive spermatocytes in the Tex14^{-/-} testes than in the Tex14^{±} testes (FIG. 3B), confirming that Tex14^{-/-} testes contained few pachytene spermatocytes and drastically reduced number of early spermatocytes including preleptotene, leptotene, and zygotene spermatocytes. Thus, at both the molecular and histological levels, TEX14 was required during the early meiotic phase of spermatogenesis.

### Example 11

### Reduced DNA Synthesis

To more precisely define the onset of the defects and potential causes of the disruption in the absence of TEX14, in vivo incorporation of BrdU (BrdU injection at 1 hour before sacrifice) was used in conjunction with immunohistochemical detection of BrdU-positive cells to quantify the number of S-phase spermatogonia and preleptotene spermatocytes (primary spermatocytes undergoing active meiotic DNA synthesis). By counting the number of BrdU-positive spermatogonia and spermatocytes in Tex14^{-/-} and Tex14^{±} testes at postnatal days 10 and 14, a significant reduction in the number of BrdU-positive spermatocytes was found (mostly pre-leptotene spermatocytes and possibly some very early leptotene spermatocytes) (FIGS. 3C and D). A slight, but not statistically significant, decrease in the number of spermatogonia was also observed in Tex14^{-/-} testes. This latter result was consistent with the Kit mRNA analysis, indicating spermatogonial populations were not severely affected in the absence of TEX14. The reduced number of DNA synthesizing preleptotene spermatocytes may reflect decreased proliferative activity or enhanced apoptosis.

### Example 12

### Apoptosis

To determine whether the reduced number of early spermatocytes was due to enhanced apoptosis, a TUNEL assay (FIG. 3F) was performed. In the Tex14^{-/-} testes at postnatal days 10 and 14, there was enhanced spermatocyte apoptosis since TUNEL-positive spermatocytes were present in almost every tubule in contrast to small clusters of TUNEL-positive spermatocytes that were occasionally seen in a few tubules in Tex14^{±} testes. To confirm the TUNEL assay results, DNA from the Tex14^{±} and Tex14^{-/-} testes was isolated at postnatal days 10, 14, and 56, and an apoptosis DNA ladder assay was performed (FIG. 3E). The internucleosomal cleavage of DNA was a hallmark of apoptosis, which can be demonstrated as a ladder of discrete 185-200 bp multimeric bands after agarose gel electrophoresis. The Tex14^{-/-} testes displayed quantitatively enhanced apoptotic DNA ladder patterns at both 10 and 14 days, as compared with Tex14^{±} littermates (FIG. 3E). At 56 days, the Tex14^{-/-} testes showed decreased levels of apoptotic DNA laddering, reflecting an absence of apoptotic germ cells due to a depletion of the degenerating germ cells. Thus, enhanced apoptosis of spermatocytes contributed to the decreased number of early spermatocytes in the Tex14^{-/-} testes. However, this can be the cause, or the consequences of disruption of meiotic DNA synthesis, or some other aspects of the cellular functions in the absence of TEX14.

### Example 13

### Subcellular Localization of TEX14

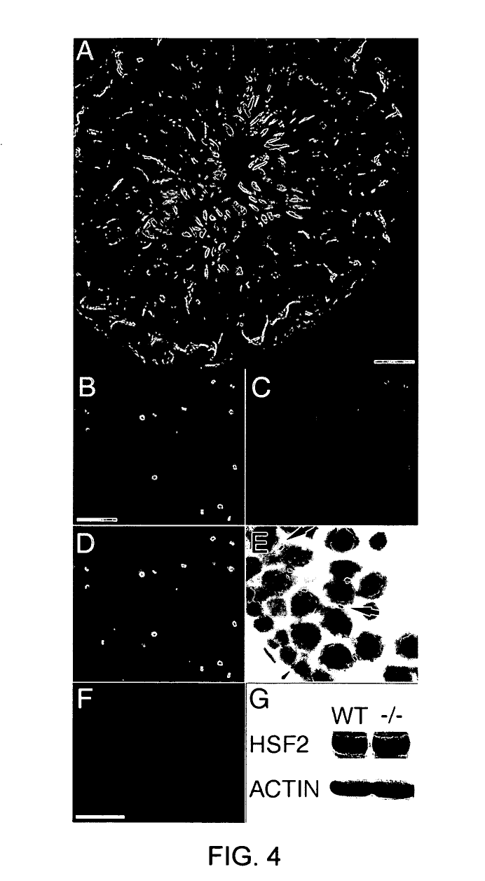

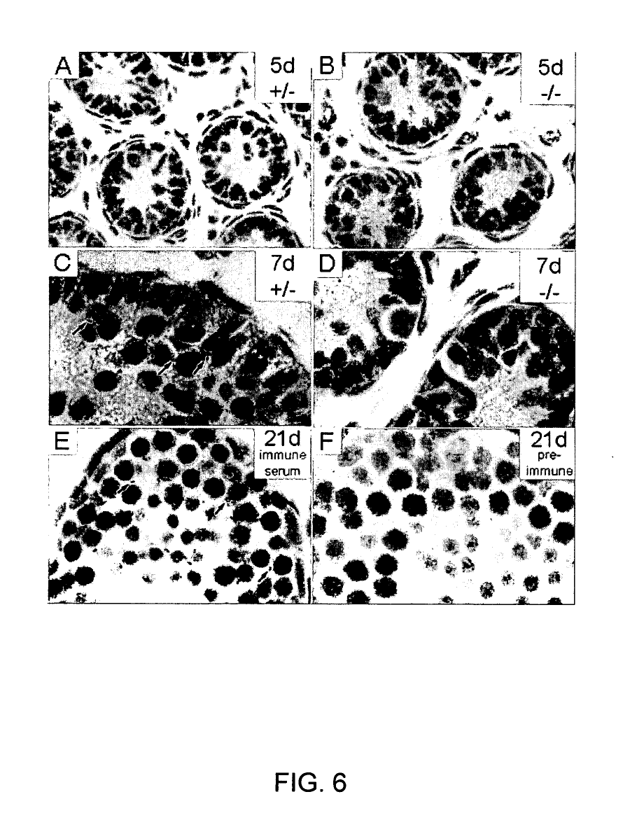

TEX14 is a large protein (1,450 aa; predicted molecular weight of 162.5 kD) with 3 N-terminal ankyrin repeats, a central kinase-like domain, and a C-terminal domain with limited homology to known proteins (Wu et al., 2003). To determine the subcellular localization of TEX14, polyclonal antibodies to TEX14 were generated. Western blot analysis demonstrated that the antibody was specific, detecting a ˜160 kDa protein in wild-type testes but no protein in the testes of Tex14^{-/-} mice (FIG. 1D and FIG. 6). By immunohistochemistry, TEX14 was detected as a “ring” of protein that bridged spermatids, spermatocytes, and some spermatogonia (FIGS. 4A, 4E). TEX14 could be seen to localize to intercellular bridges (ring canals) between spermatogonia as early as post-natal day 7 (FIGS. 6A, 6C).

### Example 14

### Localization of TEX14 to the Intercellular Bridge

To confirm that TEX14 localized to the intercellular bridge, the TEX14 expression pattern was compared to a known bridge component. Of the six known or suspected components of the intercellular bridge, five are broadly expressed cytoskeletal components that localize to multiple structures (Russel et al., 1987; Guttman et al, 1999; Tres et al., 1996; Johnson et al., 2004; and Kato et al., 2004). HSF2 is unique since it is the only non-cytoskeletal bridge component (Alastalo et al., 1994). Although HSF2 is broadly expressed, its strong localization to the intercellular bridge throughout meiosis contrasts clearly with its diffuse cytoplasmic and nuclear staining. Additionally, HSF2 localization to the intercellular bridge has been confirmed by immunoelectron microscopy (Alastalo et al., 1992). TEX14 colocalized with HSF2 (FIGS. 4B-4D) confirming the subcellular localization of TEX14 to the intercellular bridge.

### Example 15

### Bridge Structure

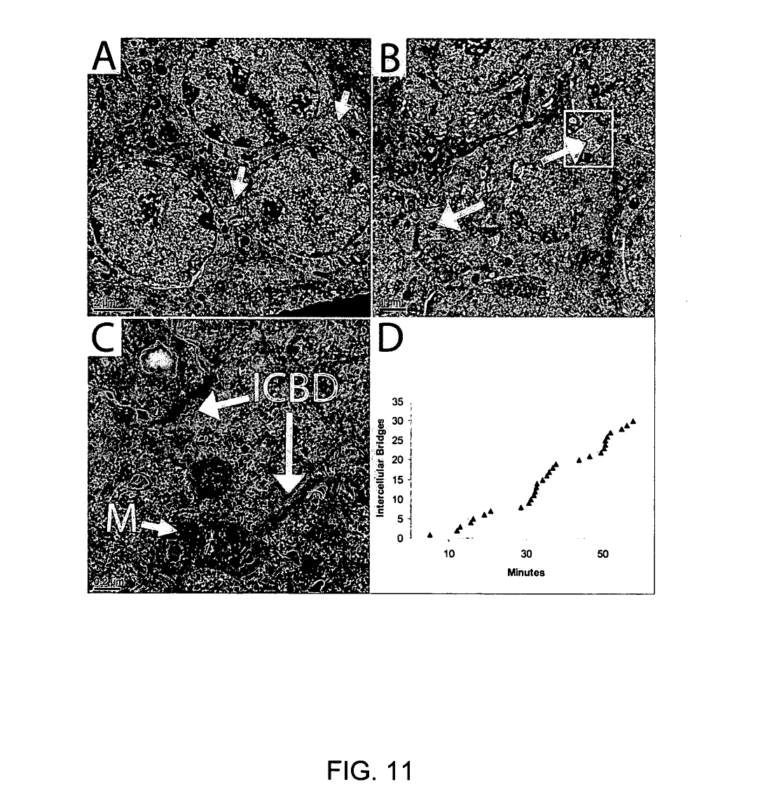

Since TEX14 localized to the intercellular bridges of germ cells, the bridge structure in the Tex14^{-/-} testes was examined by electron microscopy. Intercellular bridges between spermatocytes were readily located by their classic bridge densities in 11 day-old control testes (FIGS. 11A-11C) was, but were not detected in age matched Tex14^{-/-} null testes (FIG. 11D, indicating that TEX14 is essential for intercellular bridge structure in spermatocytes, in specific embodiments of the invention. To characterize this with an independent assay, the expression pattern of HSF2 in Tex14^{-/-} testes was examined. HSF2 localized to the intercellular bridge in control testes (FIG. 4C), while in Tex14^{-/-} testes only the nuclear and cytoplasmic components of HSF2 expression were seen (FIG. 4F) despite unaltered HSF2 protein expression (FIG. 4G).

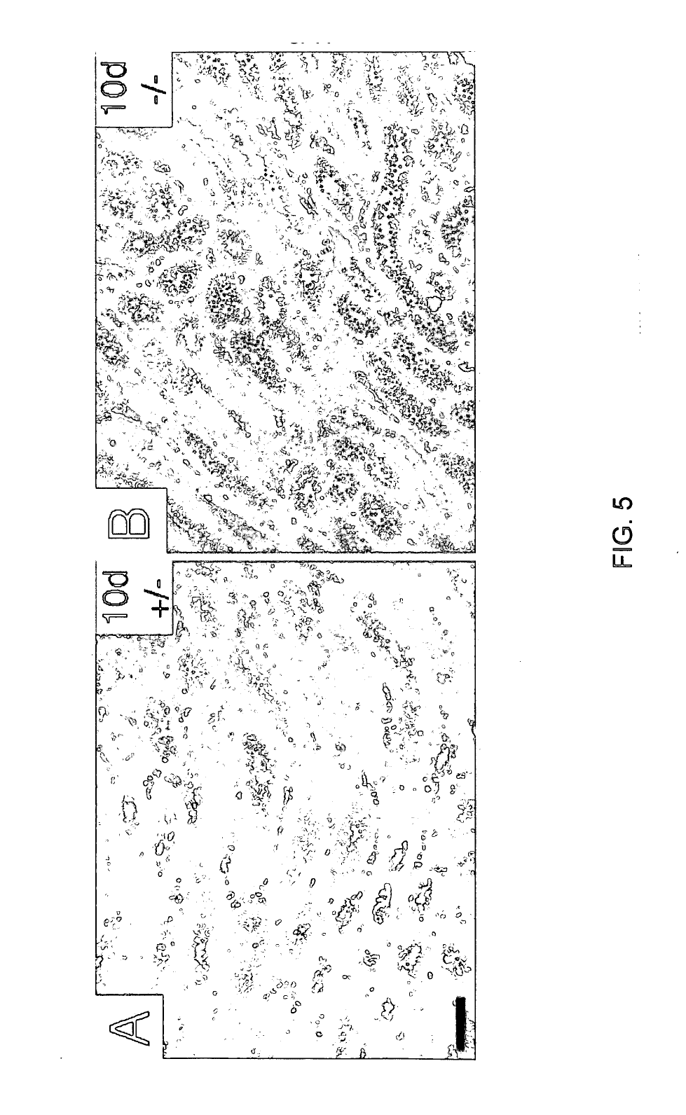

To further determine if TEX14 is required in spermatogonial intercellular bridges, we looked at the clonal organization of the spermatogonial syncytia formed by intercellular bridges. We used PLZF again as marker for spermatogonial stem cells and early dividing spermatogonia, (i.e., Aₛ, Aₚᵣ, and Aₐₗ) (Costoya et al., 2004; Buaas et al., 2004). It was found that TEX14 is in the intercellular bridges connecting PLZF-positive germ cells in neonatal and adult testes (FIGS. 5A, 5B). The organization of these early syncytia was then examined using whole mounted seminiferous tubules stained for PLZF. While 35 day-old control tubules contained single, paired, and aligned spermatogonia, 35 day-old Tex14^{-/-} tubules contained predominantly single spermatogonia with rare paired cells (possibly recently divided cells) and no aligned spermatogonia. It was concluded from the absence of PLZF-positive syncytium in Tex14^{-/-} tubules that TEX14 is required in early spermatogonial intercellular bridges.

Tex14^{-/-} mice demonstrate that spermatogenesis can progress without intercellular bridges through spermatogonial amplification and differentiation. However, spermatogenesis fails while the cells are still diploid but after the expression of early meiotic markers like SYCP3. Therefore, cytoplasmic contiguity is not essential for entry into meiosis. While significant apoptosis and loss of proliferation at meiosis is consistent with the “critical stage” hypothesis (Guo and Zheng, 2004; Robinson and Cooley, 1996; Stanley et al., 1972), a shared cytoplasmic factor that would influence germ cells progression through meiosis in not yet known. Apoptosis is also a normal part of spermatogenesis, and there is evidence that apoptotic signals can be transmitted throughout the syncytium (Huckins, 1978a; Huckins, 1978b; Hamer et al., 2003) or confined to one cell (Hamer et al., 2003) by intercellular bridges.

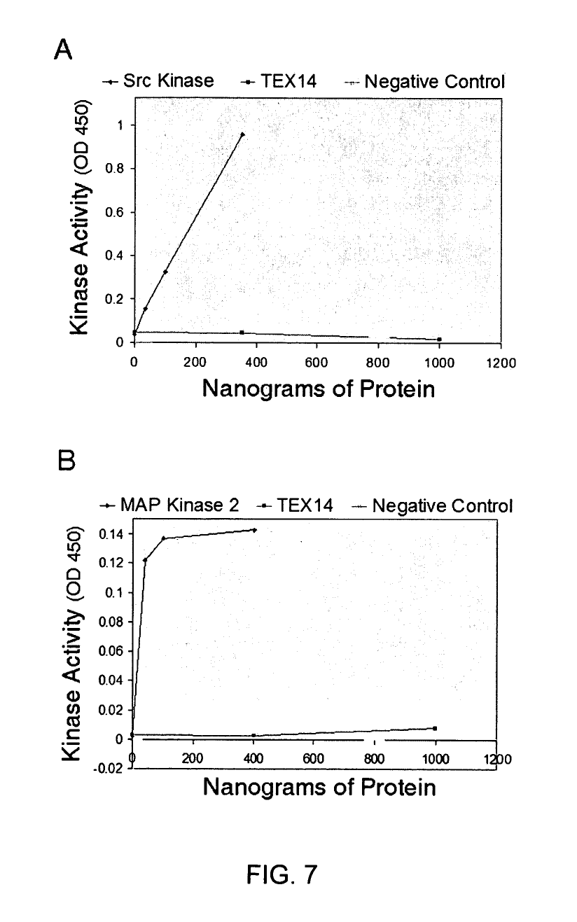

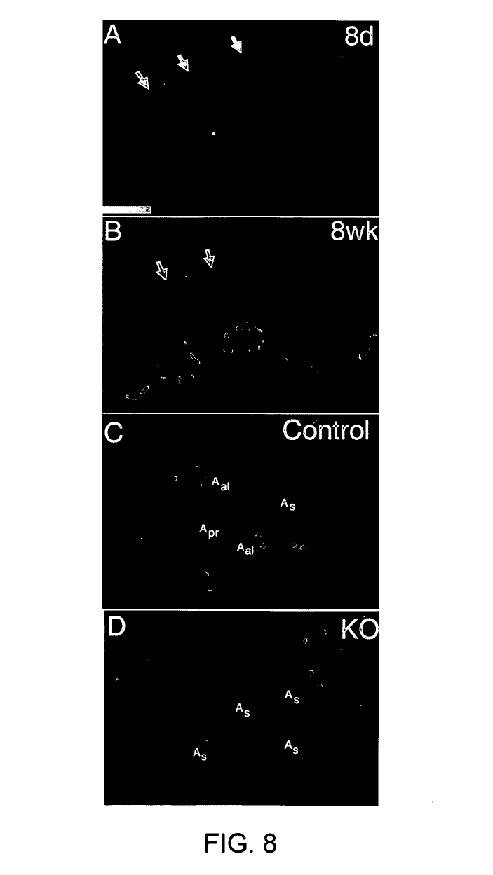

While our understanding of cytokinesis in somatic cells is rapidly advancing, little is known about the unique aspects of cytokinesis and intercellular bridge development in mammalian germ cells. In mammals, TEX14 is required for formation, maintenance, and/or stability of the intercellular bridge. In recruiting HSF2 and other bridge components, TEX14 most likely acts as a scaffold for intercellular bridge development rather than a kinase since there are unconventional amino acid replacements in the kinase-like domain of TEX14 (Wu et al., 2003; Hanks et al., 1988; Caenepeel et al., 2004), and TEX14 lacks kinase activity on generic substrates in standard tyrosine and serine/threonine kinase assays (FIGS. 7A, 7B). That said, the TEX14 kinase-like domain may play a novel, non-structural function in the intercellular bridge. In additional embodiments, other functions of TEX14 exist.

### Example 16

### Additional Bridge Components

Proteomic analysis on an enriched intercellular bridge fraction was employed by standard methods in the art. In particular embodiments, TEX14 interacts directly with at least some of the identified proteins. Table 2 provides a list of 126 exemplary candidates for direct interaction with TEX14 with their identifier numbers from the National Center for Biotechnology Informations' UniGene database. These have at least 5 unique peptide fragments identified by mass spectrometry in the proteomic analysis.

TABLE 2
Exemplary Potential TEX14 Binding Partners

| | | |
|---|---|---|
| Slice 1 (>188 kD) | | |
| Spectrin alpha chain | Mm.200611 | cytoskeleton |
| Spectrin beta 2 | Mm.123110 | cytoskeleton |
| Nonmuscle heavy chain myosin II-A | Mm.29677 | motor protein |
| Testis expressed gene 14 | Mm.103080 | High testis representation |
| | | in Unigene |
| Intercellular bridge protein 1 (ICB1); similar to | Mm.358894 | High testis representation |
| KIAA1210 protein | | in Unigene |
| Dynein heavy chain, cytosolic | Mm.181430 | motor protein |
| cytoskeleton associated protein 5 (Ckap5) (CH- | Mm.314907 | cytoskeleton |
| TOG) | | |
| AHNAK nucleoprotein isoform 1 | Mm.203866 | cytoskeleton/nuclear |
| Myh10 | Mm.218233 | motor protein |
| Plectin 10 | Mm.234912 | cytoskeleton |
| Filamin A (Alpha-filamin) | Mm.295533 | cytoskeleton |
| Myh11 protein | Mm.250705 | motor protein |
| DnaJ (Hsp40) homolog, subfamily C, member 13 | Mm.217256 | Heat shock protein/endocytosis |
| A-kinase anchor protein 9 | Mm.46044 | cytoskeleton/scaffold for |
| | | signalling |
| Pericentriolar material-1 | Mm.117896 | centrosome |
| Spectrin beta 3 | Mm.329668 | cytoskeleton |
| ataxia telangiectasia and rad3 related | Mm.212462 | kinase/meiosis/DNA repair |
| pericentrin 2 | Mm.251794 | centrosome |
| Kinesin family member 23 (MKLP1) | Mm.259374 | midbody |
| Microtubule-actin crosslinking factor 1 (ACF7) | Mm.3350 | cytoskeleton |
| Talin 1 | Mm.208601 | cytoskeleton/focal adhesions |
| centrosomal protein 2 | Mm.288729 | centrosome |
| Clathrin heavy polypeptide | Mm.254588 | vesicle trafficking |
| Type VI collagen alpha 3 subunit | Mm.7562 | extracellular matrix |
| Slice 2 (98-188 kD) | | |
| Ste20-like kinase (SLK) | Mm.281011 | kinase |
| Cyfip1 protein | Mm.37249 | ribosome (synaptic prep) |
| Intercellular bridge protein 2 (ICB2; which may | Mm.295940 | unknown fuction, High testis |
| also be referred to as GM817) | | representation in Unigene |
| DNA topoisomerase II, alpha isozyme | Mm.4237 | nuclear/dna |
| Band 4.1-like protein 2 (Generally expressed | Mm.306026 | cytoskeleton |
| protein 4.1) | | |
| Collagen alpha 1(VI) chain precursor | Mm.2509 | extracellular matrix |
| CDC42-binding protein kinase beta | Mm.27397 | signalling cascade |
| MutS homolog 6 | Mm.18210 | nuclear/dna repair |
| Slice 3 (62-98 kD) | | |
| Heat shock cognate 71 kDa protein | Mm.336743 | chaperone |
| 78 kDa glucose-regulated protein precursor | Mm.330160 | Chaperone; (not in bridge; Cell |
| (GRP 78) ((BiP) | | Tiss. Res. 2004; 316(3): 359-367) |
| Aconitate hydratase, mitochondrial precursor | Mm.154581 | mitochondria |
| (Citrate hydro-lyase) (Aconitase) | | |
| Endoplasmin precursor (94 kDa glucose- | Mm.87773 | chaperone |
| regulated protein) (GRP94) | | |
| heat shock protein 1 beta | Mm.2180 | chaperone |
| hydroxyacyl-Coenzyme A dehydrogenase/3- | Mm.200497 | mitochondria |
| ketoacyl-Coenzyme A thiolase/enoyl-Coenzyme | | |
| A hydratase (trifunctional protein), alpha subunit | | |
| Rac GTPase-activating protein (MgcRacGAP) | Mm.370666 | midbody |
| (Racgap1 protein) | | |
| Elongation factor 2 (EF-2) | Mm.289431 | ribosome |
| lamin B1 | Mm.4105 | nuclear |
| Heterogenous nuclear ribonucleoprotein U | Mm.2115 | nuclear |
| Carnitine O-palmitoyltransferase II, mitochondrial | Mm.307620 | mitochondria |
| precursor | | |
| Radixin | Mm.245746 | cytoskeletal |
| ezrin (villin 2) | Mm.277812 | cytoskeletal |
| Adapter-related protein complex 2 alpha 1 | Mm.253090 | vesicle trafficking/endocytosis |
| subunit (Alpha-adaptin A) | | |
| AP2B1 protein | Mm.39053 | vesicle trafficking/endocytosis |
| Moesin | Mm.138876 | cytoskeletal |
| Ribophorin I | Mm.188544 | ER/oligosaccharyltransferase |
| Annexin A6 (Annexin VI) | Mm.265347 | membrane and Ca²⁺ associated |
| | | (K/O is fertile, no phenotype) |
| poly (ADP-ribose) polymerase family, member 1 | Mm.277779 | DNA repair |
| Slice 4 (49-62 kD) | | |
| vimentin | Mm.268000 | cytoskeleton |
| centrosomal protein 55 | Mm.9916 | midbody and centrosome |
| ATP synthase alpha chain, mitochondrial | Mm.276137 | mitochondria |
| precursor | | |
| Beta-tubulin | Mm.216135 | mitochondria |
| M2-type pyruvate kinase | | |
| ATP synthase beta chain, mitochondrial | Mm.238973 | mitochondria |
| precursor | | |
| Tubulin alpha chain | Mm.370346 | cytoskeletal |
| EF-1 alpha | Mm.335315 | Ribosome |
| Glutamate dehydrogenase 1 | Mm.10600 | mitochondria |
| Hydroxymethylglutaryl-CoA synthase | Mm.289131 | mitochondria |
| P58(IPK), DnaJ (Hsp40) homolog, subfamily C, | Mm.12616 | chaperone |
| member 3 | | |
| malic enzyme | Mm.148155 | other |
| Grp58, protein disulfide isomerase associated 3 | Mm.263177 | chaperone, ER |
| HNRPK protein | Mm.378942 | RNA and DNA interacting |
| Src | Mm.22845 | kinase, signalling cascade |
| chaperonin containing T-complex polypeptide 1, | Mm.360232 | chaperone |
| subunit zeta | | |
| Annexin A11 (Annexin XI) | Mm.294083 | midbody, membrane and |
| | | calcium associated |
| EH-domain containing 1 | Mm.30169 | vesicle trafficking/endocytosis |
| chaperonin containing T-complex protein 1, delta | Mm.296985 | chaperone |
| subunit | | |
| chaperonin containing T-complex protein 1, eta | Mm.289900 | chaperone |
| subunit | | |
| aldehyde dehydrogenase 4 family, member A1 | Mm.273571 | mitochondria |
| Septin 7 | Mm270259 | |
| Slice 5 (38-49 kD) | | |
| Beta-Actin | Mm.297 | cytoskeletal |
| Glutamate oxaloacetate transaminase-2 | Mm.383179 | mitochondria |
| Annexin A2 (Annexin II) | Mm.238343 | midbody, membrane and |
| | | calcium associated |
| DnaJ homolog subfamily B, member 11 | Mm.37516 | chaperone |
| 3-hydroxy-3-methylglutaryl-CoA synthase 2 | Mm.289131 | mitochondria |
| Testis-specific protein TES101RP | Mm.23385 | testis specific |
| Elongation factor Tu, mitochondrial precursor | Mm.197829 | mitochondria |
| NADP+-specific isocitrate dehydrogenase | Mm.246432 | mitochondria |
| acetyl-coenzyme A acyltransferase 2 | Mm.245724 | mitochondria |
| Citrate synthase, mitochondrial precursor | Mm.58836 | mitochondria |
| acetyl-coenzyme A acetyltransferase 1 | Mm.293233 | mitochondria |
| Septin-2 | Mm.242324 | midbody |
| adaptor protein complex AP-2, mu1 | Mm.18946 | vesicle trafficking/endocytosis |
| Glyceraldehyde-3-phosphate dehydrogenase | Mm.379644 | other |
| Low density lipoprotein receptor-related protein | Mm.277661 | other |
| associated protein 1 | | |
| Hydroxyacyl-Coenzyme A dehydrogenase/3- | Mm.291463 | mitochondria |
| ketoacyl-Coenzyme A thiolase/enoyl-Coenzyme | | |
| A hydratase (trifunctional protein), beta subunit | | |
| Fumarate hydratase, mitochondrial precursor | Mm.41502 | mitochondria |
| Import inner membrane translocase subunit | Mm.195249 | mitochondria |
| TIM44, mitochondrial precursor | | |
| DnaJ homolog subfamily A member 2 | Mm.279692 | chaperone |
| Acyl-CoA dehydrogenase, medium-chain | Mm.10530 | mitochondria |
| specific, mitochondrial precursor | | |
| Slice 6 (30-38 kD) | | |
| Dodecenoyl-Coenzyme A delta isomerase | Mm.291743 | mitochondria |
| Solute carrier family 25, member 31 | Mm.78691 | testis specific, mitochondria |
| Sideroflexin 3 | Mm.36169 | mitochondria |
| Malate dehydrogenase 2 | Mm.297096 | mitochondria |
| histone 1, H1c | Mm.193539 | nuclear |
| Echs1 protein | Mm.24452 | mitochondria |
| Thiosulfate sulfurtransferase | Mm.15312 | mitochondria |
| Short chain 3-hydroxyacyl-CoA dehydrogenase, | Mm.260164 | mitochondria |
| mitochondrial precursor | | |
| Vesicle-associated membrane protein- | Mm.266767 | vesicle trafficking |
| associated protein A (VAMP- associated protein | | |
| A) | | |
| Annexin A4 (Annexin IV) | Mm.259702 | membrane and Ca²⁺ associated |
| succinate-CoA ligase, GDP-forming, alpha | Mm.29845 | mitochondria |
| subunit | | |
| Cytochrome c-1 | Mm.29196 | mitochondria |
| Prohibitin | Mm.263862 | mitochondria |
| Guanine nucleotide-binding protein beta subunit | Mm.5305 | signalling cascade |
| 2-like 1 (RACK1) | | |
| quinoid dihydropteridine reductase | Q8BVI4 | mitochondria |
| VDAC-1 | Mm.3555 | mitochondria |
| hydroxyacyl-Coenzyme A dehydrogenase type II | Mm.6994 | mitochondria |
| pyruvate dehydrogenase (lipoamide) beta | Mm.301527 | mitochondria |
| isocitrate dehydrogenase 3 (NAD+) alpha | Mm.279195 | mitochondria |
| 3-hydroxy-3-methylglutaryl-Coenzyme A lyase | Mm.22668 | mitochondria |
| Slice 7 (20-30 kD) | | |
| Peroxiredoxin 1 (Thioredoxin peroxidase 2) | Mm.30929 | metabolic |
| peptidylprolyl isomerase b | Mm.325816 | other |
| ATP synthase, H+ transporting, mitochondrial F1 | Mm.297484 | mitochondrial |
| complex, O subunit (Atp5o) | | |
| Manganese superoxide dismutase | Mm.290876 | mitochondrial |
| SEC22b | Mm.2551 | ER/Golgi vesicle trafficking |
| 1700029F09Rik | Mm.348017 | other |
| Glutathione S-transferase Mu 1 | Mm.378928 | other |
| ubiquinol-cytochrome c reductase iron-sulfer | Mm.181933 | mitochondrial |
| subunit (Uqcrfs1) | | |
| Slice 8 (15-20 kD) | | |
| histone H4 | Mm.228709 | nuclear |
| Cytochrome c, somatic | Mm.378903 | mitochondrial |
| Similar to arginine-rich, mutated in early stage | Mm.29778 | other |
| tumors | | |
| RhoG | Mm.259795 | signalling cascade |
| Slice 9 (8-15 kD) | | |
| hemoglobin, beta adult major chain (Hbb-b1) | Mm.288567 | contaminant |
| | | Metabolic |
| sterol carrier protein x | Mm.379011 | |
| Slice 10 (5-8 kD) | | |
| None | | |

| category | # Candidates | % of total |
|---|---|---|
| mitochondria | 41 | 32.3 |
| cytoskeleton | 17 | 13.4 |
| other | 16 | 12.6 |
| chaperone | 11 | 8.7 |
| nuclear | 10 | 7.9 |
| vesicle trafficking | 7 | 5.5 |
| signalling cascade | 6 | 4.7 |
| centrosome | 5 | 3.9 |
| motor protein | 5 | 3.9 |
| Testis by Unigene | 4 | 3.1 |
| ribosome | 3 | 2.4 |
| ER | 2 | 1.6 |

Cytokinesis proteins with under 5 peptides (9 total)

| | |
|---|---|
| Slice1 | |
| NUMA1 | Mm.27259 |
| Slice2 | |
| IQGAP1 | Mm.207619 |
| Eg5/KIF11 | Mm.42203 |
| Slice4 | |
| Septin 9 | Mm.38450 |
| Slice5 | |
| Erk2 | Mm.196581 |
| Arp3 | Mm.183102 |
| Slice6 | |
| Arp2 | Mm.379122 |
| Slice7 | |
| Arp2/3 complex 21 kD subunit | Mm.275942 |
| Slice8 | |
| Myosin light chain | Mm.347786 |

In particular embodiments of the invention, TEX14 interacts with intercellular bridge protein 1 (ICB1, which may also be referred to as “similar to KIAA1210”) and/or intercellular bridge protein 2 (ICB2, which may also be referred to as “GM817”). An exemplary human ICB1 sequence is provided in SEQ ID NO:13; an exemplary mouse ICB1 polypeptide sequence is provided in SEQ ID NO:14; and an exemplary mouse ICB1 cDNA sequence is provided in SEQ ID NO:15. Also, an exemplary mouse ICB2 DNA sequence is provided in SEQ ID NO:16; an exemplary mouse ICB2 polypeptide sequence is provided in SEQ ID NO:17; an exemplary human ICB2 mRNA sequence is provided in SEQ ID NO:18; and an exemplary human ICB2 polypeptide sequence is provided in SEQ ID NO:19.

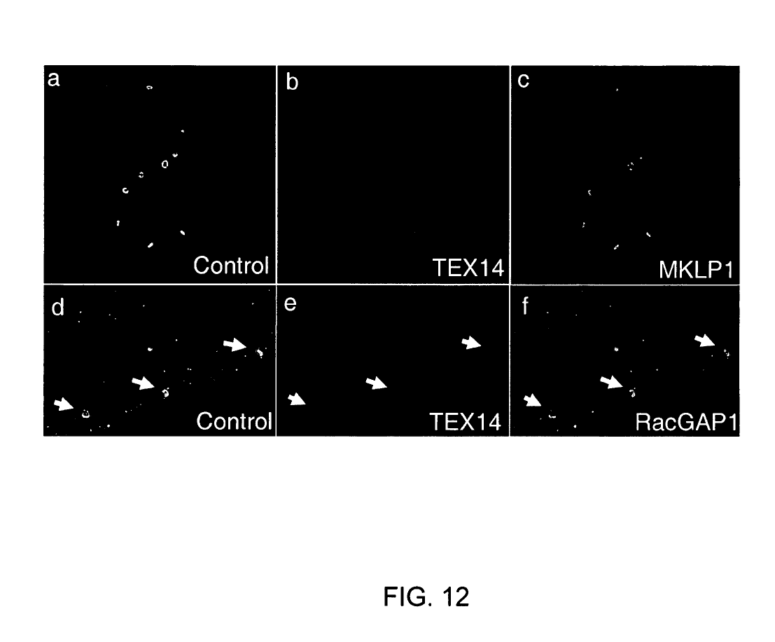

FIG. 12 illustrates that MKLP1 and RacGAP1, which form the centralspindlin complex, colocalize with TEX14 in the intercellular bridge and are considered components of the mammalian intercellular bridge. MKLP1 (FIG. 12C) and TEX14 (FIG. 12B) colocalize in the intercellular bridges of 14 day-old mouse testis (FIG. 12A). RacGAP1 (FIG. 12F) and TEX14 (FIG. 12E) also colocalize in 14 day-old mouse intercellular bridges (FIG. 12D). Arrows in FIGS. 12D-12F indicate intercellular bridges.

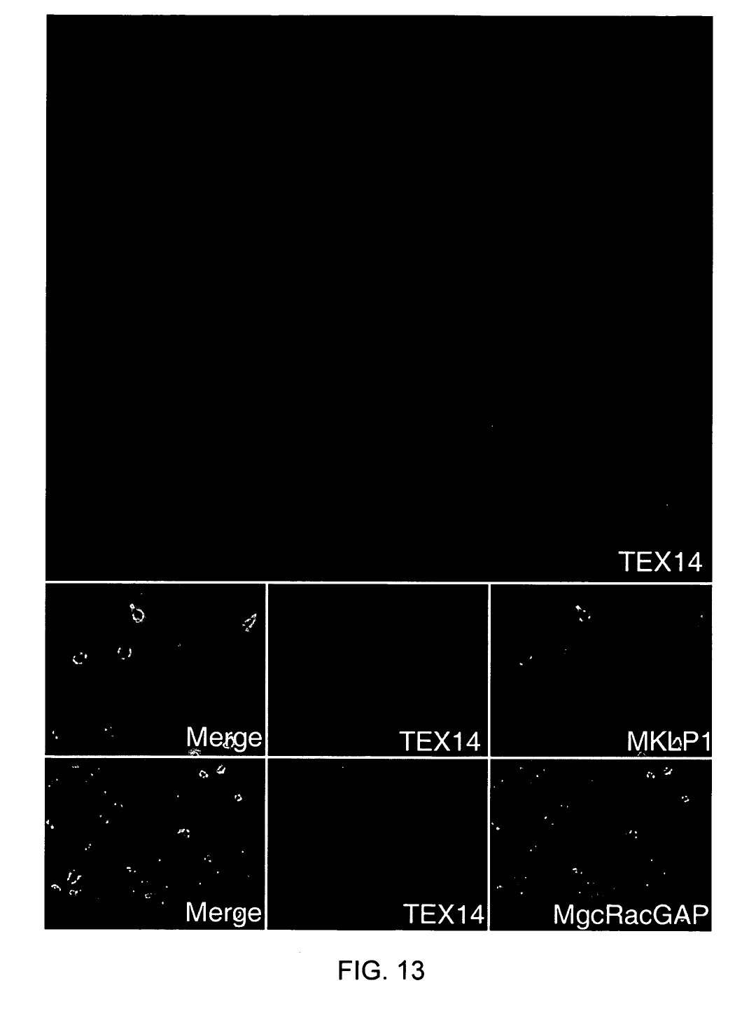

FIG. 13 demonstrates human tissue stained for TEX14, which clearly shows that TEX14 is also in human intercellular bridges. The lower panels demonstrate that MKLP1 and MgcRacGAP colocalize with TEX14 in human intercellular bridges.

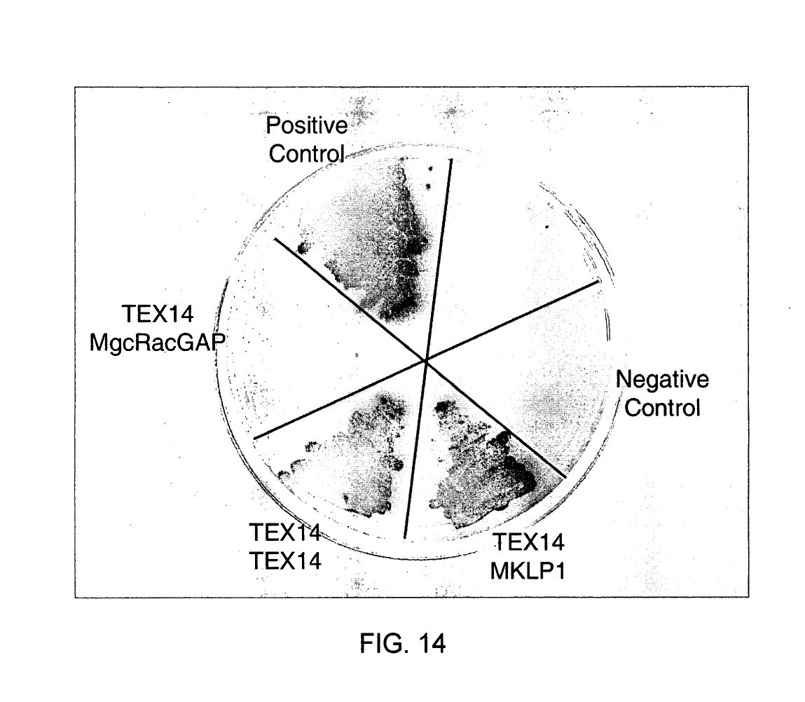

FIG. 14 illustrates exemplary targeted yeast-two-hybrid interactions between TEX14 and exemplary centralspindlin proteins. In this particular type of assay, TEX14 interacts strongly with itself and MKLP1 but very weakly with MgcRacGAP by targeted yeast-two-hybrid analysis. In other embodiments, interaction between TEX14 and MgcRacGAP is not weak, as detected by other assays. Murine p53 interacts with and SV40 large T-antigen in the positive control, and human Lamin C fails SV40 large T-antigen in the negative control.

### Example 17

### Significance of the Present Invention

This work identifies Tex14 as the first gene essential for mammalian intercellular bridge development. In invertebrates, the intercellular bridge is a dynamic structure that develops with the germ cells. In Drosophila, ovarian ring canals (intercellular bridges) sequentially incorporate essential components as they mature. Mutations in genes for these essential ring canal components cause sterility and have shown that the primary role of the ring canal in Drosophila is to accommodate cytoplasmic transfer from nurse cells to the oocyte. The intercellular bridges in mammals also appear to go through a developmental process. HSF2 is not incorporated into the intercellular bridge until meiosis, and Protocadherin alpha3 is only present in haploid cells.

The role of HSF2, a transcription factor, at the intercellular bridge is unknown; however, male hsf2^{-/-} mice have significantly decreased testis size and sperm number, but only a small decrease in fertility (Wang et al., 2003). Although, hsf2^{-/-} mice display a partial disruption of spermatogenesis during meiosis, the hsf2^{-/-} phenotype can not be explained by absence of the intercellular bridge since TEX14 labels the bridges of male hsf2^{-/-} mice. In recruiting HSF2, TEX14 most likely acts as a scaffold for intercellular bridge development rather than a kinase since there are unconventional amino acid replacements in the kinase-like domain of TEX14 and TEX14 lacks kinase activity on generic substrates in standard tyrosine and serine/threonine kinase assays (FIGS. 7A and 7B). That said, the TEX14 kinase-like domain may play a novel, non-structural function in the intercellular bridge. Since spermatogenic disruption in the Tex14^{-/-} testes was first detected at the meiotic phase, despite the presence of intercellular bridges among the earlier mitotic germ cells (i.e., spermatogonia), the addition of TEX14 to the intercellular bridge prior to meiosis was essential for the functional transition from a mitotic to meiotic intercellular bridge. This dynamic character of the mammalian intercellular bridge was necessary for completion of meiosis. Lastly, the persistence of stem cells and spermatogonia and normal testosterone levels in sterile Tex14^{-/-} adults indicated that TEX14 and its interacting proteins were novel targets for male contraception.

### Example 18

### Screening for Targets

Firstly, the below protocol describes the production of an enriched isolation of TEX14-comprising intercellular bridges. The protocol has several features that make it attractive for screening compounds that can displace components of the intercellular bridge, and it can be adapted to screen for labeled compounds that bind to the intercellular bridge. An overview is given below. The highlights are as follows:

1) Rapid—One day protocol. 3 hours for isolation plus the time necessary to incubate with candidate compounds and detect the results.

2) Scalable—protocol can be adapted for high throughput 96 well format

3) Nondisruptive to the bridge—TEX14, HSF2, and delta tubulin remain in the bridge after enrichment.

4) Inexpensive—standard centrifuges and inexpensive reagents used

5) Two enrichment steps—The first enrichment step is isotonic and uses no detergent leaving the bridges in an in vivo state for compound interactions. The second step removes membrane and many non-bridge components leaving an enriched bridge fraction that can be detected in vitro.

6) Adaptable—any component of the intercellular bridge for which an antibody or specific ligand exists can be screened for displacement from the bridge

7) Internal controls—Some cytoskeletal components are also enriched with the intercellular bridge. Measuring their levels (e.g., fluorescently tagged phalloidin to measure f-actin) serves as an internal loading control, plus compounds that disrupt the bridge through non-specific cytoskeletal interaction are identified.

The strategy for isolating intercellular bridges is based on the principle developed during the late 1960's and 1970's for isolation of neuronal synaptosomes and postsynaptic densities. While known in the neuroscience field, these principles are not known in the reproductive field, and it was not initially obvious that they could be applied to the intercellular bridge.

More specifically the protocol entails the following steps:

1) Isolate testis

2) Remove seminiferous tubules from tunica in isolation buffer

3) Finely mince tissue with scissors and take up in 900 ul of isolation buffer per adult testicle (˜10% w/v)

4) Homogenize tissue 10 strokes in Teflon/glass dounce homogenizer plus optional 10 passes through a 26 gauge syringe

5) Spin at 720 g 15 minutes

6) Spin supernatant at 3,500 g 15 minutes

7) Resuspend pellet in lysis buffer

8) Incubate at 4 degrees on shaker for 20 minutes

9) Spin down enriched bridge fraction at 3,500 g for 15 min

Isolation buffer: (0.32M sucrose, 4 mM HEPES, pH 7.4)

Lysis buffer: (0.5% Triton X-100, 4 mM HEPES, pH 7.4)

In vivo bridges may be treated with a compound between steps 6 and 7 if the compound is membrane permeable. Bridges may also be treated after step 9 if the compound is membrane impermeable. Detection is performed by quantifying the amount of target protein left in the enriched bridge fraction after treatment with a compound. This is done with a labeled antibody for the target or a labeled specific ligand.

### REFERENCES

All patents and publications mentioned in the specifications are indicative of the levels of those skilled in the art to which the invention pertains. All patents and publications are herein incorporated by reference to the same extent as if each individual publication was specifically and individually indicated to be incorporated by reference.
Alastalo, T. P. et al. Stage-specific expression and cellular localization of the heat shock factor 2 isoforms in the rat seminiferous epithelium. Exp Cell Res 240, 16-27 (1998).
Bettocchi, C., Parkinson, M. C., Ralph, D. J. & Pryor, J. P. Clinical aspects associated with Sertoli-cell-only histology. Br J Urol 82, 534-7 (1998).
Bradley, A. Production and analysis of chimeric mice-teratocarcinomas and embryonic stem cells: a practical approach. in Production and analysis of chimeric mice (ed. Robinson, E. J.) 113-151 (Oxford: IRL, London, 1987).
Brill, J. A., Hime, G. R., Scharer-Schuksz, M. & Fuller, M. T. A phospholipid kinase regulates actin organization and intercellular bridge formation during germline cytokinesis. Development 127, 3855-64 (2000).
Caenepeel, S., Charydczak, G., Sudarsanam, S., Hunter, T. & Manning, G. The mouse kinome: discovery and comparative genomics of all mouse protein kinases. Proc Natl Acad Sci USA 101, 11707-12 (2004).
Dobson, M. J., Pearlman, R. E., Karaiskakis, A., Spyropoulos, B. & Moens, P. B. Synaptonemal complex proteins: occurrence, epitope mapping and chromosome disjunction. J Cell Sci 107 ( Pt 10), 2749-60 (1994).
Dong, J. et al. Growth differentiation factor-9 is required during early ovarian folliculogenesis. Nature 383, 531-535 (1996).
Dym, M. & Fawcett, D. W. Further observations on the numbers of spermatogonia, spermatocytes, and spermatids connected by intercellular bridges in the mammalian testis. Biol Reprod 4, 195-215 (1971).
Guttman, J. A., Mulholland, D. J. & Vogl, A. W. Plectin is concentrated at intercellular junctions and at the nuclear surface in morphologically differentiated rat Sertoli cells. Anat Rec 254, 418-28 (1999).
Hanks, S. K., Quinn, A. M. & Hunter, T. The protein kinase family: conserved features and deduced phylogeny of the catalytic domains. Science 241, 42-52 (1988).
Hime, G. R., Brill, J. A. & Fuller, M. T. Assembly of ring canals in the male germ line from structural components of the contractile ring. J Cell Sci 109 ( Pt 12), 2779-88 (1996).
Hudson, A. M. & Cooley, L. A subset of dynamic actin rearrangements in Drosophila requires the Arp2/3 complex. J Cell Biol 156, 677-87 (2002).
Johnson, K. J., Zecevic, A. & Kwon, E. J. Protocadherin alpha3 acts at sites distinct from classic cadherins in rat testis and sperm. Biol Reprod 70, 303-12 (2004).
Kato, A., Nagata, Y. & Todokoro, K. Delta-tubulin is a component of intercellular bridges and both the early and mature perinuclear rings during spermatogenesis. Dev Biol 269, 196-205 (2004).
Kumar, T. R., Wang, Y., Lu, N. & Matzuk, M. M. Follicle stimulating hormone is required for ovarian follicle maturation but not male fertility. Nature Genetics 15, 201-204 (1997).
Lu, N., Guarnieri, D. J. & Simon, M. A. Localization of Tec29 to ring canals is mediated by Src64 and PtdIns(3,4,5)P(3)-dependent mechanisms. Embo J 23, 1089-100 (2004).
Matzuk, M. M., Finegold, M. J., Su, J.-G. J., Hsueh, A. J. W. & Bradley, A. a-Inhibin is a tumor-suppressor gene with gonadal specificity in mice. Nature 360, 313-319 (1992).
Moens, P. B. et al. The time course and chromosomal localization of recombination-related proteins at meiosis in the mouse are compatible with models that can resolve the early DNA-DNA interactions without reciprocal recombination. J Cell Sci 115, 1611-22 (2002).
Rajkovic, A., Yan, C., Klysik, M. & Matzuk, M. M. Discovery of germ cell-specific transcripts by expressed sequence tag database analysis. Fertil Steril 76, 550-4 (2001).
Ravnik, S. E. & Wolgemuth, D. J. Regulation of meiosis during mammalian spermatogenesis: the A-type cyclins and their associated cyclin-dependent kinases are differentially expressed in the germ-cell lineage. Dev Biol 207, 408-18 (1999).
Robinson, D. N., Cant, K. & Cooley, L. Morphogenesis of Drosophila ovarian ring canals. Development 120, 2015-25 (1994).
Rossi, P., Sette, C., Dolci, S. & Geremia, R. Role of c-kit in mammalian spermatogenesis. J Endocrinol Invest 23, 609-15 (2000).
Roy, A., Yan, W., Burns, K. H. & Matzuk, M. M. Tektin3 encodes an evolutionarily conserved putative testicular microtubule-related protein expressed preferentially in male germ cells. Mol Reprod Dev, In press (2003).
Russell, L. D., Vogl, A. W. & Weber, J. E. Actin localization in male germ cell intercellular bridges in the rat and ground squirrel and disruption of bridges by cytochalasin D. Am J Anat 180, 25-40 (1987).
Sokol, N. S. & Cooley, L. Drosophila filamin encoded by the cheerio locus is a component of ovarian ring canals. Curr Biol 9, 1221-30 (1999).
Sweeney, C. et al. A distinct cyclin A is expressed in germ cells in the mouse. Development 122, 53-64 (1996).
Tres, L. L., Rivkin, E. & Kierszenbaum, A. L. Sak 57, an intermediate filament keratin present in intercellular bridges of rat primary spermatocytes. Mol Reprod Dev 45, 93-105 (1996).
Ventela, S., Toppari, J. & Parvinen, M. Intercellular organelle traffic through cytoplasmic bridges in early spermatids of the rat: mechanisms of haploid gene product sharing. Mol Biol Cell 14, 2768-80 (2003).
Wang, G., Zhang, J., Moskophidis, D. & Mivechi, N. F. Targeted disruption of the heat shock transcription factor (hsf)-2 gene results in increased embryonic lethality, neuronal defects, and reduced spermatogenesis. Genesis 36, 48-61 (2003).
Weber, J. E. & Russell, L. D. A study of intercellular bridges during spermatogenesis in the rat. Am J Anat 180, 1-24 (1987).
Wu, M. H. et al. Sequence and expression of testis-expressed gene 14 (Tex14): a gene encoding a protein kinase preferentially expressed during spermatogenesis. Gene Expr Patterns 3, 231-236 (2003).
Yan, W., Kero, J., Huhtaniemi, I. & Toppari, J. Stem cell factor functions as a survival factor for mature Leydig cells and a growth factor for precursor Leydig cells after ethylene dimethane sulfonate treatment: implication of a role of the stem cell factor/c-Kit system in Leydig cell development. Dev Biol 227, 169-82 (2000).
Yan, W., Ma, L., Burns, K. H. & Matzuk, M. M. Haploinsufficiency of kelch-like protein homolog 10 causes infertility in male mice. Proc Natl Acad Sci USA 101, 7793-8 (2004).
Yan, W., Ma, L., Burns, K. H. & Matzuk, M. M. HILS1 is a spermatid-specific linker histone H1-like protein implicated in chromatin remodeling during mammalian spermiogenesis. Proc Natl Acad Sci USA 100, 10546-51 (2003).
Yan, W., Suominen, J. & Toppari, J. Stem cell factor protects germ cells from apoptosis in vitro. J Cell Sci 113 ( Pt 1), 161-8 (2000).

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A composition comprising a compound that disrupts intercellular bridges that interconnect germ cells, said composition admixed with a pharmaceutical carrier.

2. The composition of claim 1, wherein the compound decreases the expression and/or activity of TEX14.

3. The composition of claim 2, wherein the composition disrupts association of TEX14 with a binding partner.

4. The composition of claim 3, wherein the binding partner is TEX14, TIP1, TIP2, TIP3, TIP4, HSF2, actin, delta tubulin, plectin, spermatogenic cell/sperm-associated keratin of molecular mass 57 kDa (SAK 57), protocadherin alpha 3, intercellular bridge protein 1 (ICB1), intercellular bridge protein 2 (ICB2), MgcRacGap1 (male germ cell RacGap1), mitotic kinesin-like protein 1 (MKLP1/CHO1/Kif23), or a combination thereof.

5. The composition of claim 1, wherein the composition is further defined as a male contraceptive.

6. A method of modulating fertility in a male subject, comprising the step of administering to the subject the compound defined in claim 1 such that the composition disrupts the intercellular bridges.

7. The method of claim 6, wherein the disruption of the intercellular bridges results in a decrease in spermatogenesis.

8. The method of claim 6, wherein the disruption of the intercellular bridges results in apoptosis of spermatocytes, spermatogonia, or both.

9. The method of claim 6, wherein the disruption of the intercellular bridges results in altered spermatogensis.

10. The method of claim 6, wherein the disruption of the intercellular bridges results in apoptosis of germ cells.

11. A method for identifying a substance that disrupts germ cell intercellular bridge formation, maintenance, and/or function comprising the steps of:
administering the test substance to a mammalian subject or enriched intercellular bridge fraction; and
determining the effects of the substance on germ cell intercellular bridge function, such that the substance reduces the production or function of sperm in said subject when compared to a control substance.

12. A method of making a TEX14 inhibitor, comprising:
(a) providing a candidate substance suspected of decreasing TEX14 expression and/or activity;
(b) selecting the TEX14 inhibitor by assessing the ability of the candidate substance to decrease TEX14 expression; and
(c) making the selected TEX14 inhibitor.

13. The method of claim 12, wherein the candidate substance alters germ cell intercellular bridge function and/or maintenance.

14. The method of claim 12, wherein the candidate substance comprises a protein, a nucleic acid molecule, an organo-pharmaceutical, or a combination thereof.

15. The method of claim 12, wherein the providing step is further defined as providing in a cell or a cell-free system a TEX14 polypeptide and the TEX14 polypeptide is contacted with the candidate substance.

16. The method of claim 13, wherein the candidate substance is a protein.

17. A method of altering intercellular bridge formation, function and/or maintenance in a subject by administereing the inhibitor identified by the method of claim 12.

18. A method of decreasing fertility in a subject by administering the inhibitor identified by the method of claim 12.

19. A method of making a compound that inhibits germ cell intercellular bridge function and/or maintenance comprising:
(a) providing a candidate substance suspected of inhibiting the intercellular bridge function and/or maintenance;
(b) selecting the compound by assessing the ability of the candidate substance to inhibit the intercellular bridge function and/or maintenance; and
(c) making the selected compound.

20. The method of claim 17, wherein the compound is a TEX14 inhibitor.

21. A method of diganosing fertility in a subject by measuring the activity and/or expression of TEX14.

22. A kit comprising the composition of claim 1, wherein the composition is housed in a suitable container.

23. A kit comprising the inhibitor identified by the method of claim 12, wherein the inhibitor is housed in a suitable container.

24. A method of identifying an inhibitor of TEX14, comprising the steps of:
providing part or all of a TEX14 polypeptide, wherein said part or all of the TEX14 polypeptide comprises at least one TEX14 binding partner domain;
providing a TEX14 binding partner;
providing a test compound; and
assaying for absence of binding of TEX14 to its binding partner in the presence of the test compound.

25. The method of claim 24, further comprising the step of manufacturing the TEX14 inhibitor.

26. The method of claim 24, wherein the TEX 14 binding partner is TEX14, MKLP1, or MgcRacGAP.

27. The method of claim 24, further defined as the assaying step comprising yeast two hybrid.

28. The method of claim 24, wherein the inhibitor is further defined as a compound that inhibits germ cell intercellular bridge function and/or maintenance.

29. The method of claim 24, further comprising delivering the inhibitor to an individual.

30. The method of claim 29, wherein the individual is in need of treatment for infertility.

31. The method of claim 29, wherein the individual is in need of contraception.
